SEQUENCE LISTING (57 sequences)

SEQ ID NO: 1 - RNA, 3995 nt, synthetic construct
  source 1..3995: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 2 - RNA, 57 nt, synthetic construct
  source 1..57: mol_type = other RNA
gggaaataagagagaaaagaagagtaagaagaaatataagaccccggcgccgccacc

SEQ ID NO: 3 - RNA, 3819 nt, synthetic construct
  source 1..3819: mol_type = other RNA
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 4 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - RNA, 3986 nt, synthetic construct
  source 1..3986: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggc

SEQ ID NO: 7 - RNA, 3810 nt, synthetic construct
  source 1..3810: mol_type = other RNA
atgttcgtgttcctggtgctgctgcccctggtgagcagccagtgcgtgaacctgaccacccggacccagctgccaccagcctacaccaacagcttcacccggggcgtgtactaccccgacaaggtgttccggagcagcgtgctgcacagcacccaggacctgttcctgcccttcttcagcaacgtgacctggttccacgtgatcagcggcaccaacggcaccaagcggttcgacaaccccgtgctgcccttcaacgacggcgtgtacttcgccagcatcgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcatcgtgcgggagcccgaggacctgccccagggcttcagcgccctggagcccctggtggacctgcccatcggcatcaacatcacccggttccagaccctgctggccctgcaccggagctacctgaccccaggcgacagcagcagcggctggaccgccggcgccgccgcctactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacctggcccctttcttcaccttcaagtgctacggcgtgagccccaccaagctgaacgacctgtgcttcaccaacgtgtacgccgacagcttcgtgatccggggcgacgaggtgcggcagatcgccccaggccagaccggcaacatcgccgactacaactacaagctgcccgacgacttcaccggctgcgtgatcgcctggaacagcaacaagctggacagcaaggtgagcggcaactacaactacctgtaccggctgttccggaagagcaacctgaagcccttcgagcgggacatcagcaccgagatctaccaggccggcaacaagccctgcaacggcgtggccggcttcaactgctacttccctctgcggagctacagcttccggcccacctacggcgtgggccaccagccctaccgggtggtggtgctgagcttcgagctgctgcacgccccagccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggccgggacatcgccgacaccaccgacgccgtgcgggatccccagaccctggagatcctggacatcaccccttgcagcttcggcggcgtgagcgtgatcaccccaggcaccaacaccagcaaccaggtggccgtgctgtaccagggcgtgaactgcaccgaggtgcccgtggccatccacgccgaccagctgaccccaacctggcgggtgtacagcaccggcagcaacgtgttccagacccgggccggctgcctgatcggcgccgagtacgtgaacaacagctacgagtgcgacatccccatcggcgccggcatctgcgccagctaccagacccagaccaagagccaccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaagggcctgaccgtgctgcctcctctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagccggctggaccctcccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagctttccccagagcgcaccccacggcgtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc

SEQ ID NO: 8 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - RNA, 3983 nt, synthetic construct
  source 1..3983: mol_type = other RNA
gggaaataagagagaaaagaagagtaagaagaaatataagaccccggcgccgccaccatgttcgtgttcctggtgctgctgcccctggtgagcagccagtgcgtgaacctgatcacccggacccagagctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctgggccgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacttcgccccattcttcgccttcaagtgctacggcgtgagccccaccaagctgaacgacctgtgcttcaccaacgtgtacgccgacagcttcgtgatccgtggcaacgaggtgagccagatcgcacccggccagacaggcaacatcgccgactacaactacaagctgcccgacgacttcaccggctgcgtgatcgcctggaacagcaacaagctcgacagcaaggtgggcggcaactacaactaccggtaccggctgttccggaagagcaacctgaagcccttcgagcgggacatcagcaccgagatctaccaagccggcaacaagccttgcaacggcgtggccggcgtgaactgctacttccctctgcagagctacggcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggtgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaagtcacaccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcaggagagatagtggagtggtctttgaataaagtctgagtgggcggc

SEQ ID NO: 10 - RNA, 3804 nt, synthetic construct
  source 1..3804: mol_type = other RNA
atgttcgtgttcctggtgctgctgcccctggtgagcagccagtgcgtgaacctgatcacccggacccagagctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctgggccgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcgacgaggtgttcaacgccacccggttcgccagcgtgtacgcctggaaccggaagcggatcagcaactgcgtggccgactacagcgtgctgtacaacttcgccccattcttcgccttcaagtgctacggcgtgagccccaccaagctgaacgacctgtgcttcaccaacgtgtacgccgacagcttcgtgatccgtggcaacgaggtgagccagatcgcacccggccagacaggcaacatcgccgactacaactacaagctgcccgacgacttcaccggctgcgtgatcgcctggaacagcaacaagctcgacagcaaggtgggcggcaactacaactaccggtaccggctgttccggaagagcaacctgaagcccttcgagcgggacatcagcaccgagatctaccaagccggcaacaagccttgcaacggcgtggccggcgtgaactgctacttccctctgcagagctacggcttccggcccacctacggcgtgggccaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccagggtgtgaactgcaccgaggtgcccgtggccatccacgccgaccagctgacacccacctggcgggtctacagcaccggcagcaacgtgttccagacccgggccggttgcctgatcggcgccgagtacgtgaacaacagctacgagtgcgacatccccatcggcgccggcatctgtgccagctaccagacccagaccaagtcacaccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaagcgggccctgaccggcatcgccgtggagcaggacaagaacacccaggaggtgttcgcccaggtgaagcagatctacaagacccctcccatcaagtacttcggcggcttcaacttcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgccctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccacaacgcccaggccctgaacaccctggtgaagcagctgagcagcaagttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacc

SEQ ID NO: 11 - RNA, 122 nt, synthetic construct
  source 1..122: mol_type = other RNA
tgataataggctggagcctcggtggcctagcttcttgccccttgggcctccccccagcccctcctccccttcctgcaggagagatagtggagtggtctttgaataaagtctgagtgggcggc

SEQ ID NO: 12 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - RNA, 47 nt, synthetic construct
  source 1..47: mol_type = other RNA
gggaaataagagagaaaagaagagtaagaagaaatataagagccacc

SEQ ID NO: 14 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 15 - protein, 13 aa, synthetic construct
TQTNSPRRARSVA

SEQ ID NO: 16 - protein, 8 aa, synthetic construct
KPSKRSFI

SEQ ID NO: 17 - RNA, 11 nt, synthetic construct
  source 1..11: mol_type = other RNA
gggatcctacc

SEQ ID NO: 18 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 19 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 20 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 21 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 22 - RNA, 59 nt, synthetic construct
  source 1..59: mol_type = other RNA
ggagaacttccgcttccgttggcgcaagcgctttcattttttctgctaccgtgactaag

SEQ ID NO: 23 - RNA, 46 nt, synthetic construct
  source 1..46: mol_type = other RNA
ggaaataagagagaaaagaagagtaagaagaaatataagagccacc

SEQ ID NO: 24 - RNA, 56 nt, synthetic construct
  source 1..56: mol_type = other RNA
ggaaataagagagaaaagaagagtaagaagaaatataagaccccggcgccgccacc

SEQ ID NO: 25 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 26 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 27 - RNA, 83 nt, synthetic construct
  source 1..83: mol_type = other RNA
  misc_feature 14..18: note = Amino acids 14-18 may be absent.
  misc_feature 19: note = Amino acid 19 may be absent.
  misc_feature 22: note = Amino acid 22 may be absent.
  misc_feature 23..27: note = Amino acids 23-27 may be absent.
  misc_feature 81: note = Amino acid 81 may be absent.
ggaaatcgcaaaatttttgctctttttcgcgttagatttcttttagttttctykcaactagcaagctttttgttctcgccrcc

SEQ ID NO: 28 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 29 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 30 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 31 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 32 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 33 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 34 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 35 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 36 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 37 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 38 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 39 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 40 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 41 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 42 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 43 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 44 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 45 - RNA, 57 nt, synthetic construct
  source 1..57: mol_type = other RNA
ggaaataagagagaaaagaagagtaagaagaaatataagagccaaaaaaaaaaaacc

SEQ ID NO: 46 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 47 - RNA, 36 nt, synthetic construct
  source 1..36: mol_type = other RNA
ggactcactatttgttttcgcgcccagttgcaaaaa

SEQ ID NO: 48 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 49 - RNA, 119 nt, synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 50 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 51 - RNA, 170 nt, synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 52 - RNA, 141 nt, synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 53 - RNA, 185 nt, synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 54 - RNA, 187 nt, synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 55 - RNA, 170 nt, synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 56 - RNA, 141 nt, synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 57 - protein, 4 aa, synthetic construct
GGGS

(19) United States
(12) Patent Application Publication
(10) Pub. No.: US 2026/0158135 A1
(43) Pub. Date: Jun. 11, 2026

(54) VARIANT STRAIN-BASED CORONAVIRUS VACCINES AND USES THEREOF

(71) Applicant: ModernaTX, Inc., Cambridge, MA (US)

(72) Inventors: Mihir Metkar, Cambridge, MA (US); Guillaume Stewart-Jones, Cambridge, MA (US); Gwo-yu Chuang, Cambridge, MA (US)

(21) Appl. No.: 19/107,460

(22) PCT Filed: Aug. 31, 2023
(86) PCT No.: PCT/US2023/073247

§ 371 Date: Feb. 27, 2025

Related U.S. Application Data
(60) Provisional application No. 63/402,884, filed on Aug. 31, 2022.
(60) Provisional application No. 63/508,851, filed on Jun. 16, 2023.

(51) Int. Cl. A61K 39/215; A61K 39/00; A61P 37/04

(52) U.S. Cl. CPC A61K 39/215; A61P 37/04; A61K 2039/53; A61K 2039/545; A61K 2039/6018; A61K 2039/70; C12N 2770/20022; C12N 2770/20034; C12N 2770/20071

(57) ABSTRACT

The disclosure provides coronavirus mRNA vaccines, including vaccines directed against spike proteins of one or more variant strains of SARS-CoV-2, as well as methods of using the vaccines.

### CROSS-REFERENCE TO RELATED APPLICATIONS

This application is a National Stage application under 35 U.S.C. § 371 of International Application No. PCT/US2023/073247, filed on Aug. 31, 2023, which claims the benefit of priority from U.S. Provisional Application No. 63/402,884, filed on Aug. 31, 2022, and U.S. Provisional Application No. 63/508,851, filed Jun. 16, 2023, the contents and disclosures of which are incorporated herein by reference in their entireties.

### REFERENCE TO AN ELECTRONIC SEQUENCE LISTING

The contents of the electronic sequence listing (M137870252WO00-SEQ-JXV.xml; Size: 83,538 bytes; and Date of Creation: Aug. 29, 2023) is herein incorporated by reference in its entirety.

### BACKGROUND

Human coronaviruses are highly contagious enveloped, positive sense single-stranded RNA viruses of the Coronaviridae family. Two sub-families of Coronaviridae are known to cause human disease. The most important being the β-coronaviruses (betacoronaviruses). The β-coronaviruses are common etiological agents of mild to moderate upper respiratory tract infections. Outbreaks of novel coronavirus infections, such as the infections caused by a coronavirus initially identified from the Chinese city of Wuhan in December 2019; however, have been associated with a high mortality rate death toll. This recently identified coronavirus, referred to as Severe Acute Respiratory Syndrome Coronavirus 2 (SARS-CoV-2) (formerly referred to as a “2019 novel coronavirus,” or a “2019-nCoV”) has rapidly infected hundreds of thousands of people. The pandemic disease that the SARS-CoV-2 virus causes has been named by World Health Organization (WHO) as COVID-19 (Coronavirus Disease 2019). The first genome sequence of a SARS-CoV-2 isolate (Wuhan-Hu-1; USA-WA1/2020 isolate) was released by investigators from the Chinese CDC in Beijing on Jan. 10, 2020 at Virological, a UK-based discussion forum for analysis and interpretation of virus molecular evolution and epidemiology. The sequence was then deposited in GenBank on Jan. 12, 2020, having Genbank Accession number MN908947.1. Subsequently, a number of SARS-CoV-2 strain variants have been identified, some of which are more infectious than the SARS-CoV-2 isolate.

As of the time of worldwide emergency use authorization of the authorized SARS-CoV-2 nucleic acid-based vaccines, there is not yet a strategy for combatting the recently-discovered and later-emerging SARS-CoV-2 variants of concern (VOC). The continuing health problems and mortality associated with coronavirus infections, particularly the SARS-CoV-2 pandemic, are of tremendous concern internationally. The public health crisis caused by SARS-CoV-2 and its variants reinforces the importance of rapidly developing effective and safe vaccine candidates against these viruses.

There remains a need for development and evaluation of further COVID-19 vaccines against SARS-CoV-2 variants encoding the prefusion stabilized S protein of SARS-CoV-2 that incorporates mutations present in variants, including L18F, D80A, D215G, L242-244del, R246I, K417N, E484K, N501Y, D614G, A701V, A67V, Δ69-70, T95I, G142D/Δ143-145, Δ211/L212I, ins214EPE, G339D, S371L, S373P, S375F, N440K, G446S, S477N, T478K, E484A, Q493K, G496S, Q498R, Y505H, T547K, H655Y, N679K, P681H, N764K, D796Y, N856K, Q954H, N969K, L981F, T19I, L24(del), P25 (del), P26 (del), A27S, H69 (del), V70 (del), G142D, V213G, G339D, S371F, S373P, S375F, T376A, D405N, R408S, N440K, L452R, S477N, T478K, F486V, Q498R, Y505H, N764K, and any combination thereof. Additional vaccines are necessary to expand the breadth of coverage to multiple circulating variants as well as the ancestral wild-type virus that is still circulating globally.

### SUMMARY

A SARS-CoV-2 vaccine, mRNA-1273 (developed by Moderna Therapeutics), has been shown to elicit high viral neutralizing titers in Phase 1 trial human participants (Jackson et al, 2020; Anderson et al, 2020) and is highly efficacious in prevention of symptomatic COVID-19 disease and severe disease (Baden et al., 2020). However, the recent emergence of SARS-CoV-2 variants in the United Kingdom (B.1.1.7 lineage; alpha) and in South Africa (B.1.351 lineage; beta) have raised concerns due to their increased rates of transmission as well as their potential to circumvent immunity elicited by natural infection or vaccination (Volz et al., 2021; Tegally et al., 2020; Wibmer et al., 2021; Wang et al., 2021; Collier et al., 2021).

First detected in September 2020 in South England, the SARS-CoV-2 B.1.1.7 variant (alpha variant) has spread at a rapid rate and is associated with increased transmission and higher viral burden (Rambaut et al., 2020). This variant has seventeen mutations in the viral genome. Among them, eight mutations are located in the spike (S) protein, including 69-70 del, Y144 del, N501Y, A570D, P681H, T716I, S982A and D1118H. Two key features of this variant, the 69-70 deletion and the N501Y mutation in S protein, have generated concern among scientists and policy makers in the UK based on increased transmission and potentially increased mortality, resulting in further shutdowns. The 69-70 deletion is associated with reduced sensitivity to neutralization by SARS-CoV-2 human convalescent serum samples (Kemp et al, 2021). N501 is one of the six key amino acids interacting with ACE-2 receptor (Starr et al. 2020), and the tyrosine substitution has been shown to have increased binding affinity to the ACE-2 receptor (Chan et al., 2020).

The omicron BA.4 and BA.5 sub-variants date to January 2022 from South Africa. While BA.4 and BA.5 are two different Omicron sub-variants, they are usually discussed together for vaccine/immunization purposes, as they encode identical spike proteins. Early data from South Africa and genetic and epidemic surveillance in several countries indicated that BA.4/BA.5 had substantial growth advantage over other SARS-CoV-2 circulating strains. This advantage was likely driven by new mutations in BA.4/BA.5 spike that provided increased escape from pre-existing immunity in the populations acquired either via natural infection or vaccinations. In response, the European Centers for Disease Control and Prevention (ECDC) and the UK Health Security Agency (UKHSA) designated BA.4/BA.5 as Variants of Concern (VOC) in May 2022. BA.5 became dominant variant in Portugal in May 2022, while BA.4/BA.5 became dominant in the USA, France, UK, and Germany in June 2022.

In some aspects, messenger ribonucleic acid (mRNA) vaccine comprising: a first mRNA comprising a first open reading frame (ORF) encoding a first SARS-CoV-2 spike protein, wherein the SARS-CoV-2 spike protein comprises at least one of the following mutations relative to SEQ ID NO: 5: L24(del) or A27S, is provided.

In some embodiments, the first SARS-CoV-2 spike protein comprises an L24(del) mutation relative to SEQ ID NO: 5. In some embodiments, the first SARS-CoV-2 spike protein comprises an A27S mutation relative to SEQ ID NO: 5. In some embodiments, the first SARS-CoV-2 spike protein comprises L24(del) and A27S mutations relative to SEQ ID NO: 5.

In some embodiments, the first SARS-CoV-2 spike protein further comprises at least one of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N %9K. In some embodiments, the first SARS-CoV-2 spike protein further comprises the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N %9K.

In some embodiments, the first SARS-CoV-2 spike protein comprises an amino acid sequence having at least 98% identity to SEQ ID NO: 12. In some embodiments, the first ORF comprises a nucleotide sequence that has at least 89% identity to the nucleotide sequence of SEQ ID NO: 10. In some embodiments, the first ORF comprises a nucleotide sequence that has at least 99% identity to the nucleotide sequence of SEQ ID NO: 10.

In some embodiments, wherein the mRNA vaccine further comprises a second mRNA comprising a second ORF encoding a second SARS-CoV-2 spike protein. In some embodiments, the second SARS-CoV-2 spike protein is different from the first SARS-CoV-2 spike protein. In some embodiments, the amino acid sequence of the second SARS-CoV-2 spike protein is at least 95% identical to the amino acid sequence of the first SARS-CoV-2 spike protein. In some embodiments, the first mRNA and the second mRNA are present in the mRNA vaccine at a ratio of 1:1.

In some embodiments, the mRNA vaccine comprises 25 μg-75 μg of mRNA in total. In some embodiments, the mRNA vaccine comprises 50 μg of mRNA in total.

In some embodiments, the first mRNA and, optionally, the second mRNA, comprises a chemical modification. In some embodiments, the mRNA is fully chemically modified. In some embodiments, the chemical modification is 1-methylpseudouridine.

In some embodiments, the mRNA vaccine further comprises a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the ionizable amino lipid has the structure of Compound 1:

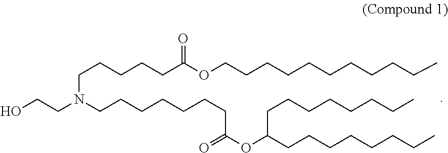 [SMILES: CCCCCCCCCCCOC(=O)CCCCCN(CCO)CCCCCCCC(=O)OC(CCCCCCCC)CCCCCCCC]

In some embodiments, the sterol is cholesterol or a derivative thereof. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

In some aspects, a method comprising administering to a subject a mRNA vaccine comprising a first mRNA comprising a first open reading frame (ORF) encoding a first SARS-CoV-2 spike protein, wherein the SARS-CoV-2 spike protein comprises at least one of the following mutations relative to SEQ ID NO: 5: L24(del) or A27S, is provided.

In some embodiments, first SARS-CoV-2 spike protein further comprises at least one of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K. In some embodiments, the first mRNA is fully chemically modified. In some embodiments, the mRNA vaccine further comprises a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid. In some embodiments, the ionizable amino lipid has the structure of Compound 1:

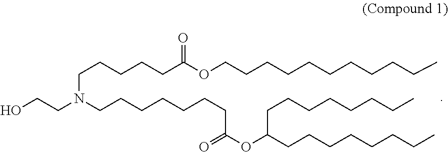 [SMILES: CCCCCCCCCCCOC(=O)CCCCCN(CCO)CCCCCCCC(=O)OC(CCCCCCCC)CCCCCCCC]

In some aspects, compositions (e.g. vaccines) comprising a nucleic acid encoding a SARS-CoV-2 antigen (e.g., an mRNA vaccine) are provided. A messenger ribonucleic acid (mRNA) vaccine may comprise a first mRNA comprising a first open reading frame (ORF) encoding a SARS-CoV-2 spike protein, wherein the ORF comprises a nucleotide sequence that has at least 99% identity to the nucleotide sequence of SEQ ID NO: 10.

In some embodiments, the ORF comprises SEQ ID NO: 10. In some embodiments, the first mRNA comprises a nucleotide sequence that has at least 99% identity to the nucleotide sequence of SEQ ID NO: 9. In some embodiments, the first mRNA comprises SEQ ID NO: 9.

In some embodiments, the mRNA vaccine further comprises a second mRNA comprising a second ORF encoding a SARS-CoV-2 spike protein, wherein the ORF comprises SEQ ID NO: 3. In some embodiments, the second mRNA comprises SEQ ID NO: 1.

In some aspects, a first mRNA comprising SEQ ID NO: 9 and a second mRNA comprising SEQ ID NO: 1 is provided.

In some embodiments, the first mRNA and the second mRNA are present in the mRNA vaccine at a ratio of 1:1.

In some embodiments, the mRNA vaccine comprises 25 μg-75 μg of mRNA in total. In some embodiments, the mRNA vaccine comprises 50 μg of mRNA in total.

In some embodiments, the first mRNA and, optionally, the second mRNA, comprises a chemical modification. In some embodiments, the mRNA is fully chemically modified. In some embodiments, the chemical modification is 1-methylpseudouridine.

In some embodiments, the mRNA vaccine further comprises a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the ionizable amino lipid has the structure of Compound 1:

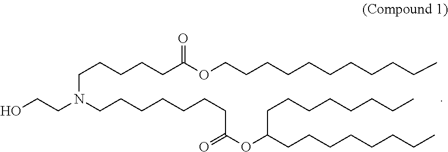 [SMILES: CCCCCCCCCCCOC(=O)CCCCCN(CCO)CCCCCCCC(=O)OC(CCCCCCCC)CCCCCCCC]

In some embodiments, the sterol is cholesterol or a derivative thereof. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

In some aspects, a method comprising administering to a subject a mRNA vaccine comprising a first mRNA comprising a first open reading frame (ORF) encoding a SARS-CoV-2 spike protein, wherein the ORF comprises a nucleotide sequence that has at least 99% identity to the nucleotide sequence of SEQ ID NO: 10 is provided.

In some embodiments, the ORF comprises SEQ ID NO: 10. In some embodiments, the first mRNA comprises a nucleotide sequence that has at least 99% identity to the nucleotide sequence of SEQ ID NO: 9. In some embodiments, the first mRNA comprises SEQ ID NO: 9.

In some embodiments, the mRNA vaccine further comprises a second mRNA comprising a second ORF encoding a SARS-CoV-2 spike protein, wherein the ORF comprises SEQ ID NO: 3. In some embodiments, the second mRNA comprises SEQ ID NO: 1.

In some aspects, a method comprising administering to a subject a mRNA vaccine comprising a first mRNA comprising SEQ ID NO: 9 and a second mRNA comprising SEQ ID NO: 1 is provided.

In some embodiments, the first mRNA and the second mRNA are present in the mRNA vaccine at a ratio of 1:1.

In some embodiments, the mRNA vaccine comprises 25 μg-75 μg of mRNA in total. In some embodiments, the mRNA vaccine comprises 50 μg of mRNA in total.

In some embodiments, the first mRNA and, optionally, the second mRNA, comprises a chemical modification. In some embodiments, the mRNA is fully chemically modified. In some embodiments, the chemical modification is 1-methylpseudouridine.

In some embodiments, the mRNA vaccine is administered at least about four months after a SARS-CoV-2 booster dose. In some embodiments, the mRNA vaccine is administered in an effective amount to induce an immune response specific for the protein encoded by the first mRNA and, optionally the protein encoded by the second mRNA.

In some embodiments, the mRNA vaccine further comprises a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid. In some embodiments, the ionizable amino lipid has the structure of Compound 1:

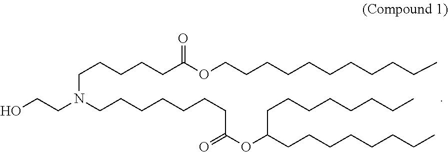 [SMILES: CCCCCCCCCCCOC(=O)CCCCCN(CCO)CCCCCCCC(=O)OC(CCCCCCCC)CCCCCCCC]

In some embodiments, the sterol is cholesterol or a derivative thereof. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

In some aspects, a method for identifying a dose of a vaccine, comprising, determining an immune response stimulation (IS) value of the measured immune response dynamics following vaccination (ID) with an mRNA vaccine, and determining an effective dose of the vaccine based on IS/ID model is described. Such a model allows one to make informed dose projections characterizing the mRNA vaccine response and projected outcomes in untested scenarios.

### BRIEF DESCRIPTION OF THE DRAWINGS

FIGS. 1A-1B show the geometric mean titer (GMT) of antibodies against Ancestral SARS-CoV-2 (D614G) (FIG. 1A) and Omicron BA.4/BA.5 (FIG. 1B) at baseline (pre-booster) and on Day 29 (28 days after injection) for subjects treated with mRNA-1273 or mRNA-1273.222. GMT values are shown at the head of each bar. Error bars indicate 95% confidence intervals.

FIGS. 2A-2B shows box-and-whisker plots for 50% inhibitory serum dilution (ID50) titer against Omicron BA.4/BA.5, BQ.1.1, XBB.1, and XBB.1.5 variants in subjects treated with mRNA-1273.222 at baseline (pre-booster) and on Day 29 (28 days after injection). Mean GMT value is shown above each box, and individual titers are shown as lines. Fold-change in mean GMT value from baseline to Day 29 is shown above each set for each variant. FIG. 2A shows box-and-whisker plots for subjects with no history of SARS-CoV-2 infection (n=40) and treated with mRNA-1273.222. FIG. 2B shows box-and-whisker plots for subjects with history of a previous SARS-CoV-2 infection (n=20) and treated with mRNA-1273.222.

FIG. 3 shows development of the immunostimulatory/immunodynamic (IS/ID) model. The left panel summarizes the data used for model development. The right panel summarizes subsequent evaluation steps of the base model.

FIG. 4 shows the IS/ID model, illustrating vaccine-mediated biological mechanisms, secretion of neutralizing antibodies (nAbs), and in vivo disposition of nAbs following mRNA-1273 administration. β_{AB}, transition rate from active B cells to memory B cells; β_{LL}, transition rate to LLPCs; β_{MB}, rate of entry into activated B-cell population; δ, B-cell activation rate; IS/ID, immunostimulatory/immunodynamic; k_{MAB}, antibody production rate; k_{cl}, antibody elimination rate; LLPC, long-lived plasma cell; MBₘₐₓ, maximum number of memory B cells; nAb, neutralizing antibody; R_{MB}, proliferation rate of memory B cells; μ_{LL}, death rate of LLPCs; μ_{MB}, death rate of memory B cells.

FIG. 5 shows distribution plots of Log pseudovirus neutralizing antibody infectious dose 50 titer (PSVN50) versus time by age group and mRNA-1273 dose. T represents timepoint.

FIG. 6 shows model-predicted geometric mean ratio (GMR) and observed GMR in different age groups. In the figure, CI, confidence interval; GMR, geometric mean ratio; m, months; y, years.

FIGS. 7A-7B shows correlations between nAb titers and model predictions. FIG. 7A shows correlation between observed nAB titers and individual predictions. FIG. 7B shows correlation between observed nAB titers and population predictions. In the figures, PSVN50, pseudovirus neutralizing antibody ID50 titer.

FIGS. 8A-8B shows the distribution of random effect on volume scaling factors. FIG. 8A shows the normal distribution assumption is preserved. FIG. 8B demonstrates that the distribution of random effect on volume scaling factor across age groups shows no bias.

FIGS. 9A-9E show the neutralizing antibody response following booster doses of PBS, mRNA-1273, mRNA-1273.214, and mRNA-1273.222. FIG. 9A is a schematic depicting the study design. Neutralizing antibody responses were measured pre- and post-boost against WA1/2020 (D614G) (FIG. 9B), the Delta variant (B.1.617.2) (FIG. 9C), the BA.1 variant (FIG. 9D), and the BA.5 variant (FIG. 9E). In FIGS. 9B-9E, the graphs, from left to right, are from treatment with PBS, mRNA-1273, mRNA-1273.214, and mRNA-1273.222.

FIGS. 10A-10C show viral loads in mice challenged with BA.5 after boosting with 0.25 μg mRNA-1273, mRNA-1273.214, mRNA-1273.222, or control (PBS) vaccines. Viral load was measured from lung samples (FIG. 10A), nasal turbinates (FIG. 10B), and nasal wash (FIG. 10C).

FIG. 11 shows binding antibody responses in BALB/c mince after a primary series vaccine.

FIGS. 12A-12B show neutralizing antibody responses in BALB/c mice after a primary series vaccine (day 35). FIG. 12A shows results from a VSV-based assay and FIG. 12B shows results from a lentivirus-based assay.

FIGS. 13A-13F show in vitro surface expression of the BA.4/BA.5 SARS-CoV-2 antigen after transfection of Expi293 cells with BA.4/BA.5 mRNA contained in mRNA-1273.222 of mRNA contained in mRNA-1283 (positive control), measured by flow cytometry at 48 hours. The frequency (FIG. 13A) and intensity (FIG. 13D) of CR3022-positive cells; the frequency (FIG. 13B) and intensity (FIG. 13E) of CC40.8-positive cells; and the frequency (FIG. 13C) and intensity (FIG. 13F) of hACE-2-positive cells were measured. In the figures, the groups (from left to right) are: 500 ng per 1 million cells and 100 ng per 1 million cells.

### DETAILED DESCRIPTION

Severe acute respiratory syndrome coronavirus 2 (SARS-CoV-2) is a newly emerging respiratory virus with high morbidity and mortality. SARS-CoV-2 has rapidly spread around the world compared with SARS-CoV, which appeared in 2002, and Middle East respiratory syndrome coronavirus (MERS-CoV), which emerged in 2012. The World Health Organization (WHO) reports that, as of March 2021, the current outbreak of COVID-19 has had over 120 million confirmed cases worldwide with more than 6.5 million deaths. New cases of COVID-19 infection are on the rise and are still increasing rapidly. It is thus crucial that a variety of safe and effective vaccines and drugs be developed to prevent and treat COVID-19 and reduce the serious impact that COVID-19 is having across the world. Vaccines and drugs made using a variety of modalities, and vaccines having improved safety and efficacy, are imperative. There remains a need to accelerate the advanced design and development of vaccines and therapeutic drugs against coronavirus disease and in particular coronavirus 2019 (COVID-19).

Severe acute respiratory syndrome coronavirus 2 (SARS-CoV-2) was identified as the etiological agent of a novel pneumonia that emerged in December 2019 in China (Lu H. et al. (2020) J Med Virol. April; 92(4):401-402.). Soon after, the virus caused an outbreak in China and spread to the world. According to the analysis of genomic structure of SARS-CoV-2, it belongs to β-coronaviruses (CoVs) (Chan et al. 2020 Emerg Microbes Infect.; 9(1):221-236).

Subsequently, a number of SARS-CoV-2 variant strains have emerged and have predominated in particular initial geographic areas. However, some variants that quickly predominate in one geographic area can spread rapidly around the globe. These variants are known as variants of concern (VOC). There are concerns that these variants as well as other circulating strains and any future variants may further mutate to avoid neutralization by existing vaccines and therapeutic modalities such as antibodies. In this way, the SARS-CoV-2 variants, and any other emerging mutant SARS-CoV-2 strains, are an international health concern.

The threat of emerging mutant strains of viruses presents a significant challenge to vaccine development. mRNA vaccines and vaccine protocols provide a significant advance in combatting the emerging viral strains that pose a global health concern. In some embodiments, vaccines and vaccine protocols with broad viral neutralization capabilities may reduce the threat of infection from more than one strain of virus, through single or multiple administrations of the same or different combinations of antigens from different strains. For instance, in some embodiments, vaccination strategies comprise “primary series” of vaccinations and subsequent boost(s) of SARS-CoV-2 2P stabilized spike protein antigen. The primary series (also referred to as initial, original or first vaccine, vaccination) involves the administration of one or more vaccines (e.g. two vaccine administrations) of the SARS-CoV-2 2P stabilized spike protein antigen from the originally identified strain of SARS-CoV-2. The primary series of vaccine may be an mRNA vaccine encoding an antigen having an amino acid sequence of SEQ ID NO: 5. A subsequent booster or booster series of vaccines is then administered, for instance, shortly after the original vaccine or at a significantly later time in the vaccination protocol (e.g., after neutralizing antibody titers have dropped or after approval of a new strain vaccine.

In some aspects, emerging SARS-CoV-2 variant strains are used to design mRNA “boost” as a supplement to prior administered SARS-CoV-2 vaccines and includes traditional boosts, seasonal boosts and pandemic shift boosts. A boost, as used herein, refers to any subsequent dose. A traditional boost is a second dose of an antigen administered to a subject following a period of time, such as 21-28 days or even 2 weeks to 6 months. The traditional boost involves the administration of the same antigen representing the same virus strain to the subject in order to generate a robust immune response against that viral strain and optionally other variant strains.

During a pandemic or endemic, emerging viral strains may develop which are not effectively susceptible to neutralization with a vaccine designed against the original strain. In particular, SARS-CoV-2 emerging viral strains appear to arise through radial evolution; that is, with a variety of different mutations, as compared to linear evolution, in which mutations accumulate upon one another as the virus evolves. In such instances, a pandemic shift boost may be used to provide immune protection against emerging viral strains. A pandemic shift boost is a subsequent vaccine which is administered to a subject following a complete course of a first vaccine. The complete course of the first vaccine may comprise one or more administrations of the first vaccine. The pandemic shift boost is comprised of a vaccine that includes an antigen which is derived from a variant viral strain that has emerged during a pandemic or endemic of the viral infection. The pandemic shift boost may be administered at any time following the administration of the first vaccine. The first vaccine may be a vaccine against the originally detected strain of the virus, a combination of the original strain of the virus and variant strain(s) of the virus, or variant strains of the virus, as long as the pandemic shift boost comprises a vaccine against a different variant strain of the virus from the first vaccine.

Additionally variant viral strains of SARS-CoV-2 may emerge at times outside of a pandemic or endemic. These strains may emerge, for instance, seasonally. Such variant strains may be used to design seasonal SARS-CoV2 vaccines which as delivered as a seasonal boost. A seasonal boost is a subsequent vaccine which is administered to a subject following a complete course of a first vaccine which happens outside of a pandemic or endemic, as variant strains arise. Viral surveillance methods are used in the design of traditional vaccines. However, due to the slow development time of traditional vaccines, the antigen design decisions are often made so far in advance that the vaccine does not match the viral strains circulating when the vaccines are administered. During the development period the viruses may mutate, or other strains may become more prevalent, such that the traditional vaccines become less effective. The traditional vaccines cannot adapt because they are already in production, and it would take additional time to design and manufacture a new vaccine. In contrast, mRNA vaccines are able to overcome these challenges. They can be produced in a matter of weeks, so that they can be designed against the coronaviruses circulating closer to the inoculation date. For instance, a seasonal or annual coronavirus vaccination program can be developed that rapidly develops a coronavirus vaccine in response to viral strains circulating at the time of vaccination. That is, it is thought that prediction of the viruses closer to a coronavirus season or other outbreak will be more accurate than predictions from several months before the season or the outbreak begins, and therefore mRNA vaccines may be more effective because they are designed to target circulating viruses closer to the coronavirus season or scheduled inoculations. Thus, in exemplary aspects, vaccines may be designed to combat seasonal coronavirus strains, and as such are vaccines for use in an upcoming or forthcoming Northern hemisphere season or Southern hemisphere season. Based on an understanding of circulating coronaviruses at a given point in time, the vaccines are designed to combat such viruses as they are predicted to be those that will be circulating or prevalent in the upcoming or forthcoming virus season. The mRNA vaccines can be designed in a matter of days and a recent vaccine developed by applicant preceded from design to manufactured vaccine in just over 5 weeks. Data can be captured and analyzed as to what viruses are circulating and with what prevalence, much closer to the start of an inoculation program such as seasonal vaccination.

A key protein on the surface of coronavirus, including the SARS-CoV-2 and mutant strains, is the Spike (S) protein. A stabilized version of the spike protein having a two proline (2P) mutation relative to wild-type SARS-CoV-2 has been developed and has an amino acid sequence of SEQ ID NO: 5. The 2P stabilized spike antigen is a full length spike protein including the 2Ps. In some aspects, vaccination protocols comprise various vaccines of full length 2P stabilized spike protein from the original SARS-CoV-2 strain and/or emerging variant SARS-CoV-2 strains, wherein each antigen includes the 2P mutation.

The inventors have designed a variety of mRNA constructs. When formulated in appropriate delivery vehicles, mRNA encoding a 2P stabilized version of the spike antigen of emerging variant strains are capable of inducing a strong immune response against SARS-CoV-2, thus producing effective and potent mRNA vaccines/boosters to provide the diversity essential to eradicating the original virus as well as subsequent strains. Intramuscular administration of the mRNA encoding various Spike protein antigens in an LNP, in particular, Spike protein subunit and domain antigens, results in delivery of the mRNA to immune tissues and cells of the immune system where it is rapidly translated into proteins antigens. Other immune cells, for example, B cells and T cells, are then able to recognize and mount an immune response against the encoded protein and ultimately create a long-lasting protective response against the coronavirus. Low immunogenicity, a drawback in protein vaccine development due to poor presentation to the immune system or incorrect folding of the antigens, can be avoided through the use of highly effective mRNA vaccine compositions, which encode, in some aspects, spike protein, subunits and/or domains thereof.

Due to the constant evolving nature of viruses, scientists continuously monitor the sequences and strains of viruses circulating in humans. These various circulating strains may be used to design boosts or individual vaccines or, additionally, to design multivalent mRNA vaccines. Viral surveillance can be used to provide annual or seasonal (or other scheduled) information to select the precise virus strains to be used as the basis of mRNA vaccines. Once circulating strains are identified, the composition of a vaccine that targets two or three (or more) most representative virus types in circulation can be developed based on those strains. This exercise of adding antigens from new strains to the vaccine can be repeated on an annual basis or other time frame as required to maintain viral immunity in the population. As used herein, “population” or “subject population” refers to the global population, a regional population, or a national population. For example, a regional population may refer to a geographically distinct population (e.g., hemisphere, continent) or a region of a country, as some new strains may be more prevalent in certain regions of the world, continents, or countries. In some embodiments, the subject population is a national population (e.g., the population of the United States). In some embodiments, mRNA vaccines encode multiple antigens from multiple circulating strains in a single lipid nanoparticle (LNP). The mRNA vaccines comprise, in some embodiments, a combination of at least two antigens, each derived from a unique strain of coronavirus.

In some aspects, compositions (e.g., mRNA vaccines) elicit potent neutralizing antibodies against coronavirus antigens in subjects. Such a composition can be administered to seropositive or seronegative subjects. A seropositive subject may be naïve and not have antibodies that react with SARS-CoV-2. A seronegative subject may have preexisting antibodies to SARS-CoV-2 because they have previously had an infection with SARS-CoV-2 or may have previously been administered a dose of a vaccine (e.g., an mRNA vaccine) that induces antibodies against SARS-CoV-2. In some embodiments, a composition includes mRNA encoding at least one (e.g., one, two, or more) coronavirus antigens, such as SARS-CoV-2 antigens from different SARS-CoV-2 mutant strains (also referred to herein as variants). In some embodiments, the mRNA vaccine comprises multiple mRNAs encoding SARS-CoV-2 antigens from different variants in a single lipid nanoparticle. In some embodiments, the mRNA vaccine comprises an mRNA encoding a SARS-CoV-2 antigen comprising one or more mutations from at least two different SARS-CoV-2 variants (e.g., encoding a combination of the mutations and/or deletions found in the BA.4/BA.5 variants).

A variant of concern, Omicron (B.1.1.529), having multiple Spike protein mutations was detected initially in Botswana. The mutations observed in the variant include those found in the Delta variant that are believed to increase transmissibility and mutations, and those seen in the Beta and Delta variants that are believed to promote immune escape. In particular, the genome of the Omicron variant encodes a Spike protein having the following mutations: A67V, Δ69-70, T95I, G142D/Δ143-145, Δ211/L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, and L981F.

Two sub-variants (sublineages) of Omicron have since occurred: BA.4 and BA.5. Similar to Omicron, BA.4 and BA.5 comprise mutations that are thought to increase transmissibility and to promote immune escape. Numerous BA.4 and BA.5 spike protein haplotypes have been discovered, having a combination of the following mutations: T19I, L24(del), P25 (del), P26 (del), A27S, H69 (del), V70 (del), G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K, V3G, T761, and N658S.

These exemplary strains and other newly emerging strains are candidates for various methods and formulations. mRNA encoding antigens from these and other coronavirus strains have been designed for mRNA vaccines.

In some embodiments, mRNA vaccines may be administered as a booster, that is, a dose administered after a prime or priming immunization. In some embodiments, the booster and the prime or priming immunization comprise the same mRNA or mRNAs. In other embodiments, the booster and the prime or priming immunization comprise different mRNA or mRNAs. In other embodiments multiple mRNA vaccines encoding different antigens (each directed at a strain or multiple strains) may be administered together or in tandem to provide a wide spectrum neutralization platform against multiple coronavirus strains. Combinations of mRNAs have been demonstrated to be particularly effective in vivo and, quite surprisingly, even producing robust immune responses against variant strains that are not part of the vaccine. For instance, it was shown that when a multivalent mRNA-vaccine was administered as a booster it elicited robust and comparable neutralizing titers against both variant strains of the viruses not included in the prime or boost.

The genome of SARS-CoV-2 is a single-stranded positive-sense RNA (+ssRNA) with the size of 29.8-30 kb encoding about 9860 amino acids (Chan et al. 2020, supra; Kim et al. 2020 Cell, May 14; 181(4):914-921.e10.). SARS-CoV-2 is a polycistronic mRNA with 5′-cap and 3′-poly-A tail. The SARS-CoV-2 genome is organized into specific genes encoding structural proteins and nonstructural proteins (Nsps). The order of the structural proteins in the genome is 5′-replicase (open reading frame (ORF)1/ab)-structural proteins [Spike (S)-Envelope (E)-Membrane (M)-Nucleocapsid (N)]-3′. The genome of coronaviruses includes a variable number of open reading frames that encode accessory proteins, nonstructural proteins, and structural proteins (Song et al. 2019 Viruses; 11(1): p. 59). Most of the antigenic peptides are located in the structural proteins (Cui et al. 2019 Nat. Rev. Microbiol.; 17(3):181-192). Spike surface glycoprotein (S), a small envelope protein (E), matrix protein (M), and nucleocapsid protein (N) are four main structural proteins. Since S-protein contributes to cell tropism and virus entry and also it is capable to induce neutralizing antibodies (nAb) and protective immunity, it can be considered one of the most important targets in coronavirus vaccine development among all other structural proteins. Moreover, amino acid sequence analysis has shown that S-protein contains conserved regions among the coronaviruses, which may be the basis for universal vaccine development.

In some aspects, compositions, e.g., vaccine compositions, feature nucleic acids, in particular, mRNAs, designed to encode an antigen of interest, e.g., an antigen derived from a betacoronavirus structural protein, in particular, antigens derived from SARS-CoV-2 Spike protein. These compositions, e.g., vaccine compositions, do not comprise antigens per se, but rather comprise nucleic acids, in particular, mRNA(s) that encode antigens or antigenic sequences once delivered to a cell, tissue or subject. Delivery of nucleic acids, in particular mRNA(s) is achieved by formulating said nucleic acids in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs.

Antigens, as used herein, are proteins capable of inducing an immune response (e.g., causing an immune system to produce antibodies against the antigens). In some aspects, vaccines provide a unique advantage over traditional protein-based vaccination approaches in which protein antigens are purified or produced in vitro, e.g., recombinant protein production technologies. In some aspects, vaccines feature mRNA encoding the desired antigens, which when introduced into the body, i.e., administered to a mammalian subject (for example a human) in vivo, cause the cells of the body to express the desired antigens. In order to facilitate delivery of the mRNAs to the cells of the body, the mRNAs are encapsulated in lipid nanoparticles (LNPs). Upon delivery and uptake by cells of the body, the mRNAs are translated in the cytosol and protein antigens are generated by the host cell machinery. The protein antigens are presented and elicit an adaptive humoral and cellular immune response. Neutralizing antibodies are directed against the expressed protein antigens and hence the protein antigens are considered relevant target antigens for vaccine development. Herein, use of the term “antigen” encompasses immunogenic proteins and immunogenic fragments (an immunogenic fragment that induces (or is capable of inducing) an immune response to a (at least one) SARS-CoV-2 variant), unless otherwise stated. It should be understood that the term “protein” encompasses peptides and the term “antigen” encompasses antigenic fragments. Other molecules may be antigenic such as bacterial polysaccharides or combinations of protein and polysaccharide structures, but for these vaccines, suitable antigens include viral proteins, fragments of viral proteins, and designed and/or mutated proteins derived from SARS-CoV-2.

Many proteins have a quaternary or three-dimensional structure, which consists of more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

In structural and molecular biology, identical, homologous or similar subunits or domains can help to classify newly identified or novel proteins, as was done immediately upon publication of the SARS-CoV-2 viral genomic sequence.

As used herein, the term antigen is distinct from the term “epitope” which is a substructure of an antigen, e.g., a polypeptide, such as 7-10 amino acids, or carbohydrate structure, which may be recognized by an antigen binding site but is insufficient to induce an immune response. The art describes protein antigens that are delivered to subjects or immune cells in isolated form, e.g., isolated protein, polypeptide or peptide antigens, however, the design, testing, validation, and production of protein antigens can be costly and time-consuming, especially when producing proteins at large scale. By contrast, mRNA technology is amenable to rapid design and testing of mRNA constructs encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs are expressed by the cells of the subject, e.g., are expressed by the human body, and thus the subject, e.g., the human body, serves as the “factory” to produce the antigens which, in turn, elicits the desired immune response.

Compositions may include an RNA or multiple RNAs encoding two or more antigens of the same or different viral strains (i.e. combination vaccines). In some embodiments, combination vaccines include RNA encoding one or more coronavirus antigens and one or more antigen(s) of a different organism. Thus, vaccines may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus (e.g., COVID-19).

In some embodiments, the second or subsequent circulating SARS-CoV-2 virus is an immunodominant antigen from an emerging strain. An immunodominant antigen of an emerging strain is assessed with respect to the strain from which the antigen is derived, relative to a different strain of the virus, such as the original strain or other variant thereof. An immunodominant antigen of the emerging strain induces a stronger immune response against the emerging strain than against the different strain. In some embodiments, an immunodominant antigen of the emerging strain is more infective than a different strain of the virus, such as the original strain or other variant thereof.

### Encoded Coronavirus Spike (S) Protein Antigens

The envelope spike (S) proteins of known betacoronaviruses determine the virus host tropism and entry into host cells. Coronavirus spike (S) protein is a choice antigen for the vaccine design as it can induce neutralizing antibodies and protective immunity. S protein is critical for SARS-CoV-2 infection. The organization of the S protein is similar among betacoronaviruses, such as SARS-CoV-2, SARS-CoV, MERS-CoV, HKU1-CoV, MHV-CoV and NL63-CoV.

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids.

SARS-CoV-2 spike (S) protein is a choice antigen for the vaccine design as it can induce neutralizing antibodies and protective immunity. mRNAs are designed to produce SARS-CoV-2 Spike proteins (i.e., encode Spike proteins such that Spike protein is expressed when the mRNA is delivered to a cell or tissue, for example a cell or tissue in a subject), as well as antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. In some embodiments, the Spike protein is a stabilized Spike protein, for example, the Spike protein is stabilized by two proline substitutions (a 2P mutation).

In some embodiments, the Spike protein is from a different virus strain. A strain is a genetic variant of a microorganism (e.g., a virus). New viral strains can be created due to mutation or swapping of genetic components when two or more viruses infect the same cell in nature, for example, by antigenic drift or antigenic shift.

Antigenic drift is a kind of genetic variation in viruses, arising by the accumulation of mutations in the virus genes that code for virus-surface proteins that host antibodies recognize. This results in a new strain of virus particles that is not effectively inhibited by the antibodies that prevented infection by previous strains. This makes it easier for the changed virus to spread throughout a partially immune population.

Antigenic shift is the process by which two or more different strains of a virus, or strains of two or more different viruses, combine to form a new subtype having a mixture of the surface antigens of the two or more original strains. The term is often applied specifically to influenza, as that is the best-known example, but the process is also known to occur with other viruses. Antigenic shift is a specific case of reassortment or viral shift that confers a phenotypic change. Antigenic shift is contrasted with antigenic drift, which is the natural mutation over time of known strains of a virus which may lead to a loss of immunity, or in vaccine mismatch. Antigenic shift is often associated with a major reorganization of viral surface antigens, resulting in a reassortment change the virus's phenotype drastically.

A virus strain as used herein is a genetic variant or of a virus that is characterized by a differing isoform of one or more surface proteins of the virus. In the case of SARS-CoV-2, for example, a different amino acid sequence in the SARS-CoV-2 spike protein where the immune response in an individual to the new strain is less effective than to the strain used to immunize or first infect the individual. A new virus strain may arise from natural mutation or a combination of natural mutation and immune selection due to an ongoing immune response in an immunized or previously infected individual. A new virus strain can differ by one, two, three or more amino acid mutations in regions of the spike protein responsible for a viral function such as receptor binding or viral fusion with a target cell. A spike protein from a new strain may differ from the parental strain by as much as 90%, 91%, 92%, 93%, 94%, 95%, %%, 97%, 98%, or 99% identity at the amino acid level.

A natural virus strain is a variant of a given virus that is recognizable because it possesses some “unique phenotypic characteristics” that remain stable (e.g., stable and heritable biological, serological, and/or molecular characters) under natural conditions. Such “unique phenotypic characteristics” are biological properties different from the compared reference virus, such as unique antigenic properties, host range (e.g., infecting a different kind of host), symptoms of disease caused by the strain, different type of disease caused by the strain (e.g., transmitted by different means), etc. A “unique phenotypic characteristic” can be detected clinically (e.g., clinical manifestations detected in a host infected with the strain) or within a comparative animal experiment in which a researcher skilled in the art of virology can distinguish between the reference control virus-infected animal and the animal infected with the alleged new strain, without knowing which animal received which virus and without having any information about the differences between the two viruses. Importantly, a virus variant with a simple difference in genome sequence is not a separate strain if there is no recognizable distinct viral phenotype. The extent of genomic sequence variation is irrelevant for the classification of a variant as a strain since a distinct phenotype sometimes arises from few mutations.

As an example, in some embodiments, the mRNA encodes an antigen from at least one virus strain variant or comprises mutations from at least one virus strain that is not wild-type SARS-CoV-2. Table 1, below, presents examples of Spike protein mutations in SARS-CoV-2 variants.

TABLE 1
Spike mutations in SARS-CoV-2 variants

| Variant | |
|---|---|
| Name | Amino Acid Changes in Spike |
| B.1.1.529 | A67V, Δ69-70, T95I, G142D/Δ143-145, Δ211/L212I, |
| (Omicron | ins214EPE, G339D, S371L, S373P, S375F, K417N, N440K, |
| variant; | G446S, S477N, T478K, E484A, Q493K, G496S, Q498R, |
| Botswana) | N501Y, Y505H, T547K, D614G, H655Y, N679K, P681H, |
| | N764K, D796Y, N856K, Q954H, N969K, L981F |
| BA.4/ | T19I L24- P25- P26- A27S H69- V70- G142D V213G |
| BA.5 | G339D S371F S373P S375F T376A D405N R408S K417N |
| | N440K L452R S477N T478K E484A F486V Q498R N501Y |
| | Y505H D614G H655Y N679K P681H N764K D796Y |
| | Q954H N969K |

In exemplary embodiments, a Spike protein, e.g., an encoded Spike protein antigen, has the amino acid sequence set forth in any one of SEQ ID NOs: 5, 8, and 12. In other embodiments, a Spike protein, e.g., an encoded Spike protein antigen, has no greater than 100, no greater than 90, no greater than 80, no greater than 70, no greater than 60, no greater than 50, no greater than 40, no greater than 30, no greater than 20, no greater than 10, or no greater than 5 amino acid substitutions and/or deletions as compared to (when aligned with) a Spike protein having the amino acid sequence as set forth in any one of SEQ ID NOs: 5, 8, and 12. Where minor variations are made in encoded Spike protein sequences, the variant preferably has the same activity as the reference Spike protein sequence and/or has the same immune specificity as the reference Spike protein, as determined for example, in immunoassays (e.g., enzyme-linked immunosorbent assays (ELISA assays).

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, specifically angiotensin-converting enzyme 2 (ACE2) receptors, whereas the S2 subunit, which is the most conserved component of the S protein, will be responsible for fusing the envelope of the virus with the host cell membrane. (See e.g., Shang et al., PLoS Pathog. 2020 March; 16(3):e1008392.). Each monomer of trimeric S protein trimer contains the two subunits, S1 and S2, mediating attachment and membrane fusion, respectively. As part of the infection process in vivo, the two subunits are separated from each other by an enzymatic cleavage process. S protein is first cleaved by furin-mediated cleavage at the S1/S2 site in infected cells. In vivo, a subsequent serine protease-mediated cleavage event occurs at the S2′ site within S1. In SARS-CoV2, the S1/S2 cleavage site is at amino acids 676—TQTNSPRRAR/SVA—688 (SEQ ID NO: 15). The S2′ cleavage site is at amino acids 811—KPSKR/SFI—818 (SEQ ID NO: 16).

As used herein, for example in the context of designing SARS-CoV-2 S protein antigens encoded by nucleic acids, e.g., mRNAs, the term “S1 subunit” (e.g., S1 subunit antigen) refers to the N-terminal subunit of the Spike protein beginning at the S protein N-terminus and ending at the S1/S2 cleavage site whereas the term “S2 subunit” (e.g., S2 subunit antigen) refers to the C-terminal subunit of the Spike protein beginning at the S1/S2 cleavage site and ending at the C-terminus of the Spike protein. As described supra, the skilled artisan will understand that, while an essentially full length or complete Spike protein S1 or S2 subunit may be necessary for receptor binding or membrane fusion, respectively, a certain amount of variation in S1 or S2 structure and/or sequence is tolerated when seeking primarily to elicit an immune response against Spike protein subunits. For example, minor truncation, e.g., of one to a few, possibly up to 4, 5, 6, 7, 8, 9 or 10 amino acids from the N- or C-terminus of the encoded subunit, e.g., encoded S1 or S2 protein antigens, may be tolerated without changing the antigenic properties of the protein. Likewise, variation (e.g., conservative substitution) of one to a few, possibly up to 4, 5, 6, 7, 8, 9 or 10 amino acids (or more) of the encoded Spike protein subunits, e.g., encoded S1 or S2 protein antigen, may be tolerated without changing the antigenic properties of the protein(s). In exemplary embodiments, a Spike protein, e.g., an encoded Spike protein antigen, has the amino acid sequence set forth in any one of SEQ ID NOs: 5, 8, and 12.

In some embodiments, the mRNA vaccine encodes an antigen having at least one of the following mutations relative to the SARS-CoV-2 S protein of SEQ ID NO: 5 (2P mutation version of WT): T19I, L24(del), P25 (del), P26 (del), A27S, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K. In some embodiments, the mRNA encodes an antigen having 2, 3, 4, 5, 6, 7, 8, 9, 10, 11, 12, 13, 14, 15, 16, 17, 18, 19, 20, 21, 22, 23, 24, 25, 26, 27, 28, or 29 of the mutations listed. In some embodiments, the mRNA encodes an antigen that has one or more deletions relative to the SARS-CoV-2 S protein of SEQ ID NO: 5. Exemplary deletions include, but are not limited to, positions, 24, 25, 26, 69, and 70. In some embodiments, the mRNA encodes an antigen having 1, 2, 3, 4, or 5 deletions. In some embodiments, the mRNA encoding an antigen has 1, 2, 3, 4, or 5 deletions, and 1, 2, 3, 4, 5, 6, 7, 8, 9, 10, 11, 12, 13, 14, 15, 16, 17, 18, 19, 20, 21, 22, 23, 24, 25, 26, 27, 28, or 29 mutations or any combination thereof.

In some embodiments, compositions (e.g. vaccines) comprise 1, 2, 3, 4, 5, or 6 mRNAs encoding different antigens, wherein each antigen comprises at least one mutation and/or at least one deletion. In some embodiments, compositions (e.g. mRNA vaccines) further comprise an mRNA encoding a wild-type SARS-CoV-2 S protein antigen or the antigenic fragment thereof. In some embodiments, compositions (e.g. mRNA vaccines) comprise a lipid nanoparticle (e.g. a lipid nanoparticle comprises 1, 2, 3, 4, 5, or 6 mRNAs encoding different antigens).

In some aspects, compositions (e.g. mRNA vaccines) comprise at least: a first mRNA comprising a first open reading frame (ORF) encoding a first SARS-CoV-2 spike antigen of a first SARS-CoV-2 virus wherein the first SARS-CoV-2 spike antigen has an amino acid sequence of SEQ ID NO: 5 or an amino acid sequence with at least one amino acid mutation, deletion, or both amino acid mutation and deletion with respect to a protein of SEQ ID NO: 5 and a second mRNA comprising a second ORF encoding a second SARS-CoV-2 spike antigen of a second SARS-CoV-2 virus wherein the second SARS-CoV-2 spike antigen has an amino acid sequence with at least one amino acid mutation, deletion, or both amino acid mutation and deletion with respect to a protein of SEQ ID NO: 5 (e.g., SEQ ID NO: 12).

In some embodiments, compositions (e.g. mRNA vaccines) comprise a first mRNA comprising a first ORF encoding a first SARS-CoV-2 spike antigen. In some embodiments, compositions (e.g. mRNA vaccines) comprise a first mRNA comprising a first ORF encoding a first SARS-CoV-2 spike antigen, wherein the first SARS-CoV-2 spike protein comprises at least one of the following mutations relative to SEQ ID NO: 5: L24(del) or A27S. In some embodiments, compositions (e.g. mRNA vaccines) comprise: a first mRNA comprising a first ORF encoding a first SARS-CoV-2 spike antigen, wherein the first SARS-CoV-2 spike antigen comprises an L24(del) mutation relative to SEQ ID NO: 5. In some embodiments, compositions (e.g. mRNA vaccines) comprise: a first mRNA comprising a first ORF encoding a first SARS-CoV-2 spike antigen, wherein the first SARS-CoV-2 spike antigen comprises an A27S mutation relative to SEQ ID NO: 5. In some embodiments, compositions (e.g. mRNA vaccines) comprise: a first mRNA comprising a first ORF encoding a first SARS-CoV-2 spike antigen, wherein the first SARS-CoV-2 spike antigen comprises an L24(del) and A27S mutation relative to SEQ ID NO: 5.

In some embodiments, a first SARS-CoV-2 spike antigen further comprises at least one of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K. In some embodiments, a first SARS-CoV-2 spike antigen further comprises at least two, at least three, at least four, at least five, at least six, at least seven, at least eight, at least 9, at least 10, at least 11, at least 12, at least 13, at least 14, at least 15, at least 16, at least 17, at least 18, at least 19, at least 20, at least 21, at least 22, at least 23, at least 24, or at least 25 of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K. In some embodiments, a first SARS-CoV-2 spike antigen further comprises the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K.

In some embodiments, compositions (e.g. mRNA vaccines) comprise a second mRNA comprising a second ORF encoding a second SARS-CoV-2 spike antigen. In some embodiments, compositions (e.g. mRNA vaccines) comprise a second mRNA comprising a second ORF encoding a second SARS-CoV-2 spike antigen, wherein the second SARS-CoV-2 spike antigen is different from the first SARS-CoV-2 spike antigen. In some embodiments, the amino acid sequence of the second SARS-CoV-2 spike antigen is at least 70%, 71%, 72%, 73%, 74%, 75%, 76%, 77%, 78%, 79%, 80%, 81%, 82%, 83%, 84%, 85%, 86%, 87%, 885, 89% 90%, 91%, 92%, 93%, 94%, 95%, 96%, 97%, 98%, 99%, or identical to the amino acid sequence of the first SARS-CoV-2 spike antigen.

In some embodiments, the first SARS-CoV-2 virus is a first circulating SARS-CoV-2 virus. In some embodiments, the second SARS-CoV-2 virus is a second circulating SARS-CoV-2 virus. “Circulating viruses”, as used herein, refers to viruses that have been in circulation for 1 month, 2 months, 3 months, 4 months, 5 months, 6 months, 7 months, 8 months, 9 months, 10 months, 11 months, a portion of a year, 1 year, 1.5 years, 2 years, 3 years, or longer.

In some embodiments, the first and second mRNAs are present in the composition in a 1:1, 1:2, 1:3, 1:4, 1:5, 1:6, 1:7, 1:8, 1:9, or 1:10 ratio. In another embodiment, the first and second mRNAs are present in the composition in a 2:1, 3:1, 4:1, 5:1, 6:1, 7:1, 8:1, 9:1, or 10:1 ratio. In some embodiments, the first and second mRNA are present in the composition in a 1:1 ratio.

In some embodiments, the first mRNA encodes SEQ ID NO: 20 (2P mutation version of WT) and the second mRNA encodes SEQ ID NO: 12.

In some embodiments, the composition further comprises a third mRNA encoding a third SARS-CoV-2 spike antigen of a third SARS-CoV-2 virus, wherein the third SARS-CoV-2 spike antigen has an amino acid sequence with at least one amino acid mutation with respect to a protein sequence of SEQ ID NO: 20.

In some embodiments, the composition further comprises a fourth mRNA encoding a fourth SARS-CoV-2 spike antigen of a fourth SARS-CoV-2 virus, wherein the fourth SARS-CoV-2 spike antigen has an amino acid sequence with at least one amino acid mutation with respect to a protein of SEQ ID NO: 20.

In some embodiments, the composition further comprises a fifth mRNA encoding a fifth SARS-CoV-2 spike antigen of a fifth SARS-CoV-2 virus, wherein the fifth SARS-CoV-2 spike antigen has an amino acid sequence with at least one amino acid mutation with respect to a protein of SEQ ID NO: 20.

In some embodiments, the composition further comprises a sixth mRNA encoding a sixth SARS-CoV-2 spike antigen of a sixth SARS-CoV-2 virus, wherein the sixth SARS-CoV-2 spike antigen has an amino acid sequence with at least one amino acid mutation with respect to a protein of SEQ ID NO: 20.

In some embodiments, the first and second antigens are antigens of the spike protein. In some embodiments, the third, fourth, fifth, and/or sixth antigens are antigens of the spike protein.

Exemplary sequences of the coronavirus antigens and the RNA encoding the coronavirus antigens (e.g., SARS-CoV-2 variant antigens) of mRNA vaccines are provided in Table 3. In some embodiments, the mRNA vaccines comprise a sequence that has at least 70%, 71%, 72%, 73%, 74%, 75%, 76%, 77%, 78%, 79%, 80%, 81%, 82%, 83%, 84%, 85%, 86%, 87%, 885, 89% 90%, 91%, 92%, 93%, 94%, 95%, 96%, 97%, 98%, or 99% identity to a sequence selected from SEQ ID NOs: 3, 7, and 10. In some embodiments, the mRNA vaccines comprise a sequence that has at least 70%, 71%, 72%, 73%, 74%, 75%, 76%, 77%, 78%, 79%, 80%, 81%, 82%, 83%, 84%, 85%, 86%, 87%, 885, 89% 90%, 91%, 92%, 93%, 94%, 95%, 96%, 97%, 98%, or 99% identity to SEQ ID NO: 3. In some embodiments, the mRNA vaccines comprise a sequence that has at least 70%, 71%, 72%, 73%, 74%, 75%, 76%, 77%, 78%, 79%, 80%, 81%, 82%, 83%, 84%, 85%, 86%, 87%, 885, 89% 90%, 91%, 92%, 93%, 94%, 95%, 96%, 97%, 98%, or 99% identity to SEQ ID NO: 7. In some embodiments, the mRNA vaccines comprise a sequence that has at least 70%, 71%, 72%, 73%, 74%, 75%, 76%, 77%, 78%, 79%, 80%, 81%, 82%, 83%, 84%, 85%, 86%, 87%, 885, 89% 90%, 91%, 92%, 93%, 94%, 95%, 96%, 97%, 98%, or 99% identity to SEQ ID NO: 10. In some embodiments, the mRNA vaccines encode a polypeptide that is at least 70%, 71%, 72%, 73%, 74%, 75%, 76%, 77%, 78%, 79%, 80%, 81%, 82%, 83%, 84%, 85%, 86%, 87%, 885, 89%, 90%, 91%, 92%, 93%, 94%, 95%, 96%, 97%, 98%, 99% or identical to a sequence selected from SEQ ID NOs: 5, 8, and 12. In some embodiments, the mRNA vaccines encode a polypeptide that is at least 70%, 71%, 72%, 73%, 74%, 75%, 76%, 77%, 78%, 79%, 80%, 81%, 82%, 83%, 84%, 85%, 86%, 87%, 885, 89%, 90%, 91%, 92%, 93%, 94%, 95%, 96%, 97%, 98%, 99% or identical to SEQ ID NO: 5. In some embodiments, the mRNA vaccines encode a polypeptide that is at least 70%, 71%, 72%, 73%, 74%, 75%, 76%, 77%, 78%, 79%, 80%, 81%, 82%, 83%, 84%, 85%, 86%, 87%, 885, 89%, 90%, 91%, 92%, 93%, 94%, 95%, 96%, 97%, 98%, 99% or identical to SEQ ID NO: 8. In some embodiments, the mRNA vaccines encode a polypeptide that is at least 70%, 71%, 72%, 73%, 74%, 75%, 76%, 77%, 78%, 79%, 80%, 81%, 82%, 83%, 84%, 85%, 86%, 87%, 885, 89%, 90%, 91%, 92%, 93%, 94%, 95%, 96%, 97%, 98%, 99% or identical to SEQ ID NO: 12.

In some embodiments, the mRNAs are present in the compositions (e.g. mRNA vaccines) in an equal amount (e.g., a 1:1 weight/weight ratio or a 1:1 molar ratio), for example, a ratio of 1:1 (:1:1:1:1) of mRNA encoding distinct coronavirus antigens. As used herein, a “weight/weight ratio” or wt/wt ratio or wt:wt ratio refers to the ratio between the weights (masses) of the different components. A “molar ratio” refers to the ratio between different components (e.g., the number of mRNA encoding each antigen). In some embodiments, the ratio is 1:1, 1:2, 1:3, 1:4, 1:5, 1:6, 1:7, 1:8, 1:9, or 1:10.

In some embodiments, compositions (e.g. mRNA vaccines) comprise 25-75 μg of mRNA in total. In some embodiments, compositions (e.g. vaccines) comprise 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, or 75 μg mRNA in total. In some embodiments, compositions (e.g. mRNA vaccines) comprise 50 μg of mRNA in total.

In each embodiment or aspects, it is understood that vaccines include the mRNAs encapsulated within LNPs. While it is possible to encapsulate each unique mRNA in its own LNP, the mRNA vaccine technology enjoys the significant technological advantage of being able to encapsulate several mRNAs in a single LNP product. In other embodiments the vaccines are separate vaccines that are not co-formulated, but may be admixed separately before administration or simply administered separately.

### Nucleic Acids

In some aspects, compositions (e.g. mRNA vaccines) comprise a (at least one) messenger RNA (mRNA) having an open reading frame (ORF) encoding a coronavirus antigen. In some embodiments, the mRNA further comprises a 5′ UTR, 3′ UTR, a poly(A) tail and/or a 5′ cap analog.

It should also be understood that coronavirus vaccines may include any 5′ untranslated region (UTR) and/or any 3′ UTR. Exemplary UTR sequences include SEQ ID NOs: 2, 4, 50, and 51; however, other UTR sequences may be used or exchanged for any suitable UTR sequences. In some embodiments, a 5′ UTR comprises a sequence selected from SEQ ID NO: 13 (GGGAAAUA AGAGAGAAAAGAAGAGUAAGAAGAAAUAUAAGAGCCACC) and SEQ ID NO: 2 (GGGAAAUAAGAGAGAAAAGAAGAGUAAGAAGAAAUAUAAGACCCCGGCGCCGC CACC). In some embodiments, a 3′ UTR comprises a sequence selected from SEQ ID NO: 14 (UGAUAAUAGGCUGGAGCCUCGGUGGCCAUGCUU CUUGCCCCUUGGGCCUCCCCCCAGCCCCUCCUCCCCUUCCUGCACCCGUACCCCCG UGGUCUUUGAAUAAAGUCUGAGUGGGCGGC) and SEQ ID NO: 4 (UGAUAA UAGGCUGGAGCCUCGGUGGCCUAGCUUCUUGCCCCUUGGGCCUCCCCCCAGCCCC UCCUCCCCUUCCUGCACCCGUACCCCCGUGGUCUUUGAAUAAAGUCUGAGUGGGC GGC). UTRs may also be omitted from RNA polynucleotides.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNA sequences identified by a particular sequence identification number herein also disclose the corresponding mRNA sequence complementary to the DNA, where each “T” of the DNA sequence is substituted with “U.”

### Open Reading Frames (ORF)

An open reading frame (ORF) is a continuous stretch of DNA or RNA that (1) begins with a start codon (e.g., ATG or AUG, encoding methionine), and (2) ends with a stop codon (e.g., TAA, TAG or TGA, or UAA, UAG or UGA) or is immediately followed by a stop codon. A stop codon does not encode an amino acid, such that translation of an ORF terminates when a ribosome reaches the stop codon immediately following the last amino acid-encoding codon in the ORF. A stop codon that results in translation termination may be considered part of the ORF, in which case the ORF ends with the stop codon. Alternatively, the first stop codon immediately following the last amino acid-encoding codon of an ORF may considered part of the 3′ untranslated region (3′ UTR) of a DNA or RNA, rather than part of the ORF. Those skilled in the art will understand that an ORF sequence that ends in a codon encoding amino acid will be followed by one or more stop codons in a DNA or RNA. An ORF may be followed by multiple stop codons. Inclusion of multiple consecutive stop codons reduces the extent of continued translation that may occur if a stop codon is mutated to a codon encoding an amino acid (readthrough), as a second stop codon may terminate translation even if a first stop codon is mutated and encodes an amino acid, such that only one amino acid is added to the C-terminus of the translated protein. Where multiple stop codons are present at the end, or immediately following, an ORF, the multiple stop codons may comprise the same stop codon (e.g., UGAUGA). Multiple stop codons may comprise different stop codons in series (e.g., UGAUAAUAG). In addition to reducing the extent of readthrough if a first stop codon is mutated, the presence of multiple different stop codons reduces the extent of readthrough if the first stop codon fails to allow translation termination (e.g., if a suppressor tRNA with an anticodon complementary to the first stop codon is present in the cell).

An ORF typically encodes a protein. It will be understood that nucleotide sequences may further comprise additional elements, e.g., 5′ and/or 3′ UTRs, but that those elements, unlike the ORF, need not necessarily be present in an RNA (e.g., mRNA).

### Untranslated Regions (UTRs)

mRNAs may comprise one or more regions or parts which act or function as an untranslated region. A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the open reading frame (e.g., downstream from the last amino acid-encoding codon of an open reading frame, where the stop codon is considered part of the 3′ UTR, or downstream from the first stop codon signaling translation termination, where that stop codon is considered part of the open reading frame), and which does not encode a polypeptide. When RNA transcripts are being generated, the 5′ UTR may comprise a promoter sequence. Such promoter sequences are known in the art. It should be understood that such promoter sequences will not be present in vaccine compositions.

Where mRNAs encode a (at least one) protein, the mRNA may comprise a 5′ UTR and/or 3′ UTR. UTRs of an mRNA are transcribed but not translated. In mRNA, the 5′ UTR starts at the transcription start site and continues to the start codon but does not include the start codon; the 3′ UTR starts immediately following the open reading frame and continues until the transcriptional termination signal. Where an open reading frame ends with a codon encoding an amino acid, the 3′ UTR begins with a stop codon, such that no amino acids are added to a polypeptide beyond the last amino acid encoded by the open reading frame. A 3′ UTR may further comprise one or more stop codons. There is a growing body of evidence about the regulatory roles played by the UTRs in terms of stability of the nucleic acid molecule and translation. The regulatory features of a UTR can be incorporated into polynucleotides to, among other things, enhance the stability of the molecule. The specific features can also be incorporated to ensure controlled down-regulation of the transcript in case they are misdirected to undesired organs sites. A variety of 5′ UTR and 3′ UTR sequences are known.

In some embodiments, a 5′ UTR comprises a sequence selected from SEQ ID NO: 2 and SEQ ID NO: 13 or a sequence with at least 80%, 85%, 90%, 95%, 96%, 97%, 98%, 99% or 100% identity to SEQ ID NO: 2 and SEQ ID NO: 13, or a variant or a fragment thereof.

In some embodiments, the 5′ UTR comprises a sequence provided in Table 7 or a sequence with at least 80%, 85%, 90%, 95%, 96%, 97%, 98%, 99% or 100% identity to a 5′ UTR sequence provided in Table 7, or a variant or a fragment thereof.

In some embodiments, the 3′ UTR comprises a sequence provided in Table 8 or a sequence with at least 80%, 85%, 90%, 95%, 96%, 97%, 98%, 99% or 100% identity to a 3′ UTR sequence provided in Table 8, or a variant or a fragment thereof.

It should also be understood that mRNA may include any 5′ UTR and/or any 3′ UTR. UTRs may also be omitted from mRNA.

A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’.5′UTR also have been known to form secondary structures which are involved in elongation factor binding.

In some embodiments, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063; 9,012,219). CMV immediate-early 1 (IE1) gene (US 2014/0206753, WO 2013/185069), the sequence GGGAUCCUACC (SEQ ID NO: 17) (WO 2014/144196) may also be used. In another embodiment, 5′ UTR of a TOP gene is a 5′ UTR of a TOP gene lacking the 5′ TOP motif (the oligopyrimidine tract) (e.g., WO 2015/101414, WO 2015/101415, WO 2015/062738, WO 2015/024667, WO 2015/024667; 5′ UTR element derived from ribosomal protein Large 32 (L32) gene (WO 2015/101414, WO 2015/101415, WO 2015/062738), 5′ UTR element derived from the 5′UTR of an hydroxysteroid (17-β) dehydrogenase 4 gene (HSD17B4) (WO 2015/024667), or a 5′ UTR element derived from the 5′ UTR of ATP5A1 (WO 2015/024667) can be used. In some embodiments, an internal ribosome entry site (IRES) is used instead of a 5′ UTR.

A 3′ UTR does not encode a protein (is non-coding). Natural or wild-type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNAs. When engineering specific nucleic acids, one or more copies of an ARE can be introduced to make nucleic acids less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids and protein production can be assayed at various time points post-transfection. For example, cells can be transfected with different ARE-engineering molecules and by using an ELISA kit to the relevant protein and assaying protein produced at 6 hours, 12 hours, 1 day, 2 days, and 7 days post-transfection.

Those of ordinary skill in the art will understand that 5′ UTRs that are heterologous or synthetic may be used with any desired 3′ UTR sequence. For example, a heterologous or synthetic 5′ UTR may be used with a synthetic 3′ UTR or with a heterologous 3′ UTR.

Non-UTR sequences may also be used as regions or subregions within a nucleic acid. For example, introns or portions of introns sequences may be incorporated into regions of nucleic acids. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. A 5′ UTR may comprise a first polynucleotide fragment and a second polynucleotide fragment from the same and/or different genes such as the 5′ UTRs described in US2010/0293625 and WO2015/085318A2, each of which is herein incorporated by reference.

It should be understood that any UTR from any gene may be incorporated into the regions of a nucleic acid. Furthermore, multiple wild-type UTRs of any known gene may be utilized. Artificial UTRs which are not variants of wild-type regions may also be used. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type/native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US2010/0129877, which is incorporated herein by reference.

In some embodiments, UTRs may be patterned. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature, or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US 2009/0226470, herein incorporated by reference, and those known in the art.

### 5′ Caps

In some embodiments, an RNA (e.g., mRNA) comprises a 5′ terminal cap. 5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified RNA (e.g., mRNA) may be completed post-transcriptionally using, for example, a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs may be used.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

A nucleotide cap (e.g., a trinucleotide cap or tetranucleotide cap), in some embodiments, comprises a compound of formula (I)

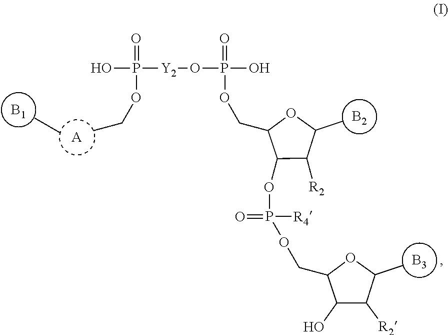

or a stereoisomer, tautomer or salt thereof, wherein

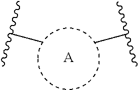

is

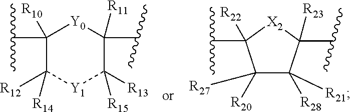

ring B₁ is a modified or unmodified Guanine;
ring B₂ and ring B3 each independently is a nucleobase or a modified nucleobase;
X₂ is O, S(O)ₚ, NR₂₄ or CR₂₅R₂₆ in which p is 0, 1, or 2;
Y₀ is O or CR₆R₇;
Y1 is O, S(O)ₙ, CR₆R₇, or NR₈, in which n is 0, 1, or 2;
each --- is a single bond or absent, wherein when each -- is a single bond, Yi is O, S(O)ₙ, CR₆R₇, or NR₈; and when each -- is absent, Y₁ is void;
Y₂ is (OP(O)R₄)ₘ in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;
each R₂ and R_{2′} independently is halo, LNA, or OR₃;
each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
each R₄ and R_{4′} independently is H, halo, C₁-C₆ alkyl, OH, SH, SeH, or BH₃⁻;
each of R₆, R₇, and R₄, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;
each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered
heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered
heterocycloalkyl, and 5- or 6-membered heteroaryl; or alternatively R₁₂ together with R₁₄ is oxo, or R₁₃ together with R₁₅ is oxo,
each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rs₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
each of R₂₄, R₂₅, and R₂₆ independently is H or C₁-C₆ alkyl;
each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;
R₃₀ is C₁-C₆ alkylene optionally substituted with one or more of halo, OH and C₁-C₆ alkoxyl;
each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;
each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;
R₄₄ is H, C₁-C₆ alkyl, or an amine protecting group;
each of R₄₅ and R₄₆ independently is H, OP(O)R₄₇R₄₈, or C₁-C₆ alkyl optionally substituted with one or more OP(O)R₄₇R₄₈, and
each of R₄₇ and R₄₈, independently is H, halo, C₁-C₆ alkyl, OH, SH, SeH, or BH₃⁻.

It should be understood that a cap analog may include any of the cap analogs described in international publication WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a tetranucleotide cap comprises the following structure:

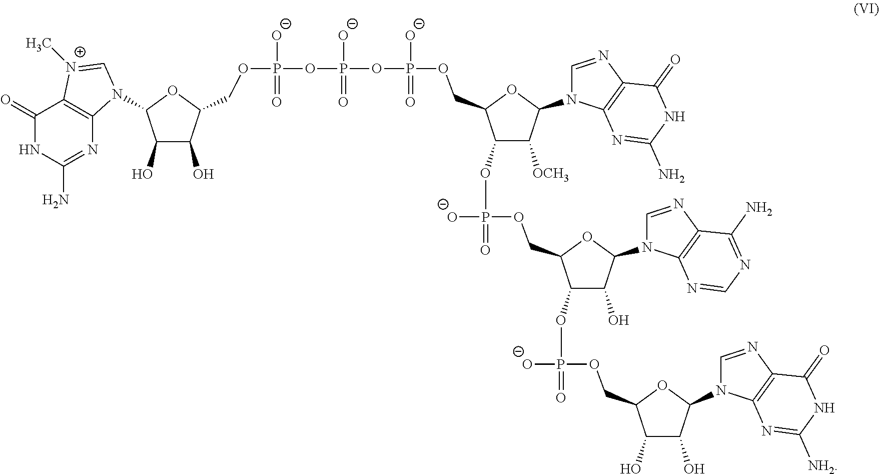 [SMILES: CO[C@@H]1[C@H](OP(=O)([O-])OC[C@H]2O[C@@H](n3cnc4c(N)ncnc43)[C@H](O)[C@@H]2OP(=O)([O-])OC[C@H]2O[C@@H](n3cnc4c(=O)[nH]c(N)nc43)[C@H](O)[C@@H]2O)[C@@H](COP(=O)([O-])OP(=O)([O-])OP(=O)([O-])OC[C@H]2O[C@@H](n3c[n+](C)c4c(=O)[nH]c(N)nc43)[C@H](O)[C@@H]2O)O[C@H]1n1cnc2c(=O)[nH]c(N)nc21]

In other embodiments, a tetranucleotide cap comprises the following structure:

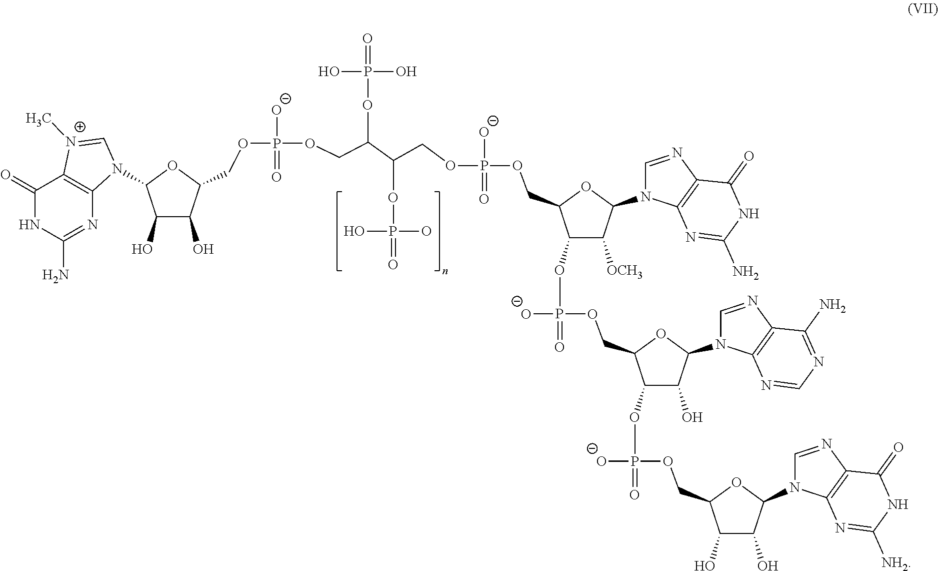

In yet other embodiments, a tetranucleotide cap comprises the following structure:

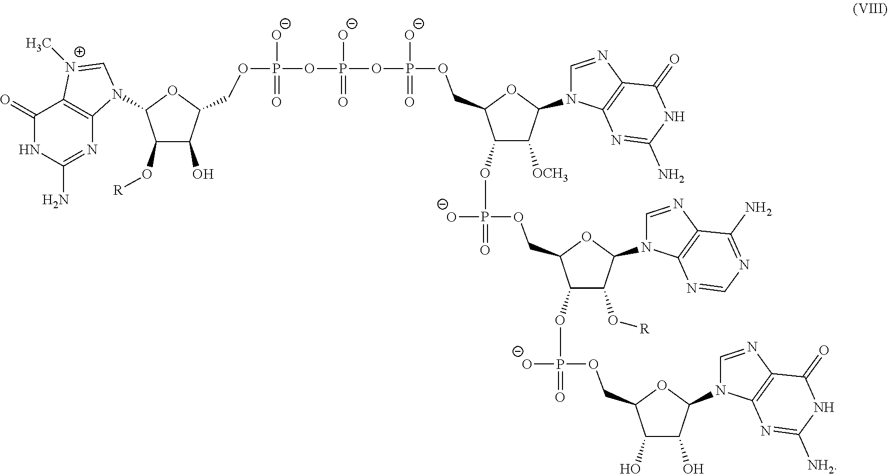

In yet other embodiments, a tetranucleotide cap comprises the following structure:

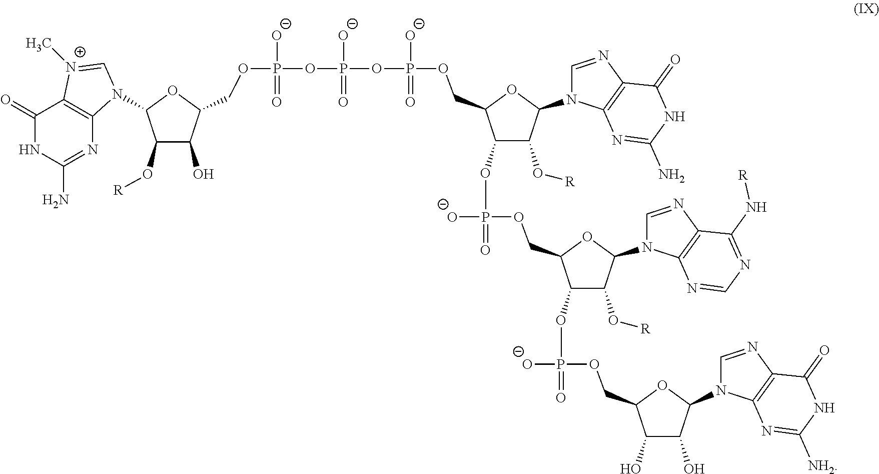

In some embodiments, R is an alkyl (e.g., C₁-C₆ alkyl). In some embodiments, R is a methyl group (e.g., C₁ alkyl). In some embodiments, R is an ethyl group (e.g., C₂ alkyl). In some embodiments, R is a hydrogen.

In some embodiments, a tetranucleotide cap comprises GGAG.

In some embodiments, a tetranucleotide cap comprises any one of the following structures:

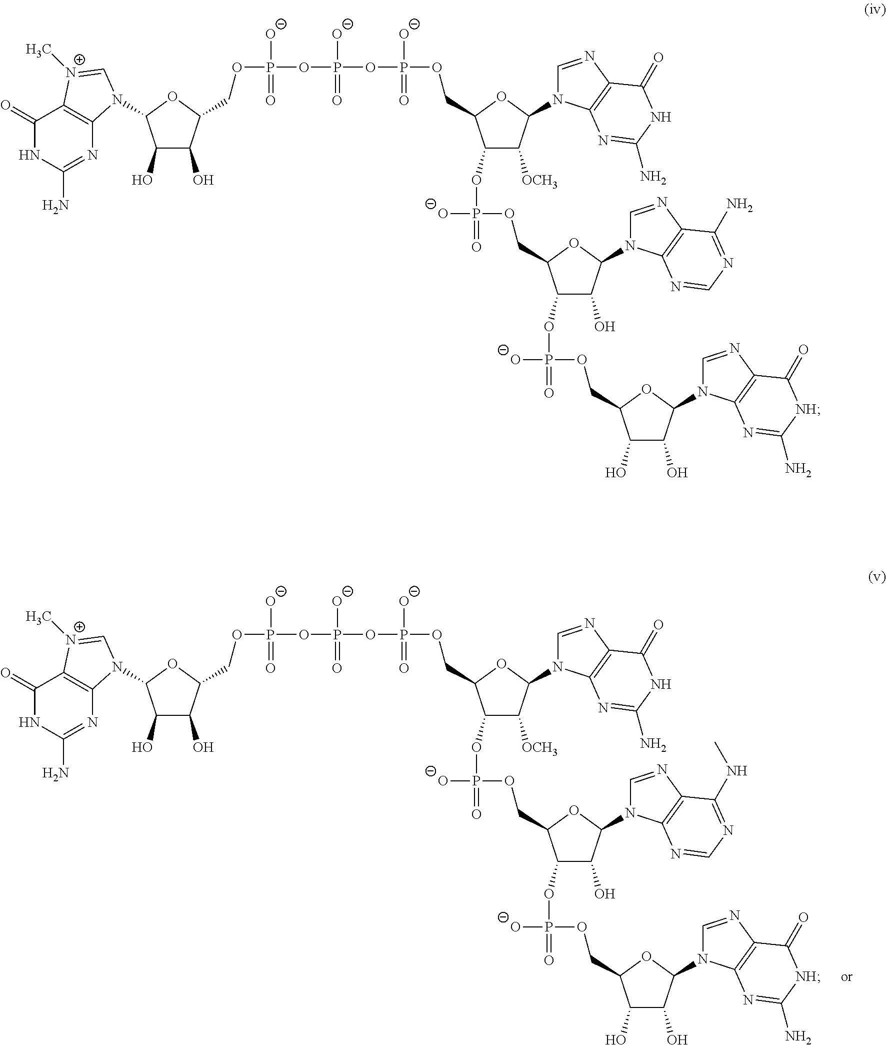

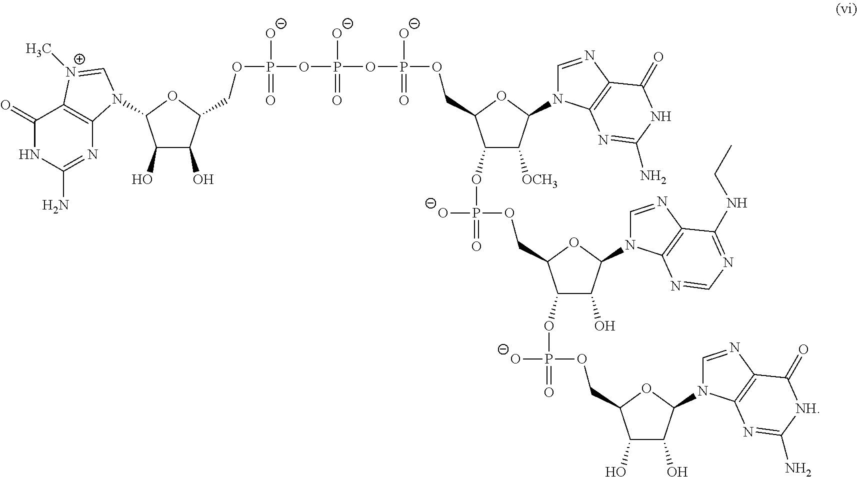 [SMILES: CCNc1ncnc2c1ncn2[C@@H]1O[C@H](COP(=O)([O-])O[C@H]2[C@@H](OC)[C@H](n3cnc4c(=O)[nH]c(N)nc43)O[C@@H]2COP(=O)([O-])OP(=O)([O-])OP(=O)([O-])OC[C@H]2O[C@@H](n3c[n+](C)c4c(=O)[nH]c(N)nc43)[C@H](O)[C@@H]2O)[C@@H](OP(=O)([O-])OC[C@H]2O[C@@H](n3cnc4c(=O)[nH]c(N)nc43)[C@H](O)[C@@H]2O)[C@H]1O]

### Variants

In some embodiments, compositions (e.g. mRNA vaccines) include RNA that encodes a SARS-CoV-2 antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Examples of SARS-CoV-2 antigen variants are provided in Table 1. Ordinarily, variants possess at least 50% identity to a wild-type, native or reference sequence. In some embodiments, variants share at least 90% identity with a wild-type, native, or reference sequence. In some embodiments, nucleic acid vaccines encode SARS-CoV-2 variants comprising 1, 2, 3, 4, or more mutations relative to a reference sequence. In some embodiments, nucleic acid vaccines encode SARS-CoV-2 variants comprising less than 20, 18, 15, 12, or 10 mutations relative to a reference sequence. In some embodiments, nucleic acid vaccines encode SARS-CoV-2 variants having 1-50 1-40, 1-30, 1-25, 1-20, 1-15, 1-10, 5-50, 5-40, 5-30, 5-25, 5-20, 5-15, 5-10, 10-50, 10-40, 10-30, 10-25, 10-20, 10-15, 20-50, 20-40, 20-30, 20-25, 25-50, 25-40, 25-30, 30-50, 30-40, 40-50 mutations (e.g., substitutions). As used herein, “mutation” refers to an amino acid substitution, insertion, or deletion. A reference sequence refers to a naturally-occurring strain, for example, a naturally-occurring circulating strain of SARS-CoV-2.

Variant antigens/polypeptides encoded by nucleic acids may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein, or comprises a nucleotide sequence at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 90%, 91%, 92%, 93%, 94%, 95%, 96%, 97%, 98%, 99% but less than 100% sequence identity to that particular reference polynucleotide or polypeptide as determined by sequence alignment programs and parameters, including those known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, in some aspects, polynucleotides encode peptides or polypeptides containing substitutions, insertions and/or additions, deletions and covalent modifications with respect to reference sequences, in particular polypeptide (e.g., antigen) sequences. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of coronavirus antigens of interest. Non-limiting examples of such antigens include any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39,40, 41,42, 43,44, 45,46,47, 48,49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid.

### Scaffold Moieties

In some embodiments, mRNA vaccines encode fusion proteins that comprise coronavirus antigens linked to one another or scaffold moieties. In some embodiments, such scaffold moieties impart desired properties to an antigen encoded by a nucleic acid. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments, the coronavirus antigen is fused to HBsAG or HBcAG to facilitate self-assembly of nanoparticles displaying the coronavirus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, an RNA encodes a coronavirus antigen fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, mRNAs encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS (SEQ ID NO: 57) linker. In some embodiments, the fusion protein contains three domains with intervening linkers, having the structure: domain-linker-domain-linker-domain.

In some embodiments, peptides comprise cleavable linkers known in the art. Exemplary such linkers include: F2A linkers, T2A linkers, P2A linkers, E2A linkers (See, e.g., WO2017/127750), and other art-recognized linkers. The skilled artisan will appreciate that other polycistronic constructs (mRNA encoding more than one antigen/polypeptide separately within the same molecule) may be suitable for vaccine formulations.

### Sequence Modification

In some embodiments, an open reading frame encoding a protein is codon optimized. Codon optimization methods are known in the art. An open reading frame of any one or more of the sequences provided herein may be codon optimized. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame sequence is optimized using optimization algorithms.

In some embodiments, a codon optimized sequence shares less than 95% sequence identity to a naturally-occurring or wild-type sequence open reading frame (e.g., a naturally-occurring or wild-type RNA (e.g., mRNA) sequence encoding a SARS-CoV-2 protein antigen). In some embodiments, a codon optimized sequence shares less than 90% sequence identity to a naturally-occurring or wild-type sequence (e.g., a naturally-occurring or wild-type RNA (e.g., mRNA) sequence encoding a SARS-CoV-2 protein). In some embodiments, a codon optimized sequence shares less than 85% sequence identity to a naturally-occurring or wild-type sequence (e.g., a naturally-occurring or wild-type RNA (e.g., mRNA) sequence encoding a SARS-CoV-2 protein). In some embodiments, a codon optimized sequence shares less than 80% sequence identity to a naturally-occurring or wild-type sequence (e.g., a naturally-occurring or wild-type RNA (e.g., mRNA) sequence encoding a SARS-CoV-2 protein). In some embodiments, a codon optimized sequence shares less than 75% sequence identity to a naturally-occurring or wild-type sequence (e.g., a naturally-occurring or wild-type RNA (e.g., mRNA) sequence encoding a SARS-CoV-2 protein).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type RNA (e.g., mRNA) sequence encoding a SARS-CoV-2 protein). In some embodiments, a codon optimized sequence shares between 65% and 75% or about 80% sequence identity to a naturally-occurring or wild-type sequence (e.g., a naturally-occurring or wild-type RNA (e.g., mRNA) sequence encoding a SARS-CoV-2 protein).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a SARS-CoV-2 protein encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA (e.g., mRNA) may be one in which the levels of G/C are enhanced. The G/C-content of nucleic acid molecules (e.g., mRNA) may influence the stability of the RNA. RNA (e.g., mRNA) having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an RNA (e.g., mRNA) stabilized by sequence modifications in the translated region. Due to the degeneracy of the genetic code, the modifications work by substituting existing codons for those that promote greater RNA stability without changing the resulting amino acid. The approach is limited to coding regions of the RNA (e.g., mRNA).

Some embodiments of mRNAs comprise a sequence with a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%. In some embodiments, the nucleic acid sequence of the full-length mRNA comprises a % G/C content of 30%-80%, 40%-70%, 50%-60%, 35%-50%, 50%-65%, 65%-70%, 40%-45%, 45%-50%, 50%-55%, 55%-70%, 70%-75%, or 75%-80%.

In some embodiments, the mRNA comprises an ORF with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, the mRNA comprises 5′ UTR with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, the mRNA comprises 3′ UTR with a % G/C content from about 30% to about 80%, about 35% to about 70%, about 40% to about 60%, about 45% to about 55%, about 40% to about 70%, about 50% to about 60%, about 35% to about 50%, about 50% to about 50% to about 65%, about 65% to about 70%, about 40% to about 45%, about 45% to about 50%, about 50% to about 55%, about 55% to about 70%, about 70% to about 75%, or about 75% to about 80%. In some embodiments, a modified mRNA comprises a higher % G/C content than a wild-type mRNA sequence. In some embodiments, the % G/C content of the modified mRNA sequence is 2% or more, 3% or more, 4% or more, 5% or more, 6% or more, 7% or more, 8% or more, 9% or more, 10% or more, 12% or more, 15% or more, or 20% or more than the % G/C content of the wild-type RNA sequence. In some embodiments, the % G/C content of the modified ORF sequence is 2% or more, 3% or more, 4% or more, 5% or more, 6% or more, 7% or more, 8% or more, 9% or more, 10% or more, 12% or more, 15% or more, or 20% or more than the % G/C content of the wild-type ORF sequence. In some embodiments, the % G/C content of the modified 5′ UTR sequence is 2% or more, 3% or more, 4% or more, 5% or more, 6% or more, 7% or more, 8% or more, 9% or more, 10% or more, 12% or more, 15% or more, or 20% or more than the % G/C content of the wild-type 3′ UTR sequence.

### Chemical Modifications

In some aspects, compositions comprise an RNA having an open reading frame encoding a protein, wherein the nucleic acid comprises nucleotides and/or nucleosides that can be standard (unmodified) or modified as is known in the art. In some embodiments, nucleotides and nucleosides comprise modified nucleotides or nucleosides. Such modified nucleotides and nucleosides can be naturally-occurring modified nucleotides and nucleosides or non-naturally-occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide is one as is generally known or recognized in the art. Non-limiting examples of such naturally-occurring modified nucleotides and nucleotides can be found, inter alia, in the widely recognized MODOMICS database.

In some embodiments, a non-naturally-occurring modified nucleotide or nucleoside of is one as is generally known or recognized in the art. Non-limiting examples of such non-naturally-occurring modified nucleotides and nucleosides can be found, inter alia, in international publication numbers WO2013052523A1; WO2014093924A1; WO2015051173A2; WO2015051169A2; WO2015089511A2; or WO2017153936A1, each of which is herein incorporated by reference in its entirety.

Hence, nucleic acids (e.g., DNA and RNA, such as mRNA) can comprise standard nucleotides and nucleosides, naturally-occurring nucleotides and nucleosides, non-naturally-occurring nucleotides and nucleosides, or any combination thereof.

Nucleic acids (e.g., DNA and RNA, such as mRNA), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA (e.g., mRNA) introduced to a cell or organism, exhibits reduced degradation in the cell or organism, respectively, relative to an unmodified nucleic acid comprising standard nucleotides and nucleosides.

In some embodiments, a modified RNA (e.g., mRNA) introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA, such as mRNA), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

Nucleic acids (e.g., RNA, such as mRNA) may comprise modified nucleosides and nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA comprises one or more chemical modifications. In some embodiments, a mRNA comprises 2, 3, 4, 5, 6, 7, 8, 9, 10, or more chemical modifications. In some embodiments, a mRNA is fully chemically modified.

In some embodiments, a mRNA comprises 1-methyl-pseudouridine (m1ψ) substitutions at one or more or all uridine positions of the nucleic acid.

In some embodiments, a mRNA comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA comprises pseudouridine (ψ) substitutions at one or more or all uridine positions of the nucleic acid.

In some embodiments, a mRNA comprises pseudouridine (ψ) substitutions at one or more or all uridine positions of the nucleic acid and 5-methyl cytidine substitutions at one or more or all cytidine positions of the nucleic acid.

In some embodiments, a mRNA comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

In some aspects, nucleic acids are partially or fully modified along the entire length of the molecule. For example, one or more or all or a given type of nucleotide (e.g., purine or pyrimidine, or any one or more or all of A, G, U, C) may be uniformly modified in a nucleic acid, or in a predetermined sequence region thereof (e.g., in the mRNA including or excluding the poly(A) tail). In some embodiments, all nucleotides X in a nucleic acid (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

Modified nucleotides may include modified nucleobases. For example, an RNA transcript (e.g., mRNA transcript) may include a modified uracil nucleobase selected from pseudouracil (ψ), N1-methylpseudouracil (m1ψ), 1-methylpseudouracil, 2-thiouracil, 4′-thiouracil, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pseudouracil, 5-aza-uracil, dihydropseudouracil, 5-methyluracil, 5-methoxyuracil (mo5U) and 2′-O-methyluracil. In some embodiments, an RNA transcript (e.g., mRNA transcript) includes a modified guanine nucleobase selected from digoxigeninated guanine, 6-thioguanine, 7-deazaguanine, 7-deaza-7-propargylaminoguanine, 8-oxoguanine, araguanine, biotin-16-7-deaza-7-propargylaminoguanine, isoguanine, N2-methylguanine, 06-methylguanine, thioguanine, and 2,6-daminoguanine. In some embodiments, an RNA transcript may include a modified cytosine nucleobase selected from digoxigeninated cytosine, 2-thiocytosine, 5-aminoallylcytosine, 5-bromocytosine, 5-carboxycytosine, 5-formylcytosine, 5-hydroxycytosine, 5-hydroxymethylcytosine, 5-methoxycytosine, 5-methylcytosine, 5-propargylaminocytosine, 5-propynylcytosine, 6-azacytosine, aracytosine, cyanine 3-5-propargylaminocytosine, cyanine 3-aminoallylcytosine, cyanine 5-6-propargylaminocytosine, cyanine 5-aminoallylcytosine, desthiobiotin-6-aminoallylcytosine, N4-biotin-OBEA-cytosine, N4-methylcytosine, pseudoisocytosine, and thienocytosine. In some embodiments, an RNA transcript (e.g., mRNA transcript) includes a modified adenine nucleobase selected from digoxigeninated adenine, N6-methyladenine, 7-deazaadenine, 7-deaza-7-propargylaminoadenine, 8-azaadenine, 8-azidoadenine, 8-chloroadenine, 8-oxoadenine, araadenine, N1-methyladenine, N6-methyladenine, 3-deazaadenine, 2,6-diaminoadenine, 2-methyl-thio-N6-isopentenyladenine (1s2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, an RNA transcript (e.g., mRNA transcript) includes a combination of at least two (e.g., 2, 3, 4 or more) of the foregoing modified nucleobases.

Modified nucleotides may include modified sugars. For example, an RNA transcript (e.g., mRNA transcript) may include a modified sugar selected from 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, an RNA transcript (e.g., mRNA transcript) includes a combination of at least two (e.g., 2, 3, 4 or more) of the foregoing modified sugars.

Modified nucleotides may include modified phosphates. A modified phosphate group is a phosphate group that differs from the canonical structure of phosphate. An example of a canonical structure of a phosphate is shown below:

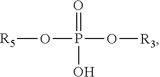

where R₅ and R₃ are atoms or molecules to which the canonical phosphate is bonded. For example, for a phosphate in a nucleic acid sequence, R₅ may refer to the upstream nucleotide of the nucleic acid, and R₃ may refer to the downstream nucleotide of the nucleic acid. The canonical structure of phosphate also refers to structures in which one or more hydroxyl groups of the phosphate are deprotonated, or in which an oxygen atom of the phosphate is bonded to an adjacent nucleotide in a nucleic acid sequence. In some embodiments, an RNA transcript (e.g., mRNA transcript) may include a modified phosphate selected from phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, an RNA transcript (e.g., mRNA transcript) includes a combination of at least two (e.g., 2, 3, 4 or more) of the foregoing modified phosphates.

In some embodiments, an mRNA includes N1-methylpseudouridine. In some embodiments, at least 70%, at least 80%, at least 85%, at least 90%, at least 95%, at least 97%, at least 98%, or at least 99% of uracil nucleotides in an mRNA comprise N1-methylpseudouridine. In some embodiments, each uracil nucleotide of an mRNA transcript comprises N1-methylpseudouridine. In some embodiments, an mRNA includes 5-methylcytidine. In some embodiments, at least 70%, at least 80%, at least 85%, at least 90%, at least 95%, at least 97%, at least 98%, or at least 99% of cytosine nucleotides in an mRNA comprise 5-methylcytidine. In some embodiments, each cytosine nucleotide of an mRNA transcript comprises 5-methylcytidine. In some embodiments, an mRNA includes 5-methyluridine. In some embodiments, at least 70%, at least 80%, at least 85%, at least 90%, at least 95%, at least 97%, at least 98%, or at least 99% of uracil nucleotides in an mRNA comprise 5-methyluridine. In some embodiments, each uracil nucleotide of an mRNA transcript comprises 5-methyluridine. In some embodiments, an mRNA includes 5-methylcytidine and 5-methyluridine. In some embodiments, at least 70%, at least 80%, at least 85%, at least 90%, at least 95%, at least 97%, at least 98%, or at least 99% of uracil nucleotides in an mRNA comprise 5-methyluridine and at least 70%, at least 80%, at least 85%, at least 90%, at least 95%, at least 97%, at least 98%, or at least 99% of cytosine nucleotides in an mRNA comprise 5-methylcytidine. In some embodiments, each cytosine nucleotide of an mRNA transcript comprises 5-methylcytidine and each uracil nucleotide of an mRNA transcript comprises 5-methyluridine.

### Unmodified Nucleotides

In some embodiments, an mRNA is not chemically modified and comprises the standard ribonucleotides consisting of adenosine, guanosine, cytosine and uridine. In some embodiments, nucleotides and nucleosides comprise standard nucleoside residues such as those present in transcribed RNA (e.g. A, G, C, or U). In some embodiments, nucleotides and nucleosides comprise standard deoxyribonucleosides such as those present in DNA (e.g. dA, dG, dC, or dT).

### Identification and Ratio Determination (IDR) Sequences

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding a therapeutic and/or antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### In Vitro Transcription of RNA

cDNA encoding the polynucleotides may be transcribed using an in vitro transcription (IVT) system. In vitro transcription of RNA is known in the art and is described in International Publication WO 2014/152027, which is incorporated by reference herein in its entirety. In some embodiments, RNA is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to influenza virus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

An in vitro transcription system typically comprises a transcription buffer, nucleotide triphosphates (NTPs), an RNase inhibitor and a polymerase.

The NTPs may be manufactured in house, may be selected from a supplier, or may be synthesized. The NTPs may be selected from, but are not limited to, natural and unnatural (modified)NTPs.

Any number of RNA polymerases or variants may be used. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

Co-transcriptional capping methods for ribonucleic acid (RNA) synthesis, using any RNA polymerase variant, may also be used to generate RNA. That is, RNA may be produced in a “one-pot” reaction, without the need for a separate capping reaction. Thus, the methods, in some embodiments, comprise reacting a polynucleotide template with a RNA polymerase variant, nucleoside triphosphates, and a cap analog under in vitro transcription reaction conditions to produce RNA transcript.

### Chemical Synthesis

Solid-phase chemical synthesis. Nucleic acids may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids by the sequential addition of monomer building blocks may be carried out in a liquid phase.

Combination of Synthetic Methods. The synthetic methods discussed above each have their own advantages and limitations. Attempts have been conducted to combine these methods to overcome the limitations, and such combinations of methods may be used to generate nucleic acids. For example, the use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of nucleic acids may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, nucleic acids may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because nucleic acids, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

### Formula (AI)

In some embodiments, the ionizable amino lipid of a lipid nanoparticle is a compound of Formula (AI):

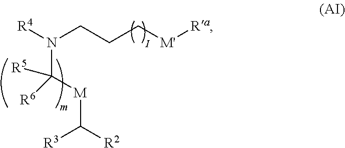

or its N-oxide, or a salt or isomer thereof,
wherein R′^{a} is R′^{branched}; wherein
R′^{branched} is:

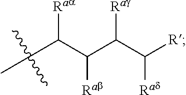

wherein

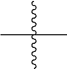

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

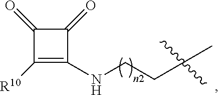

wherein

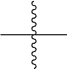

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

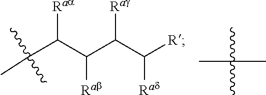

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

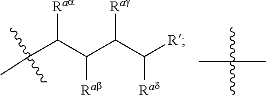

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (AI), R′^{a} is R′^{branched}; R′^{branched} is

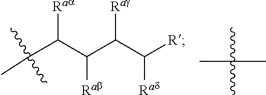

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

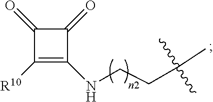

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (AI), R′^{a} is R′^{branched}; R′^{branched} is

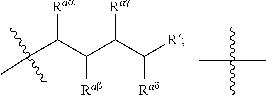

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (AI) is selected from:

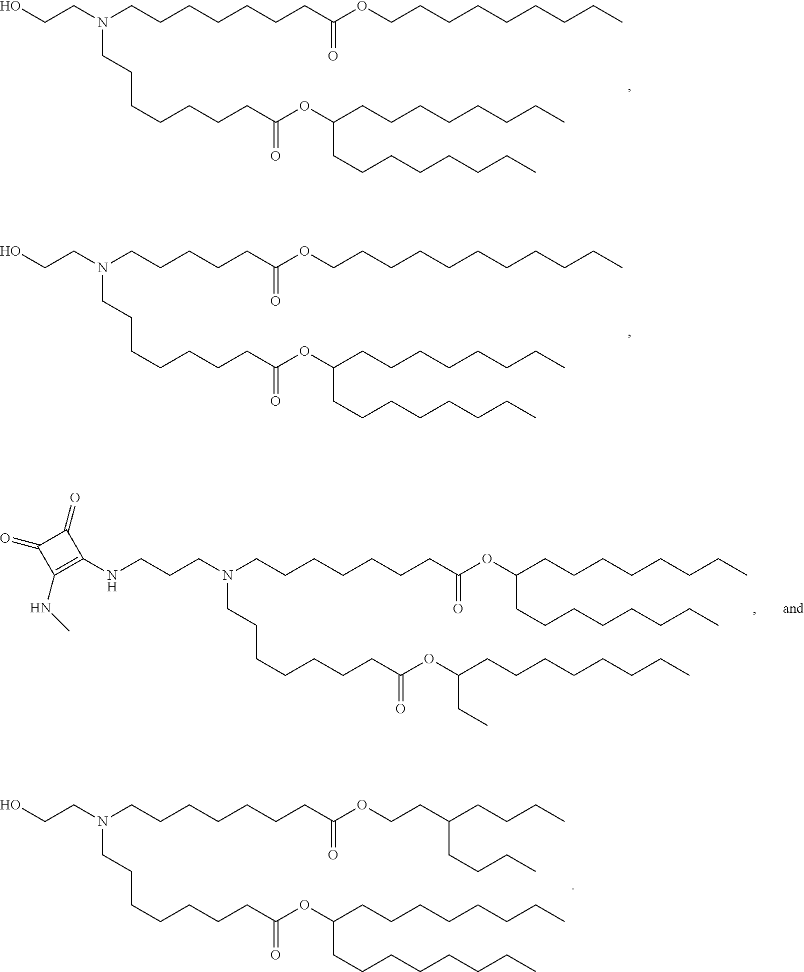 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

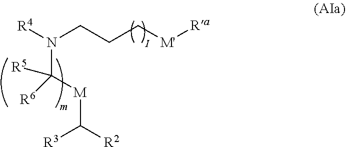

or its N-oxide, or a salt or isomer thereof,
wherein R′^{a} is R′^{branched}; wherein
R′^{branched} is:

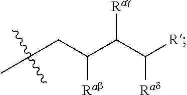

wherein

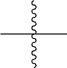

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

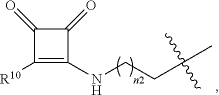

wherein

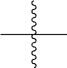

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

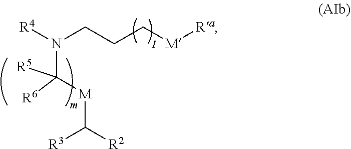

or its N-oxide, or a salt or isomer thereof,
wherein R′^{a} is R′^{branched}; wherein
R′^{branched} is:

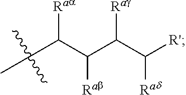

wherein

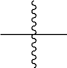

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

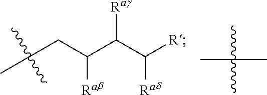

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (AI) or (AIb), R′^{a} is R′^{branched}; R′^{branched} is

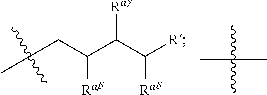

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (AI) or (AIb), R′^{a} is R′^{branched}; R′^{branched} is

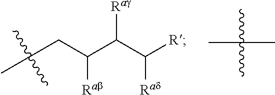

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the ionizable amino lipid of Formula (AI) is a compound of Formula (AIc):

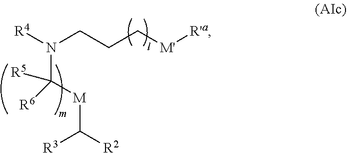

or its N-oxide, or a salt or isomer thereof,
wherein R′^{a} is R′^{branched}; wherein
R′^{branched} is:

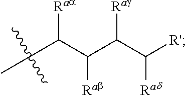

wherein

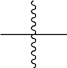

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

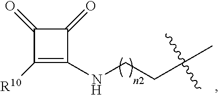

wherein

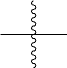

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

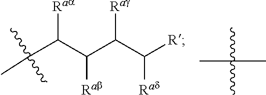

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

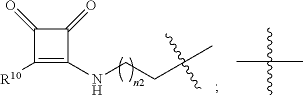

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (AIc) is:

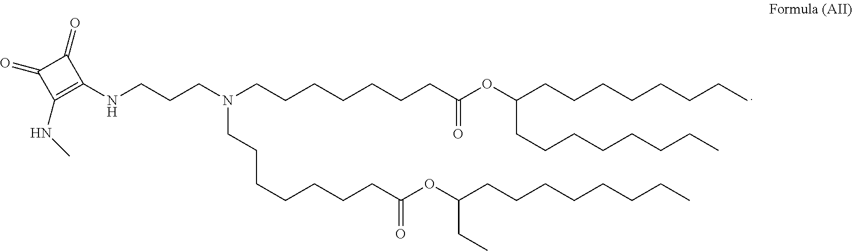 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

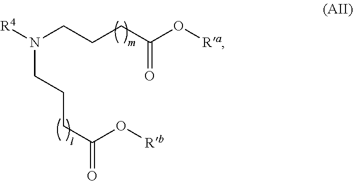

or its N-oxide, or a salt or isomer thereof,
wherein R′^{a} is R′^{branched} or R′^{cyclic}; wherein
R′^{branched} is:

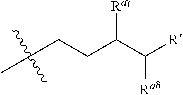

and R′^{cyclic} is:

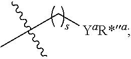

and
R′^{b} is:

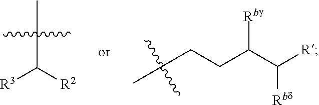

wherein

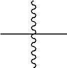

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

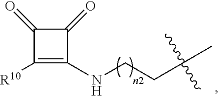

wherein

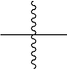

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

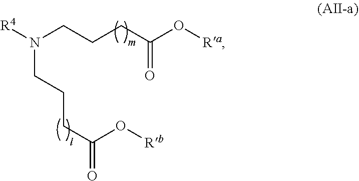

or its N-oxide, or a salt or isomer thereof,
wherein R′^{a} is R′^{branched} or R′^{cyclic}; wherein
R′^{branched} is:

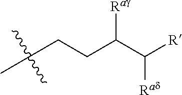

and R′^{b} is:

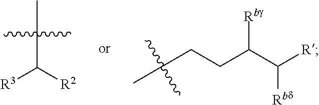

wherein

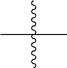

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

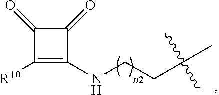

wherein

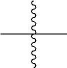

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

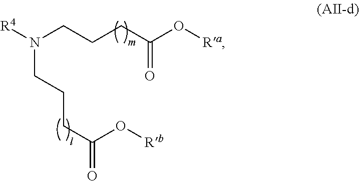

or its N-oxide, or a salt or isomer thereof,
wherein R′^{a} is R′^{branched} or R′^{cyclic}; wherein
R′^{branched} is:

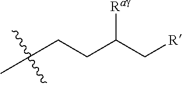

and R′^{b} is:

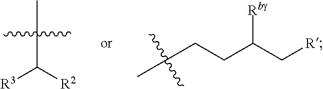

wherein

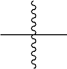

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

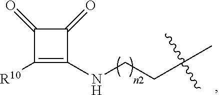

wherein

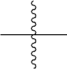

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

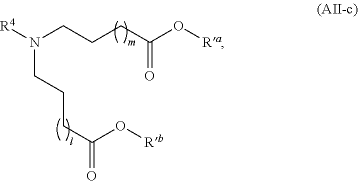

or its N-oxide, or a salt or isomer thereof,
wherein R′^{a} is R′^{branched} or R′^{cyclic}; wherein
R′^{branched} is:

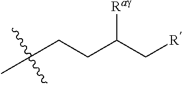

and R′^{b} is:

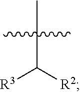

wherein

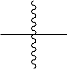

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

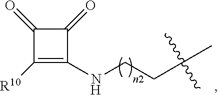

wherein

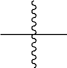

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

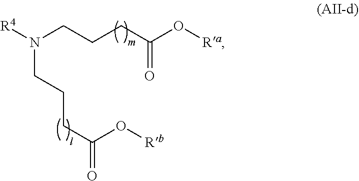

or its N-oxide, or a salt or isomer thereof,
wherein R′^{a} is R′^{branched} or R′^{cyclic}; wherein
R′^{branched} is:

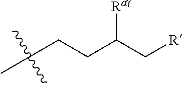

and R′^{b} is:

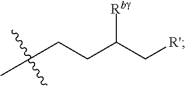

wherein

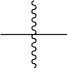

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

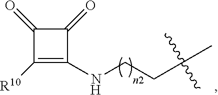

wherein

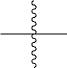

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

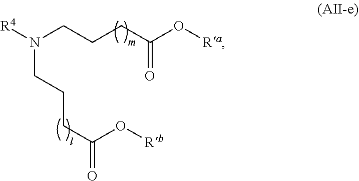

or its N-oxide, or a salt or isomer thereof,
wherein R′^{a} is R′^{branched} or R′^{cyclic}; wherein
R′^{branched} is:

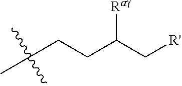

and R′^{b} is:

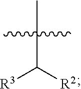

wherein

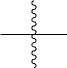

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and 1 are each independently selected from 4, 5, and 6. In some embodiments of the compound of Formula (AII), (AII-a), (AII-b), (AII-c), (AII-d), or (AII-e), m and 1 are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

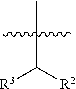

and R² and R³ are each independently a C₁₋₁₄ alkyl. In some embodiments of the compound of Formula (AII), (AII-a), (AII-b), (AII-c), (AII-d), or (AII-e), R′^{b} is:

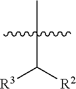

R² and R³ are each independently a C₆₋₁₀ alkyl. In some embodiments of the compound of Formula (AII), (AII-a), (AII-b), (AII-c), (AII-d), or (AII-e), R′^{b} is:

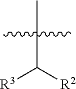

and R² and R³ are each a C₈ alkyl.

In some embodiments of the compound of Formula (AII), (AII-a), (AII-b), (AII-c), (AII-d), or (AII-e), R′^{branched} is:

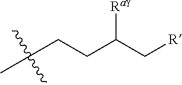

### and R′^{b} is:

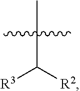

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

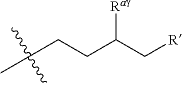

### and R′^{b} is:

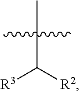

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

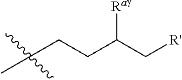

### and R′^{b} is:

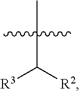

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

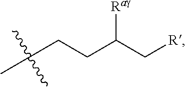

### R′^{b} is:

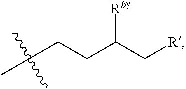

and R^{aγ} and R^{bγ} are each a C₁₋₁₂ alkyl. In some embodiments of the compound of Formula (AI), (AII-a), (AII-b), (AII-c), (AII-d), or (AII-e), R′^{branched} is:

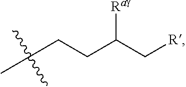

### R′^{b} is:

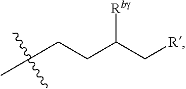

and R^{aγ} and R^{bγ} are each a C₂₋₆ alkyl.

In some embodiments of the compound of Formula (AII), (AII-a), (AII-b), (AII-c), (All-d), or (AII-e), m and 1 are each independently selected from 4, 5, and 6 and each R′ independently is a C₁₋₁₂ alkyl. In some embodiments of the compound of Formula (AI), (AII-a), (AII-b), (AII-c), (AII-d), or (AII-e), m and 1 are each 5 and each R′ independently is a C₂₋₅ alkyl.

In some embodiments of the compound of (AII), (AII-a), (AII-b), (AII-c), (AII-d), or (AII-e), R′^{branched} is:

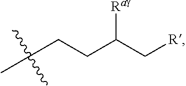

### R′^{b} is:

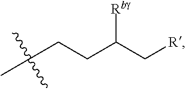

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

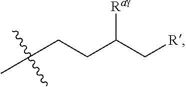

### R′^{b} is:

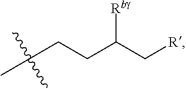

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

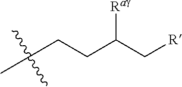

### and R′^{b} is:

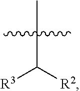

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

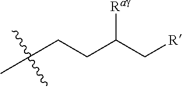

### and R′^{b} is

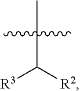

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

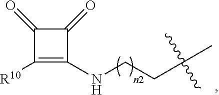

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

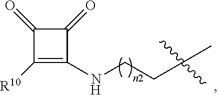

wherein R¹⁰ is NH(CH₃) and n2 is 2.

In some embodiments of the compound of Formula (AII), (AII-a), (AI-b), (AII-c), (AII-d), or (AII-e), R′^{branched} is:

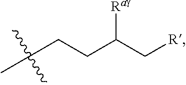

### R′^{b} is:

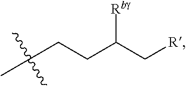

m and l are each independently selected from 4, 5, and 6, each R′ independently is a C₁₋₁₂ alkyl, R^{aγ} and R^{bγ} are each a C₁₋₁₂ alkyl, and R⁴ is

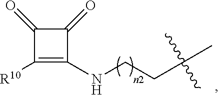

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

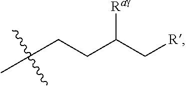

### R′^{b} is:

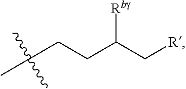

m and l are each 5, each R′ independently is a C₂₋₅ alkyl, R^{aγ} and R^{bγ} are each a C₂₋₆ alkyl, and R⁴ is

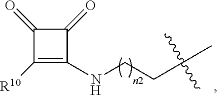

wherein R¹⁰ is NH(CH₃) and n2 is 2.

In some embodiments of the compound of Formula (AII), (AII-a), (AII-b), (AII-c), (AII-d), or (AII-e), R′^{branched} is:

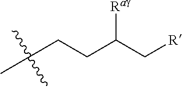

### and R′^{b} is:

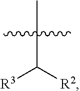

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

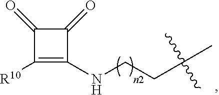

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is

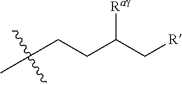

### and R′^{b} is:

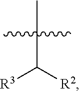

m and l are each 5, R′ is a C₂₋₅ alkyl, R^{aγ} is a C₂₋₆ alkyl, R² and R³ are each a C₈ alkyl, and R⁴ is

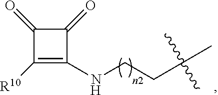

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

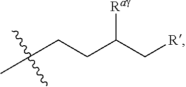

### R′^{b} is:

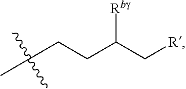

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AI), (AII-a), (AII-b), (AII-c), (AII-d), or (AII-e), R′^{branched} is:

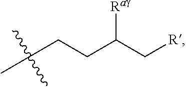

### R′^{b} is:

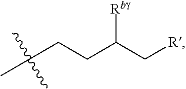

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

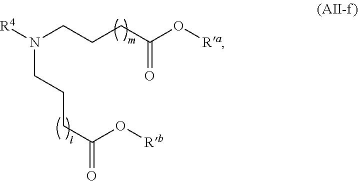

or its N-oxide, or a salt or isomer thereof,
wherein R′^{a} is R′^{branched} or R′^{cyclic}; wherein
R′^{branched} is:

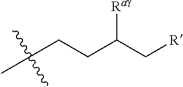

and R′^{b} is:

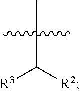

wherein

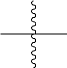

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and 1 are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

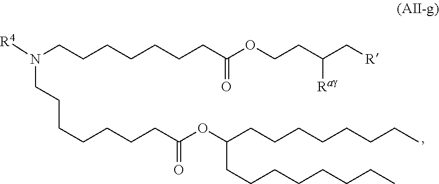

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

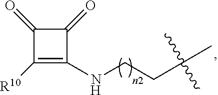

wherein

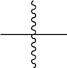

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

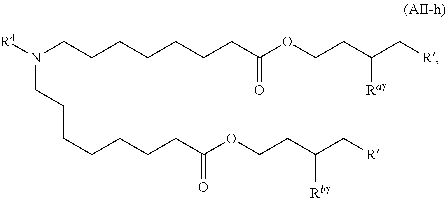

or its N-oxide, or a salt or isomer thereof; wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

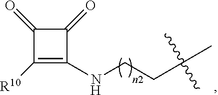

wherein

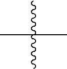

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

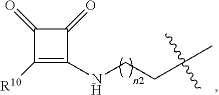

wherein
R¹⁰ is NH(CH₃) and n2 is 2.

In some embodiments of the compound of Formula (AII-g) or (AII-h), R⁴ is —(CH₂)₂OH.

### Formula (AIII)

In some embodiments, the ionizable amino lipids of a lipid nanoparticle may be one or more of compounds of Formula (AIII):

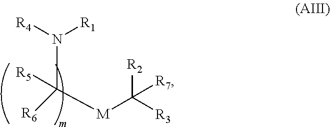

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R⁴ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂).N(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R⁴ is —(CH₂)ₙQ in which n is 1 or 2, or (ii) R⁴ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R² and R³ are independently selected from the group consisting of H, C₁₋₁₄ alkyl, C₂₋₁₄ alkenyl, —R*YR″, —YR″, and —R*OR″, or R² and R³, together with the atom to which they are attached, form a heterocycle or carbocycle;
R⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R² and R³, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R² and R³, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (AIII) includes those of Formula (AIII-A):

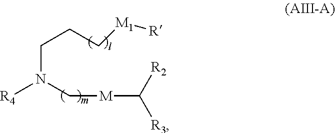

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is —OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R

In certain embodiments, a subset of compounds of Formula (AIII) includes those of Formula (AIII-B):

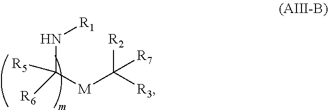

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R

In certain embodiments, a subset of compounds of Formula (AIII) includes those of Formula (AIII-C):

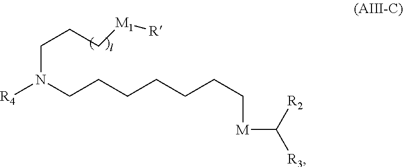

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (AIII) are of Formula (AIII-D),

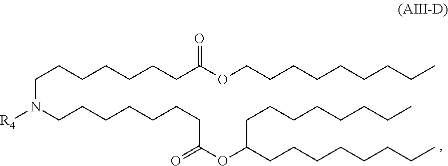

or their N-oxides, or salts or isomers thereof, wherein R₄ is as described herein.

In another embodiment, the compounds of Formula (AIII) are of Formula (AIII-E),

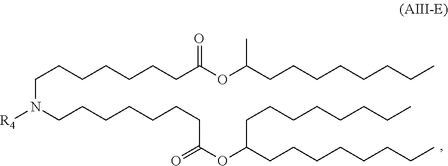

or their N-oxides, or salts or isomers thereof, wherein R₄ is as described herein.

In another embodiment, the compounds of Formula (AIII) are of Formula (AIII-F) or (AIII-G):

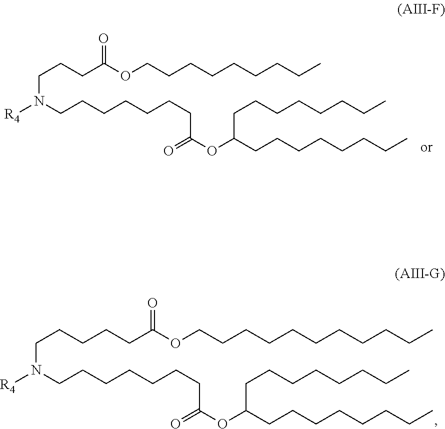

or their N-oxides, or salts or isomers thereof, wherein R₄ is as described herein.

In another embodiment, the compounds of Formula (AIII) are of Formula (AIII-H):

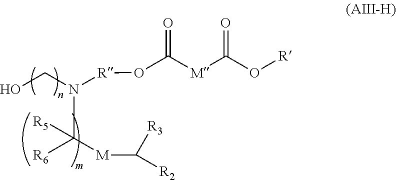

or their N-oxides, or salts or isomers thereof, wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

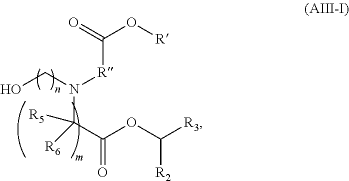

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid comprises a compound having structure:

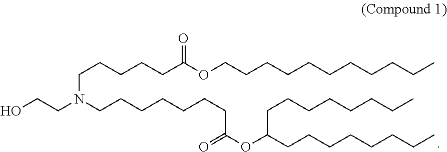 [SMILES: CCCCCCCCCCCOC(=O)CCCCCN(CCO)CCCCCCCC(=O)OC(CCCCCCCC)CCCCCCCC]

In some embodiments, an ionizable amino lipid comprises a compound having structure:

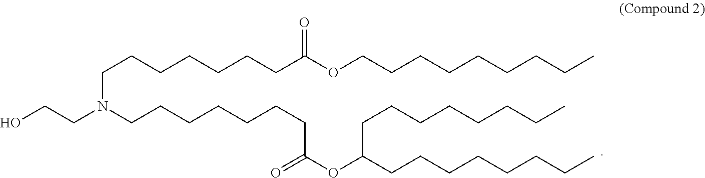 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

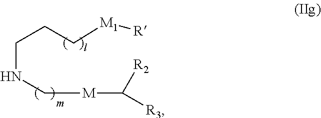

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids of a lipid nanoparticle may be one or more of compounds of formula (AIV),

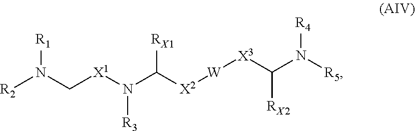

or salts or isomers thereof, wherein
W is

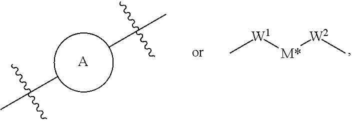

ring A is

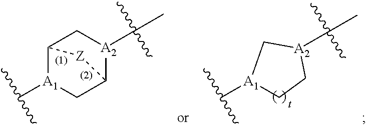

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S), and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

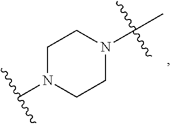 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (AIVa)-(AIVh):

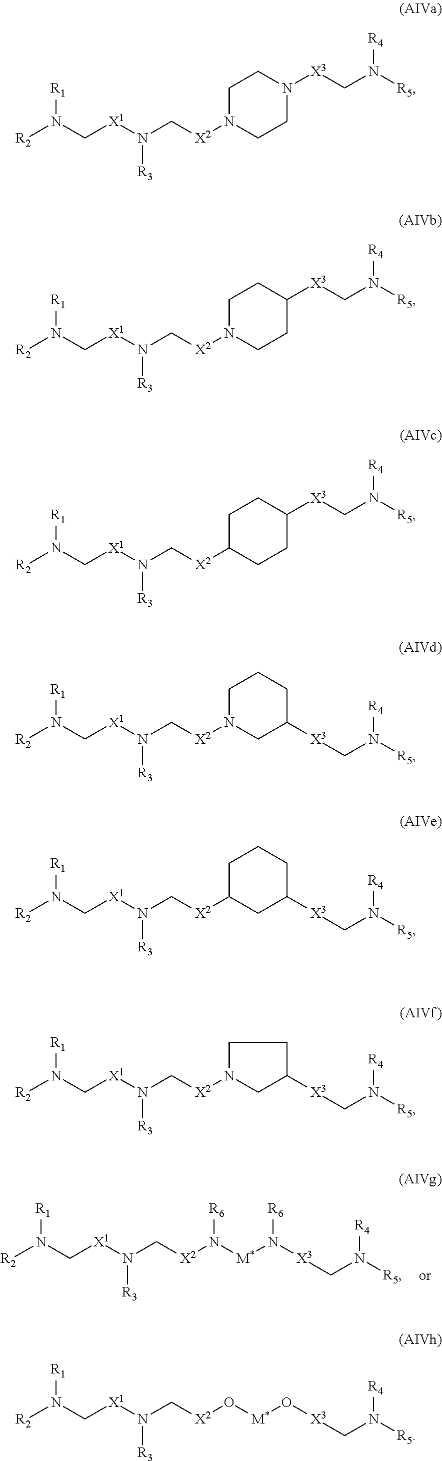

In some embodiments, the ionizable amino lipid is

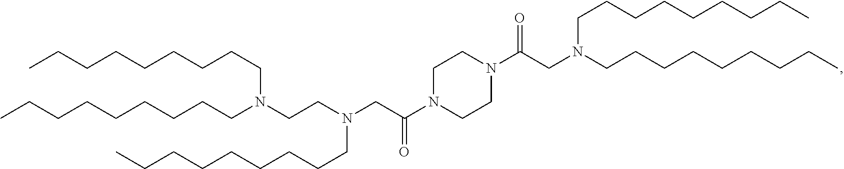 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

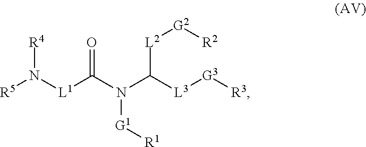

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

### Formula (AVI)

In some embodiments, the lipid nanoparticle comprises a lipid having the structure:

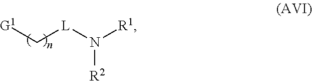

or a pharmaceutically acceptable salt, tautomer, or stereoisomer thereof, wherein:
G¹ is —N(R³)R⁴ or —OR;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

### Formula (AVII)

In some embodiments, the lipid nano article comprises a lipid having the structure:

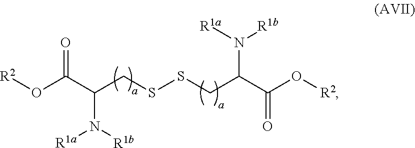

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each L₁ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and each c is independently an integer of 1-10.

### Formula (AVIII)

In some embodiments, the lipid nanoparticle comprises a lipid having the structure:

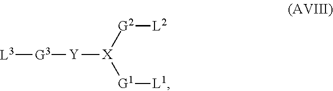

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{c} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R₂ and R₃ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

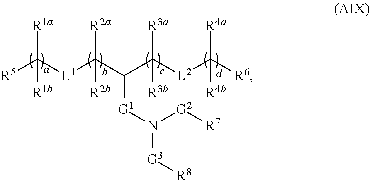

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R₇ is H or C₁-C₂₀ alkyl;
R⁸ is OH, —N(R⁹)(C═O)R¹⁰, —(C═O)NR⁹R¹⁰, —NR⁹R¹⁰, —(C═O)OR″ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

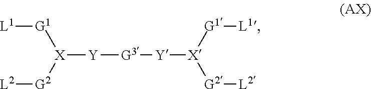

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²;
G¹. G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

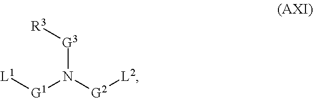

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R_{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R₄ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

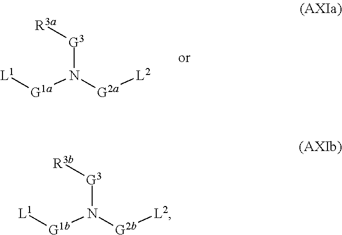

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{a4} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R₆ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

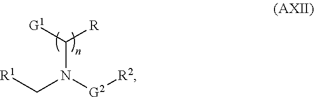

or a pharmaceutically acceptable salt, prodrug or stereoisomer thereof, wherein:
G¹ is —OH, —R³R⁴, —(C═O)R⁵ or —R³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

### Formula (AXII)

In some embodiments, the lipid nanoparticle comprises a lipid having the structure:

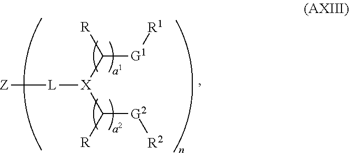

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

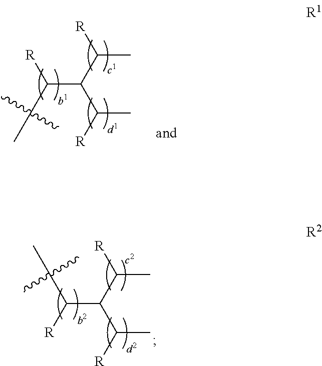

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

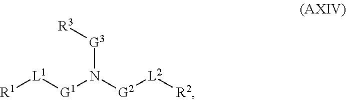

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR, CN, —C(═O)OR⁴, —OC(═O)R⁴ or —R⁵C(═O)R⁴;
R₄ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

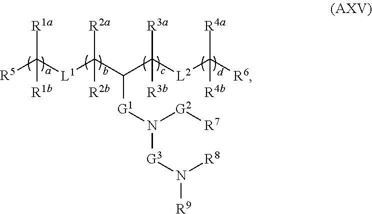

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond:
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

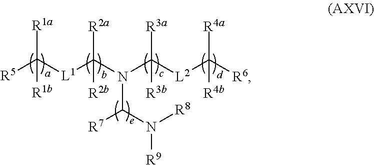

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R₄ₐ is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

### Formula (AXVII)

In some embodiments, the lipid nanoparticle comprises a lipid having the structure:

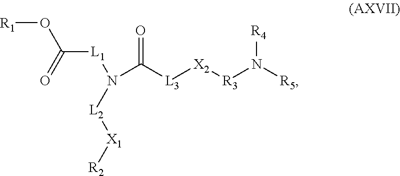

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

### Compounds (A1)-(A11)

In some embodiments, the lipid nanoparticle comprises an ionizable lipid having the structure:

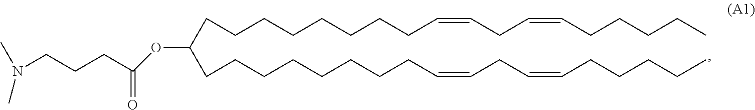 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

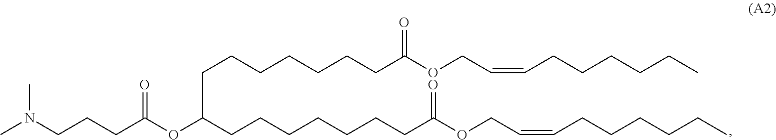 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

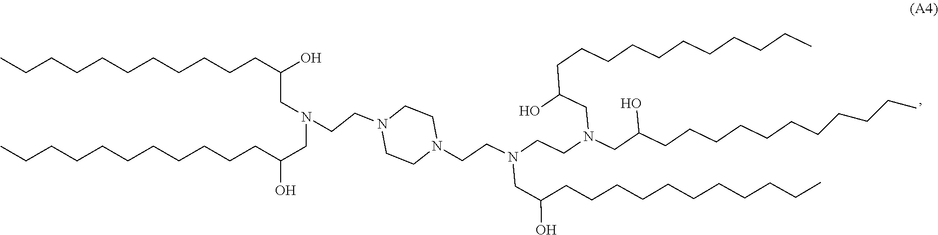 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

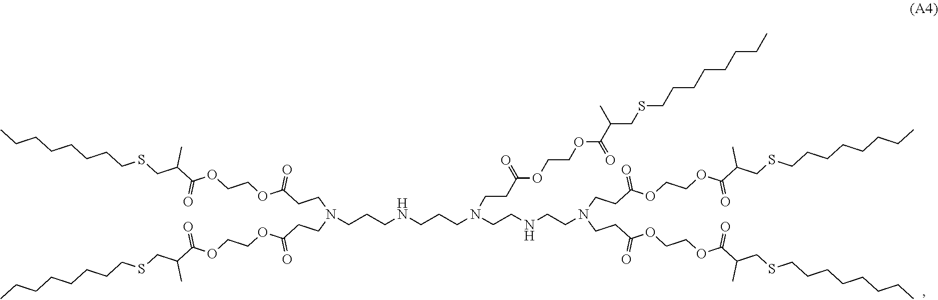 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, the lipid nanoparticle comprises a lipid having the structure:

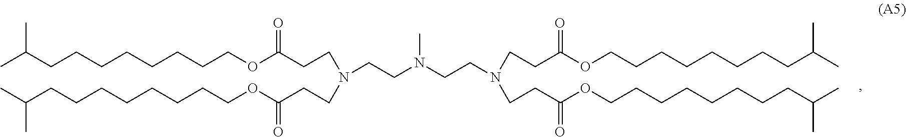 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

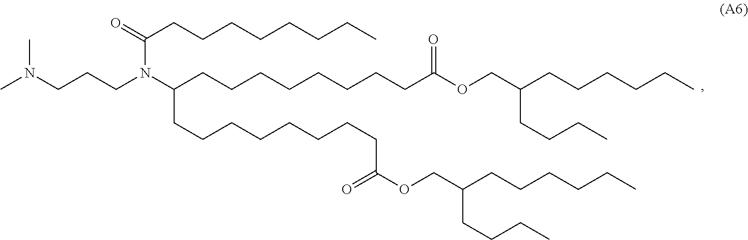 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

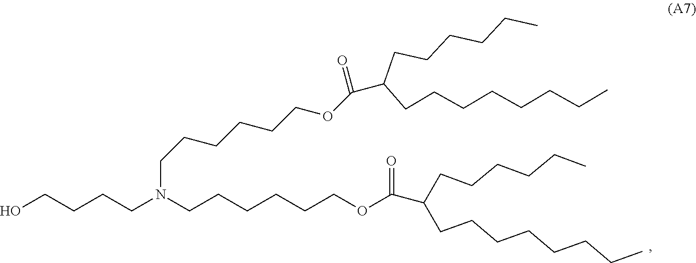 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

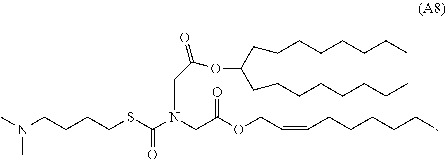

or a pharmaceutically acceptable salt thereof.

In some embodiments, the lipid nanoparticle comprises a lipid having the structure:

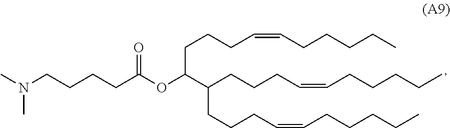 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

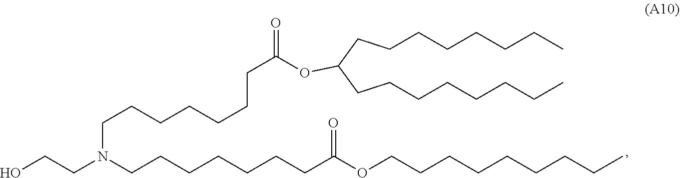 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

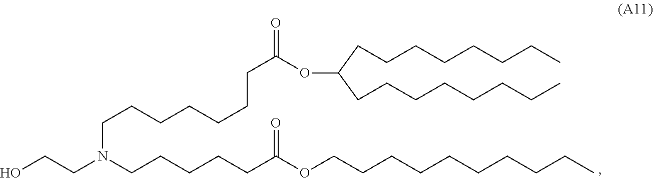 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, lipid nanoparticles comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of a lipid nanoparticle composition can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

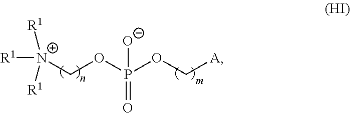

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3,4, 5, 6, 7, 8, 9, or 10;
A is of the formula:

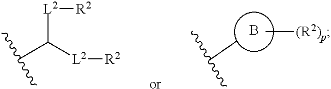

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

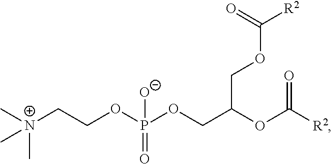

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmitoleoyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG2&-DMG.

In some embodiments, lipid nanoparticles can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

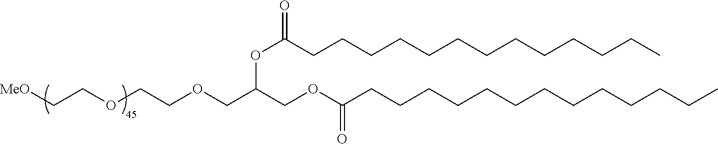

In some embodiments, PEG lipids can be PEGylated lipids described in International Publication No. WO2012099755, the contents of which is herein incorporated by reference in its entirety. Any of these exemplary PEG lipids may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG lipid is a compound of Formula (PI):

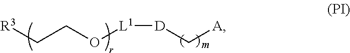

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9 or 10;
A is of the formula:

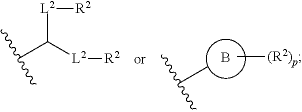

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI-OH):

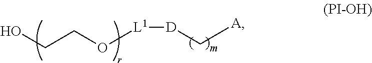

or a salt thereof.

### Formula (PII)

In certain embodiments, a PEG lipid is a PEGylated fatty acid. In certain embodiments, a PEG lipid is a compound of Formula (PII). In some embodiments, compounds of Formula (PII) have the following formula:

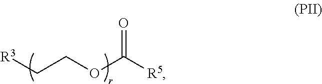

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(O)O, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PII) is of Formula (PII-OH):

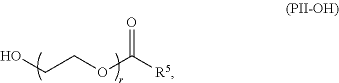

or a salt thereof. In some embodiments, r is 40-50.

In et other embodiments the compound of Formula (PII) is:

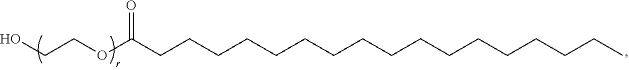

or a salt thereof.

In some embodiments, the compound of Formula (PII) is

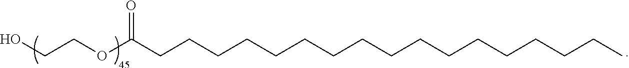

In some embodiments, the lipid composition of a pharmaceutical composition does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG- lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP comprises an ionizable amino lipid of Compound 1, wherein the non-cationic lipid is DSPC, the structural lipid that is cholesterol, and the PEG lipid is DMG-PEG.

In some embodiments, a LNP comprises an ionizable amino lipid of Compound 2, wherein the non-cationic lipid is DSPC, the structural lipid that is cholesterol, and the PEG lipid is DMG-PEG.

In some embodiments, a LNP comprises an ionizable amino lipid of any of Formula (AIII), (AIV), or (AV), a phospholipid comprising DSPC, a structural lipid, and a PEG lipid comprising PEG-DMG.

In some embodiments, a LNP comprises an ionizable amino lipid of any of Formula (AIII), (AIV), or (AV), a phospholipid comprising DSPC, a structural lipid, and a PEG lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG lipid comprising a compound having Formula (PII).

In some embodiments, the lipid nanoparticle comprises 49 mol % ionizable amino lipid, 10 mol % DSPC, 38.5 mol % cholesterol, and 2.5 mol % DMG-PEG.

In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some aspects, a lipid composition may comprise one or more lipids. Such lipids may include those useful in the preparation of lipid nanoparticle formulations as described above or as known in the art.

### Stabilizing Compounds

Some embodiments of the compositions are stabilized pharmaceutical compositions. Various non-viral delivery systems, including nanoparticle formulations, present attractive opportunities to overcome many challenges associated with mRNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

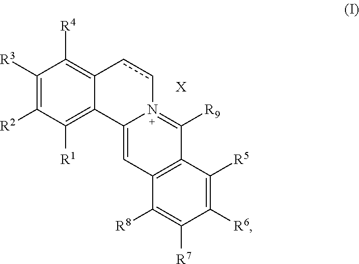

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R₄ is H; R⁵ is H or OCH₃; R₆ is OCH₃; R₇ is H or OCH₃; R⁸ is H; R₉ is H or CH₃; and X is a pharmaceutically acceptable anion, e.g., a halide such as chloride.

In some embodiments, the compound of Formula (I) has the structure of:

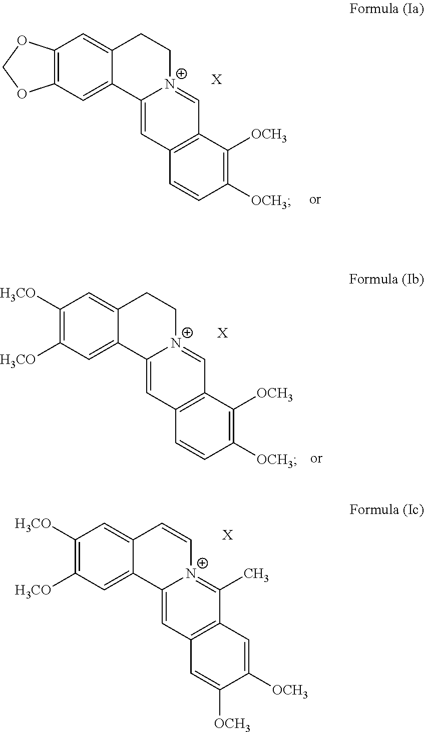

or a tautomer or solvate thereof.

In some embodiments, the stabilized pharmaceutical composition comprises a nucleic acid formulation comprising a nucleic acid and a lipid, and a compound of Formula (II):

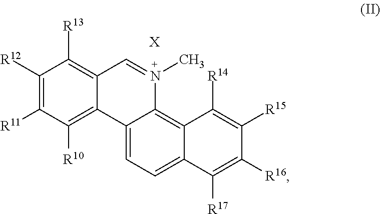

or a tautomer or solvate thereof, wherein:
R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

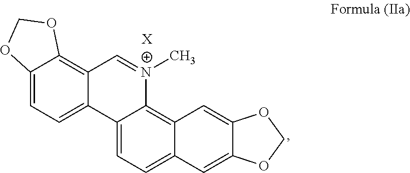

or a tautomer or solvate thereof.

Stabilizing compounds of Formulas (I), (Ia), (Ib), (Ic), (II), and (Iia) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10⁻³ M (e.g., less than 10⁻⁴ M, less than 10⁻⁵ M, less than 10⁻⁵ M, less than 10⁻⁷ M, less than 10⁻⁸ M, or less than 10⁻⁹ M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 24 μM, 36 μM, 4 μM, 59 μM, 6 μM, 7 μM, 8 μM, 9 μM, 10 μM, 15 μM, 20 μM, 25 μM, 30 μM, 35 μM, 40 μM, 45 M, 50 μM, 60 μM, 70 M, 80 μM, 90 μM, or 100 μM.

### Multivalent Vaccines

The compositions (e.g. vaccines) may include RNA or multiple RNAs encoding two or more antigens of the same or different species. In some embodiments, composition includes an RNA or multiple RNAs encoding two or more coronavirus antigens. In some embodiments, the RNA may encode 1, 2, 3, 4, 5, 6, 7, 8, 9, or 10, or more coronavirus antigens.

In some embodiments, two or more different mRNA encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately. In some embodiments, when the composition comprises two different RNA encoding antigens, the ratio of RNA encoding antigens is 1:1, 1:2, 1:4, 4:1, or 2:1.

### Initial or First Vaccine

In some embodiments the first or initial vaccine is an mRNA vaccine encoding a 2P stabilized spike protein. For instance, the initial or first vaccine may be an mRNA encoding a spike antigen having an amino acid sequence of SEQ ID NO: 5. In other embodiments the first vaccine may be any vaccine modality comprising a 2P stabilized spike protein. As a non-limiting example, the first vaccine composition may be a recombinant vaccine. As used herein, “recombinant vaccine” refers to a vaccine made by genetic engineering, the process and method of manipulating the genetic material of an organism. In most cases, a recombinant vaccine encompasses one or more nucleic acids encoding protein antigens that have either been produced and purified in a heterologous expression system (e.g., bacteria or yeast) or purified from large amounts of the pathogenic organism. Following administration, a vaccinated person produces antibodies to the one or more protein antigens, thus protecting him/her from disease. In some embodiments, the recombinant vaccine is a vectored vaccine. Viral vectored vaccines comprise a polynucleotide sequence not of viral origin (i.e., a polynucleotide heterologous to the virus), that encodes a peptide, polypeptide, or protein capable of eliciting an immune response in a host contacted with the vector. Expression of the polynucleotide results in the generation of a neutralizing antibody response and/or a cell mediated response, e.g., a cytotoxic T cell response. Examples of viral vectored vaccines include, but are not limited to, those developed by Oxford/AstraZeneca (COVID-19 Vaccine AstraZeneca), CanSino Biological Inc./Beijing Institute of Biotechnology, Gamaleya Research Institute, Zydus Cadila, Institut Pasteur/Themis/University of Pittsburgh Center for Vaccine Research, University of Hong Kong, and Altimmune (NasoVAX). In some embodiments, the recombinant vaccine is a nucleic acid-based (e.g., DNA, mRNA) coronavirus vaccine. Exemplary DNA vaccines include those being developed by Inovio Pharmaceuticals (INO-4800), Genexine Consortium (GX-19), OncoSec and the Cancer Institute (CORVax12 and TAVO™), Karolinska Institute/Cobra Biologics, Osaka University/Anges/Takara Bio, and Takis/Applied DNA Sciences/Evvivax. Exemplary mRNA vaccines include those being developed by BioNTech/Pfizer, Imperial College London, Curevac, and Walvax Biotech/People's Liberation Army (PLA) Academy of Military Science.

### Pharmaceutical Formulations

In some embodiments, compositions (e.g., pharmaceutical compositions), methods, kits and reagents are used for prevention or treatment of coronavirus in humans and other mammals, for example. Compositions can be used as therapeutic or prophylactic agents. They may be used in medicine to prevent and/or treat a coronavirus infection.

In some embodiments, the SARS-CoV-2 vaccine containing RNA can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modification are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) may be used for treatment or prevention of a coronavirus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the vaccine composition and may include a traditional boost, seasonal boost or a pandemic shift boost. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, or 6 months, 7 months, 8 months, 9 months, 10 months, 11 months, one year, or more. In some embodiments, the time of administration between the initial administration of the prophylactic composition and the booster is at least 6 months. In exemplary embodiments, the time of administration between the initial administration of the prophylactic composition and the booster may be, but is not limited to, 1 week, 2 weeks, 3 weeks, 1 month, 2 months, 3 months, or 6 months. A booster may comprise the same or different mRNAs as compared to the earlier administration of the prophylactic composition. In some embodiments, the booster may comprise a combination of the same mRNA from the earlier administration of the prophylactic composition and at least one different mRNA. In some embodiments, the ratio of the mRNA from the earlier administration of the prophylactic composition and the at least one different mRNA is 1:1, 1:2, 1:4, 4:1, or 2:1. In one embodiment, the ratio is 1:1. In some embodiments, the booster may comprise different mRNAs as compared to the earlier administration of the prophylactic compositions. In some embodiments, such a booster may comprise 1, 2, 3, 4 or more mRNAs that were not present in the prophylactic composition. In some embodiments, the ratio of two mRNA polynucleotides (none of which were in the prophylactic composition) in the booster is 1:1, 1:2, 1:4, 4:1, or 2:1. In one embodiment, the ratio is 1:1. A boost or booster dose may be administered more than once, for example 2, 3, 4, 5, 6 or more times after the initial prophylactic (prime) dose. In some embodiments, a subsequent boost is administered within weeks, e.g., within 3-4 weeks of the first (or previous) boost. In some embodiments, a second boost is administered 1, 2, 3, 4, 5, 6, 7, 8, 9, 10, 11, 12, or more weeks after the first (or previous) boost. The booster, in some embodiments is monovalent (e.g., the mRNA encodes a single antigen). In some embodiments, the booster is multivalent (e.g., the mRNA encodes more than one antigen).

In some embodiments, the booster dose (that is, the total dose administered) is 1 μg-5 μg, 1 μg-10 μg, 1 μg-15 μg, 1 μg-20 μg, 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the booster dose is 10 μg-60 μg, 10 μg-55 μg, 10 μg-50 μg, 10 μg-45 μg, 10 μg-40 μg, 10 μg-35 μg, 10 μg-30 μg, 10 μg-25 μg, 10 μg-20 μg, 15 μg-60 μg, 15 μg-55 μg, 15 μg-50 μg, 15 μg-45 μg, 15 μg-40 μg, 15 μg-35 μg, 15 μg-30 μg, 15 μg-25 μg, 15 μg-20 μg, 20 μg-60 μg, 20 μg-55 μg, 20 μg-50 μg, 20 μg-45 μg, 20 μg-40 μg, 20 μg-35 μg, 20 μg-30 μg, 20 μg-25 μg, 25 μg-60 μg, 25 μg-55 μg, 25 μg-50 μg, 25 μg-45 μg, 25 μg-40 μg, 25 μg-35 μg, 25 μg-30 μg, 30 μg-60 μg, 30 μg-55 μg, 30 μg-50 μg, 30 μg-45 μg, 30 μg-40 μg, 30 μg-35 μg, 35 μg-60 μg, 35 μg-55 μg, 35 μg-50 μg, 35 μg-45 μg, 35 μg-40 μg, 40 μg-60 μg, 40 μg-55 μg, 40 μg-50 μg, 40 μg-45 μg, 45 μg-60 μg, 45 μg-55 μg, 45 μg-50 μg, 50 μg-60 μg, 50 μg-55 μg, or 55 μg-60 μg. In some embodiments, the booster dose is at least 10 μg and less than 25 μg of the composition. In some embodiments, the booster dose is at least 5 μg and less than 25 μg of the composition. For example, the booster dose is 1 μg, 2 μg, 2.5 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the booster dose is 50 μg.

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

In some embodiments, pharmaceutical compositions include RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (it is adjuvant free).

An RNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. As used herein, the phrase “active ingredient” generally refers to the RNA vaccines or the polynucleotides contained therein, for example, RNA polynucleotides (e.g., mRNA polynucleotides) encoding antigens.

Formulations of the vaccine compositions may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Immunizing compositions (e.g., RNA vaccines), methods, kits and reagents may be used for prevention and/or treatment of coronavirus infection in humans and other mammals. Immunizing compositions can be used as therapeutic or prophylactic agents. In some embodiments, immunizing compositions are used to provide prophylactic protection from coronavirus infection. In some embodiments, immunizing compositions are used to treat a coronavirus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA vaccine) is administered to a subject (e.g., a mammalian subject, such as a human subject) in an effective amount to induce an antigen-specific immune response. The RNA encoding the coronavirus spike protein antigen is expressed and translated in vivo to produce the antigen, which then stimulates an immune response in the subject.

Prophylactic protection from a coronavirus can be achieved following administration of an immunizing composition (e.g., an mRNA vaccine). Immunizing compositions can be administered once, twice, three times, four times or more but it is likely sufficient to administer the vaccine once (optionally followed by a single booster). It is possible, although less desirable, to administer an immunizing composition to an infected individual to achieve a therapeutic response. Dosing may need to be adjusted accordingly.

In some aspects, methods of eliciting an immune response in a subject against a coronavirus antigen (or multiple antigens) comprise administration of an immunizing composition (e.g. an mRNA vaccine) to a subject. In some embodiments, a method involves administering to the subject an immunizing composition comprising a mRNA having an open reading frame encoding a coronavirus antigen, thereby inducing in the subject an immune response specific to the coronavirus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than mRNA vaccines. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the coronavirus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the coronavirus or an unvaccinated subject.

Methods of eliciting an immune response in a subject against a coronavirus may comprise administration of an immunizing composition (e.g. mRNA vaccine) to a subject. The method involves administering to the subject a composition comprising an mRNA comprising an open reading frame encoding a coronavirus antigen, thereby inducing in the subject an immune response specific to the coronavirus, wherein the immune response in the subject is equivalent to an immune response in a subject vaccinated with a traditional vaccine against the coronavirus at 2 times to 100 times the dosage level relative to the composition.

In some embodiments, the immune response in the subject is equivalent to an immune response in a subject vaccinated with a traditional vaccine at twice the dosage level relative to an mRNA vaccine. In some embodiments, the immune response in the subject is equivalent to an immune response in a subject vaccinated with a traditional vaccine at three times the dosage level relative to an mRNA vaccine. In some embodiments, the immune response in the subject is equivalent to an immune response in a subject vaccinated with a traditional vaccine at 4 times, 5 times, 10 times, 50 times, or 100 times the dosage level relative to an mRNA vaccine. In some embodiments, the immune response in the subject is equivalent to an immune response in a subject vaccinated with a traditional vaccine at 10 times to 1000 times the dosage level relative to an mRNA vaccine. In some embodiments, the immune response in the subject is equivalent to an immune response in a subject vaccinated with a traditional vaccine at 100 times to 1000 times the dosage level relative to an mRNA vaccine.

In other embodiments, the immune response is assessed by determining [protein] antibody titer in the subject. In other embodiments, the ability of serum or antibody from an immunized subject is tested for its ability to neutralize viral uptake or reduce coronavirus transformation of human B lymphocytes. In other embodiments, the ability to promote a robust T cell response(s) is measured using art recognized techniques.

In some embodiments, a method comprises eliciting an immune response in a subject against a coronavirus by administering to the subject composition comprising an mRNA having an open reading frame encoding a coronavirus antigen, thereby inducing in the subject an immune response specific to the coronavirus antigen, wherein the immune response in the subject is induced 2 days to 10 weeks earlier relative to an immune response induced in a subject vaccinated with a prophylactically effective dose of a traditional vaccine against the coronavirus. In some embodiments, the immune response in the subject is induced in a subject vaccinated with a prophylactically effective dose of a traditional vaccine at 2 times to 100 times the dosage level relative to an mRNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

In some embodiments, methods of eliciting an immune response in a subject against a coronavirus comprise administering to the subject an mRNA having an open reading frame encoding a first antigen, wherein the RNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. In some embodiments, methods comprise administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount (e.g., effective dose) of the RNA may be as low as 10 μg, administered for example as a single dose or as two 5 μg doses. In some embodiments, the effective amount (e.g., effective dose) is a total dose of 5 μg-300 μg, for example, 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, 25 μg-50 μg, 25 μg-150 μg, 25 μg-200 μg, 25 μg-250 μg, or 25 μg-300 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 2.5 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg. Any of the doses provided above may be an effective amount for a booster dose; for example, in some embodiments, the booster dose is a total dose of 50 μg. In some embodiments, the composition comprises two or more mRNA polynucleotides and effective amount is a total dose of 20 μg (e.g., 10 μg of a first mRNA and 10 μg of a second mRNA). In some embodiments, the composition comprises two or more mRNA polynucleotides and effective amount is a total dose of 50 μg (e.g., 25 μg of a first mRNA and 25 μg of a second mRNA). In some embodiments, the composition comprises two or more mRNA polynucleotides and effective amount is a total dose of 100 μg (e.g., 50 μg of a first mRNA and 50 μg of a second mRNA).

RNAs can be formulated into a suitable dosage form, such as an intranasal, intratracheal, or injectable (e.g., intravenous, intraocular, intravitreal, intramuscular, intradermal, intracardiac, intraperitoneal, and subcutaneous).

### Vaccine Efficacy

In some aspects, compositions (e.g., RNA vaccines) comprise RNA formulated in an effective amount to produce an antigen specific immune response in a subject (e.g., production of antibodies specific to a coronavirus antigen). “An effective amount” is a dose of the RNA effective to produce an antigen-specific immune response. In some embodiments, compositions are used in methods of inducing an antigen-specific immune response in a subject.

As used herein, an “immune response” to a vaccine or LNP is the development in a subject of a humoral and/or a cellular immune response to a (one or more) coronavirus protein(s) present in the vaccine. As used herein, “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-coronavirus antigen antibody titer produced in a subject administered a composition (e.g. vaccine). An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, SAR-CoV-2 virus or SAR-CoV-2 viral antigen, e.g., SAR-CoV-2 spike or S protein, of domain thereof. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection.

The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding SARS-CoV-2 neutralizing antibodies, the “seroprotective” threshold titer remains unknown; but a seropositivity threshold of 1:10 can be considered a seroprotection threshold in certain embodiments.

In some embodiments a neutralizing immune response is an immune response that produces a level of antibodies that meet or exceed a seroprotection threshold.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. An antibody titer may be used to determine the strength of an immune response induced in a subject by a composition (e.g., RNA vaccine).

In some embodiments, an anti-coronavirus antigen antibody titer produced in a subject is increased by at least 1 log relative to a control. For example, anti-coronavirus antigen antibody titer produced in a subject may be increased by at least 1.5, at least 2, at least 2.5, or at least 3 log relative to a control. In some embodiments, the anti-coronavirus antigen antibody titer produced in the subject is increased by 1, 1.5, 2, 2.5 or 3 log relative to a control. In some embodiments, the anti-coronavirus antigen antibody titer produced in the subject is increased by 1-3 log relative to a control. For example, the anti-coronavirus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-coronavirus antigen antibody titer produced in a subject is increased at least 2 times relative to a control. For example, the anti-coronavirus antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-coronavirus antigen antibody titer produced in the subject is increased 2, 3, 4, 5, 6, 7, 8, 9, or 10 times relative to a control. In some embodiments, the anti-coronavirus antigen antibody titer produced in a subject is increased 2-10 times relative to a control. For example, the anti-coronavirus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to coronavirus. A geometric mean titer (GMT) is the average antibody titer for a group of subjects calculated by multiplying all values and taking the nth root of the number, where n is the number of subjects with available data.

A control, in some embodiments, is an anti-coronavirus antigen antibody titer produced in a subject who has not been administered a composition (e.g., RNA vaccine). In some embodiments, a control is an anti-coronavirus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of a composition (e.g., RNA vaccine) to be effective is measured in a murine model. For example, a composition may be administered to a murine model and the murine model assayed for induction of neutralizing antibody titers. Viral challenge studies may also be used to assess the efficacy of a vaccine. For example, a composition may be administered to a murine model, the murine model challenged with virus, and the murine model assayed for survival and/or immune response (e.g., neutralizing antibody response, T cell response (e.g., cytokine response)).

In some embodiments, an effective amount of a composition (e.g., RNA vaccine) is a dose that is reduced compared to the standard of care dose of a recombinant protein vaccine. A “standard of care,” as used herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as used herein, refers to the dose of a recombinant or purified protein vaccine, or a live attenuated or inactivated vaccine, or a VLP vaccine, that a physician/clinician or other medical professional would administer to a subject to treat or prevent coronavirus infection or a related condition, while following the standard of care guideline for treating or preventing coronavirus infection or a related condition.

In some embodiments, the anti-coronavirus antigen antibody titer produced in a subject administered an effective amount of a composition is equivalent to an anti-coronavirus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ; and$$

$$Efficacy = ( 1 - RR ) \times 100.$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 100.$$

In some embodiments, efficacy of the composition (e.g., RNA vaccine) is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition is sufficient to provide sterilizing immunity in the subject for at least 1 year. For example, the effective amount of a composition is sufficient to provide sterilizing immunity in the subject for at least 2 years, at least 3 years, at least 4 years, or at least 5 years. In some embodiments, the effective amount of a composition is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition is sufficient to produce detectable levels of coronavirus antigen as measured in serum of the subject at 1-72 hours post administration.

Titer. An antibody titer is a measurement of the number of antibodies within a subject, for example, antibodies that are specific to a particular antigen (e.g., an anti-coronavirus antigen). Antibody titer is typically expressed as the inverse of the greatest dilution that provides a positive result. Enzyme-linked immunosorbent assay (ELISA) is a common assay for determining antibody titers, for example.

A neutralizing immune response is an immune response that is a neutralizing antibody response and/or an effective neutralizing T cell response. In some embodiments a neutralizing antibody response produces a level of antibodies that meet or exceed a seroprotection threshold.

An effective T cell response is a response which produces a baseline level of viral activated or viral specific T cells including CD8+ and CD4+ T helper type 1 cells. CD8+ cytotoxic T lymphocytes typically clear the intracellular virus compartment and CD4+ T cells exert various functions in the body such as helping B and other T cells, promoting memory generation and indirect or direct cytotoxic activity. In some embodiments the effective T cells comprises a high proportion of CD8+ T cells and/or CD4+ T cells, relative to a baseline level (in a naïve subject). In some embodiments these T cells are differentiated towards an early-differentiated memory phenotype with co-expression of CD27 and CD28.

In some embodiments, the effective amount of a composition is sufficient to produce a 1,000-10,000 neutralizing antibody titer produced by neutralizing antibody against the coronavirus antigen as measured in serum of the subject at 1-72 hours post administration. In some embodiments, the effective amount is sufficient to produce a 1,000-5,000 neutralizing antibody titer produced by neutralizing antibody against the coronavirus antigen as measured in serum of the subject at 1-72 hours post administration. In some embodiments, the effective amount is sufficient to produce a 5,000-10,000 neutralizing antibody titer produced by neutralizing antibody against the coronavirus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀. In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-coronavirus antigen antibody titer produced in the subject is increased by at least 1 log relative to a control. For example, an anti-coronavirus antigen antibody titer produced in the subject may be increased by at least 2, 3, 4, 5, 6, 7, 8, 9 or 10 log relative to a control.

In some embodiments, an anti-coronavirus antigen antibody titer produced in the subject is increased at least 2 times relative to a control. For example, an anti-coronavirus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### EXAMPLES

### Example 1. In Vitro Expression of BA.4/BA.5 mRNA Contained in the Bivalent mRNA-1273.222 Vaccine

Expi293 cells were transiently transfected at a density of 10⁶ cells/mL with BA.4/BA.5 mRNA contained in mRNA-1273.222 (SEQ ID NO: 1 and SEQ ID NO: 9 [ORF SEQ ID NO: 10, encoding SEQ ID NO: 12] at a 1:1 ratio) or mRNA contained in mRNA-1283 (mRNA encoding a receptor-binding domain and N-terminal domain of the SARS-CoV-2 spike protein; positive control) using a TransIT®-mRNA transfection kit (Mirus) at the concentrations to be tested in the assays (500 ng/mL and 100 ng/mL). Mock transfected cells (i.e., mock mRNA) were used as control. Cell incubation conditions were kept according to the user manual specifications (ThermoFisher Scientific).

After a 48-hour incubation period, the cells were collected and resuspended in Flow Cytometry Staining Buffer (1×PBS, 2% fetal bovine serum). The cells were then blocked using PBS with 5% goat serum for 20 minutes at 4° C. To detect surface protein expression, the cells were stained with a 1:1000 dilution of the mAb CR3022 (Genscript), a 1:1000 dilution of the mAb CC40.8 (Genscript), and 5 μg/mL of the recombinant hACE-2 (Moderna) in Flow Cytometry Staining Buffer for 1 hour, at 4° C.

The mAb CC40.8 is specific for the NTD of both the original Wuhan-Hu-1 spike as well as all variants that have emerged to date and binds to a conserved region on the S2 subdomain. The mAb CR3022 was included in this analysis as this antibody selectively binds to the original Wuhan-Hu-1 spike protein in the RBD. A recombinant hACE-2 was used to measure expression as hACE-2 will bind to the RBM in the original Wuhan-Hu-1 spike as well as all variants that have emerged to date. The mRNA contained in mRNA-1283 was used as a positive control as it is known to bind to the CR3022 mAb.

Following staining, the Expi293 cells were washed twice in Flow Cytometry Staining Buffer and incubated with Alexa Fluor 647-goat anti-human immunoglobulin G (Southern Biotech) in Flow Cytometry Staining Buffer for 30 minutes at 4° C. Live/Dead aqua fixable stain (Invitrogen) was used to assess viability using a 1:1000 dilution in Flow Cytometry Staining Buffer. Cells were then fixed using BD Cytofix Fixation Buffer (ThermoFisher Scientific). Data acquisition was performed, and raw data analyzed on a Sartorius Intellicyt® iQue3 instrument.

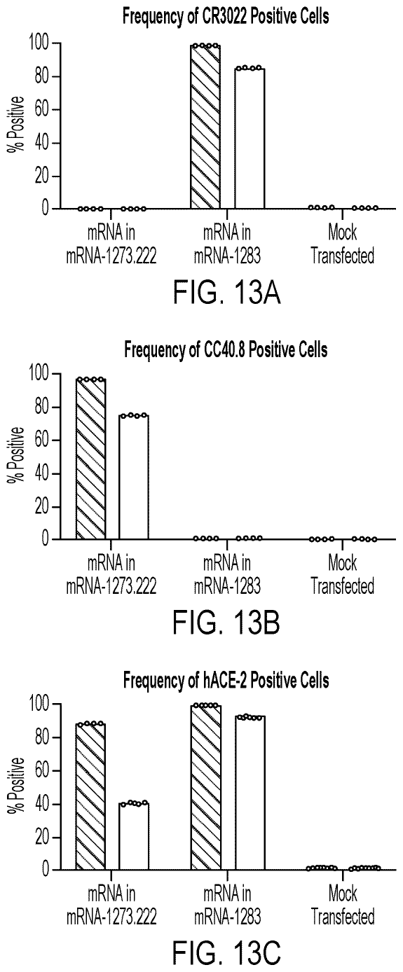

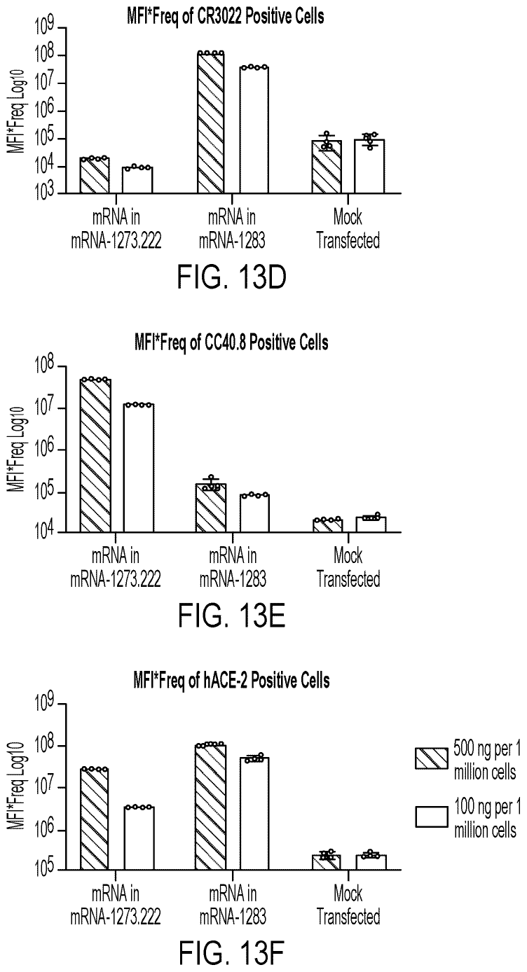

The frequency and intensity of expression of BA.4/BA.5 mRNA contained in mRNA-1273.222 and the mRNA contained in mRNA-1283 (positive control) is shown in FIGS. 13A-13F. After 48 hours, the cells were collected and assessed for expression of the encoded antigen. In vitro evaluation of cell surface expression in cells transfected with a dose concentration of 500 ng/mL and 100 ng/mL mRNA (BA.4/BA.5 mRNA contained in mRNA-1273.222 and mRNA contained in mRNA-1283) revealed substantial and dose-dependent expression on the cell surface of each encoded antigen, with antibody-binding patterns that are expected given the binding selectivity of each detection reagent.

The BA.4/BA.5 mRNA contained in mRNA-1273.222 was detected in the majority of cells assessed at high MFIs by the mAb that binds to a conserved region on the S2 subdomain (CC40.8; FIGS. 13B, 13E) and with the recombinant hACE-2 (FIGS. 13C, 13F). No detection of this antigen was measured with the CR3022 mAb (FIGS. 13A, 13D), as expected, given that the binding epitope is not present on the BA.4/BA.5 spike. The mRNA contained in mRNA-1283 (positive control) was detected by the CR3022 mAb and recombinant hACE-2, but not the CC40.8 mAb (as expected), and confirmed the binding ability of the CR3022 mAb to the RBD of the Wuhan-Hu-1 spike. A small dose effect was observed overall.

The MFI of expression was similar to the observed frequency in cells transfected with BA.4/BA.5 mRNA contained in mRNA-1273.222. Cells that did not undergo transfection (mock-transfected cells) had little to no change.

### Example 2. Immunogenicity and Protection from Booster Dose of mRNA-1273.222 Vaccine in Mice

### K18-hACE2 Mice

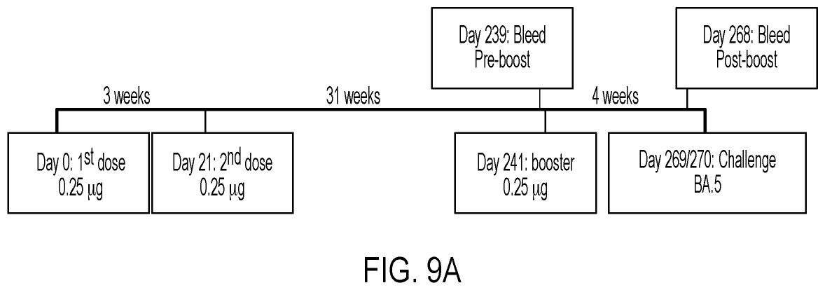

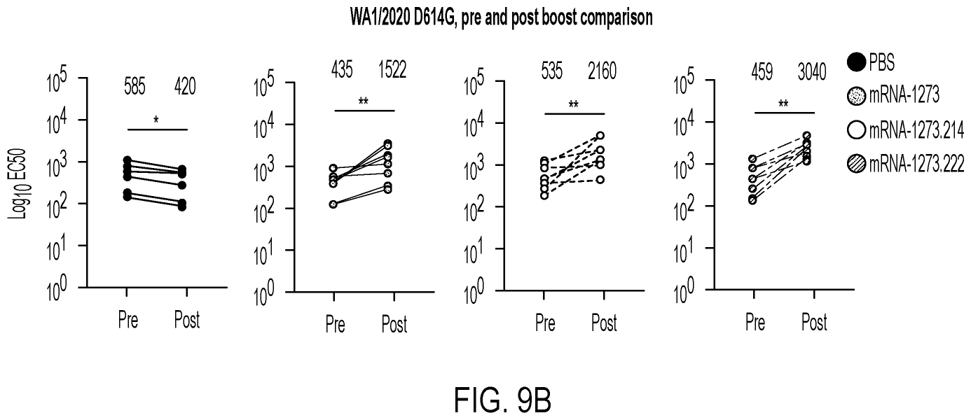

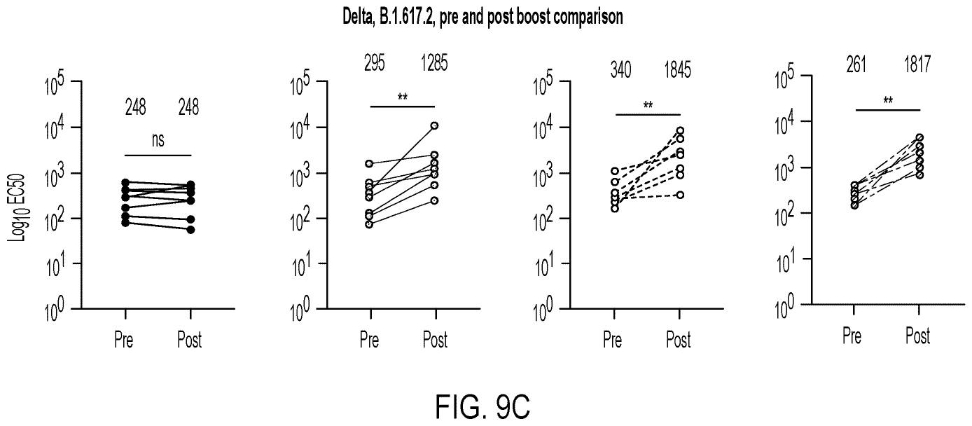

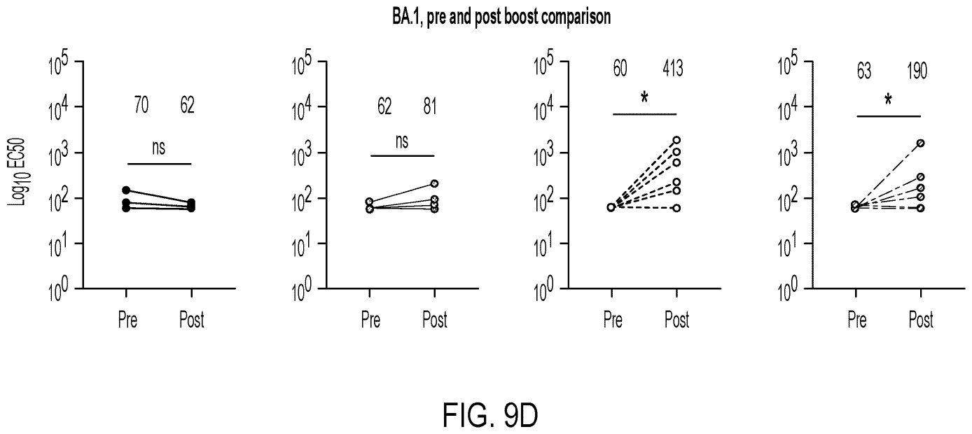

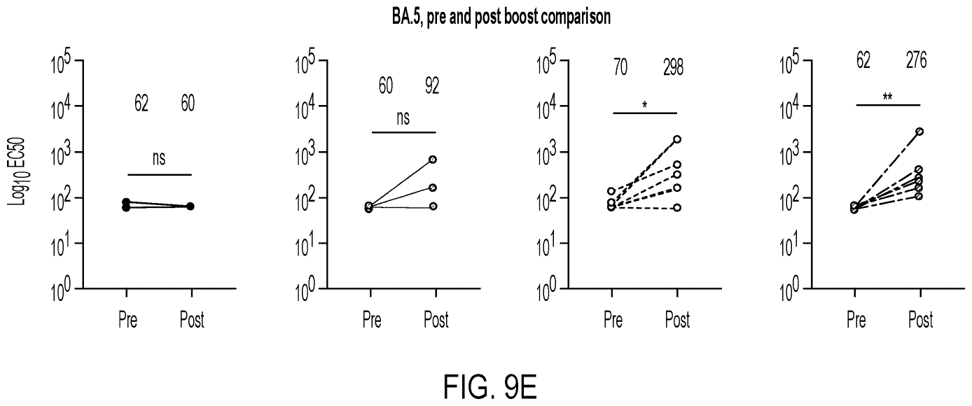

To evaluate the effects of a booster dose of mRNA-1273 (SEQ ID NO: 1 [ORF SEQ ID NO: 3, encoding SEQ ID NO: 5]), mRNA-1273.222 (SEQ ID NO: 1 and SEQ ID NO: 9 [ORF SEQ ID NO: 10, encoding SEQ ID NO: 12] at a 1:1 ratio), and mRNA-1273.214 (SEQ ID NO: 1 and SEQ ID NO: 6 [ORF SEQ ID NO: 7, encoding SEQ ID NO: 8] at a 1:1 ratio) on antibody responses and protection against BA.4/BA.5, 7-week-old K18-hACE2 mice, received 2 IM injections of 0.25 μg of UNFIX-01 (control) or mRNA-1273 approximately 3 weeks apart (primary series). Approximately 31 weeks after the second dose, mice were boosted with 0.25 μg of mRNA-1273, mRNA-1273.214, mRNA-1273.222, UNFIX-01, or PBS. Four weeks after the booster dose, mice were challenged intranasally with 10⁴ FFU of BA.5. Blood samples were collected immediately before the boost dose and at 4 weeks after the booster dose (immediately before challenge), and samples were analyzed for serum neutralization antibodies (nAbs) using focus reduction neutralization test with authentic SARS-CoV-2 strains. Mice were euthanized 4 days post-infection, and tissue (nasal wash, nasal turbinates, and lung) was harvested for virological analysis. The study protocol is shown in FIG. 9A.

Pre-boost nAb titers against WA.1/2020+D614G and Delta B.1.617.2 were relatively similar, as expected considering all mice were immunized with 0.25 μg mRNA-1273 vaccine (FIGS. 9B, 9C). However, no neutralizing activity in the pre-boost serum was detected against BA.1 or BA.5 at the 1/60 limit of detection (FIGS. 9D, 9E). Four weeks after boosting with the 0.25 μg mRNA-1273 vaccine, increased serum nAb titers against WA.1/2020+D614G and Delta B.1.617.2 were observed compared with the pre-boost titers (FIGS. 9B. 9C). Small increases in nAb titers against BA.1 and BA.5 were also observed; however, no noticeable boost effect was elicited in most mice (FIGS. 9D, 9E). In comparison, boosting with either 0.25 μg mRNA-1273.214 vaccine or 0.25 μg mRNA-1273.222 vaccine produced generally similar boost effects, with slight variations. The WA.1/2020+D614G nAb titers after boosting with the mRNA-1273.222 vaccine increased 6-fold compared with a 4-fold increase elicited by boosting with the mRNA-1273.214 vaccine (FIG. 9B). The post-boost response elicited by both mRNA-1273.222 and mRNA-1273.214 vaccines against Delta B.1.617.2 was similar (FIG. 9C). As expected, serum nAb titers against BA.1 increased by approximately 7-fold after boosting with the mRNA-1273.214 vaccine, whereas the BA.1 nAb titers increased by approximately 3-fold after boosting with the mRNA-1273.222 vaccine (FIG. 9D). Boost effect elicited by the mRNA-1273.222 and mRNA-1273.214 vaccines against BA.5 was similar, with serum BA.5 nAb titers increased by approximately 4.5- and 4.3-fold from the pre-boost levels, respectively (FIG. 9E).

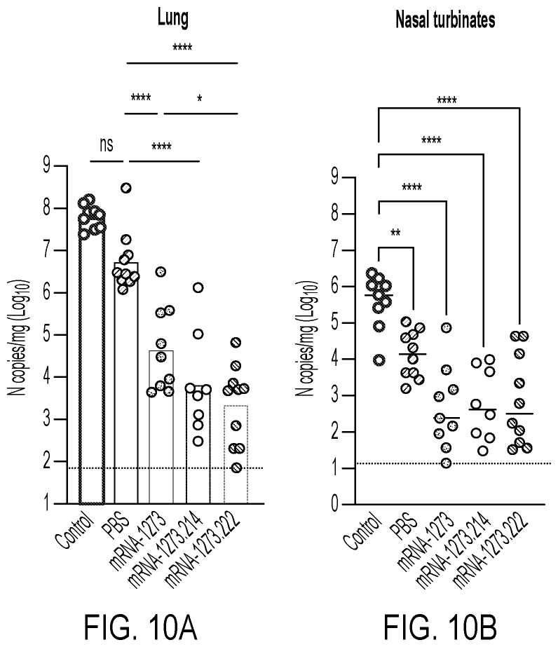

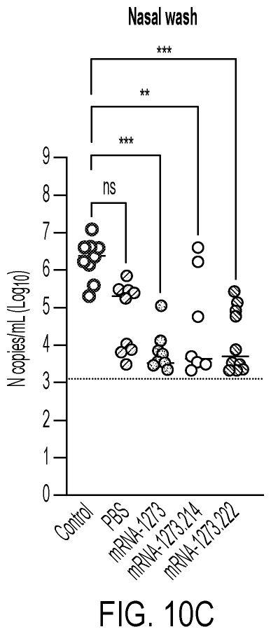

Four weeks after boosting with 0.25 μg mRNA-1273, mRNA-1273.214, mRNA-1273.222, or control (PBS) vaccines, mice were challenged with BA.5, and viral load was measured in the upper and lower respiratory tract (FIGS. 10A-10C). In mice vaccinated with 0.25 μg of mRNA-1273 (primary series), and boosted with the mRNA-1273 vaccine, viral load in lungs, nasal turbinates, and nasal wash was substantially lower than in mice boosted with PBS only or mice primed and boosted with UNFIX-01 control. Compared with PBS control, BA.5 viral load in mice vaccinated with 0.25 μg of mRNA-1273 (primary series) and boosted with the mRNA-1273 vaccine was lower in lungs and nasal turbinates and was similar in nasal wash. Protection following booster dosing with mRNA-1273.214 or mRNA-1273.222 vaccine was comparable to that observed following boosting with the mRNA-1273 vaccine, as evident from the generally similar BA.5 viral load observed in nasal turbinates and nasal wash. Boosting with the mRNA-1273.214 or mRNA-1273.222 vaccines produced a substantially lower viral load in lungs, nasal turbinates, and nasal wash compared to the control (UNFIX-01 or PBS). In terms of trends in the lungs, the degree of protection against BA.5 infection elicited after boosting with the mRNA-1273.222 vaccine was the highest followed by that elicited by the mRNA-1273.214 vaccine and then by the mRNA-1273 vaccine, with the level of protection elicited by mRNA-1273.222 reaching statistical significance versus mRNA-1273. Overall, based on evaluation of viral load after boosting with the mRNA-1273.214 or mRNA-1273.222 vaccine, both bivalent vaccines improved nAb response and reduced BA.5 viral load in the upper and lower respiratory tract.

### BALB/c Mice

Seventeen-week-old female BALB/c mice (n=8/group) received 2 intramuscular injections of PBS control article or 1 μg mRNA vaccines as a primary series 3 weeks apart. Blood was collected from all animals on Day 21 (before second dose administered) and Day 35 (2 weeks after second dose). Serum samples were analyzed for bAb responses via ELISA and nAb responses via VSV-based and lentivirus-based PSVNAs. Due to the lack of a standard SARS-CoV-2 neutralization assay, both lentivirus-based and VSV-based PSVNAs were used in this study to comprehensively capture the nAb response and to maximize throughput.

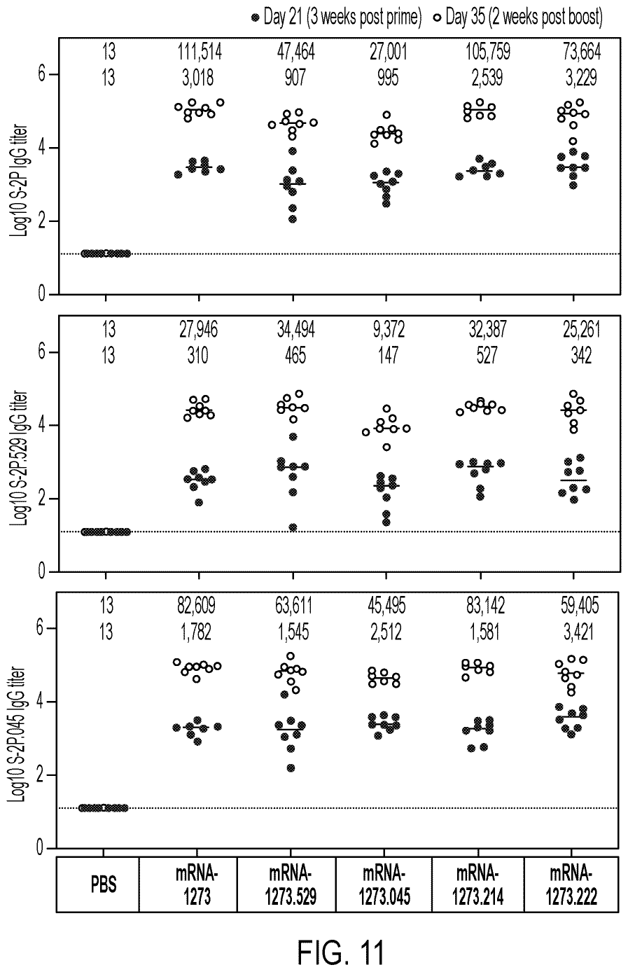

Robust binding antibody (IgG) titers against spike protein with 2 proline substitutions within the heptad repeat 1 domain (S-2P), BA.4/BA.5 Omicron-specific S-2P (S-2P.529), and BA.1 Omicron-specific S-2P (S-2P.045) proteins were observed after a 2-dose primary series with monovalent mRNA-1273, mRNA-1273.529, and mRNA-1273.045 vaccines and bivalent mRNA-1273.214 and mRNA-1273.222 vaccines, compared with control (PBS) (FIG. 11).

On Day 21 (3 weeks after the first dose), S-2P IgG GMT values ranged from 907 to 3229 and increased by approximately 23- to 52-fold on Day 35 (2 weeks after the second dose), with GMT values ranging from 27001 to 111514 across the treatment groups (FIG. 11, top graph). On Day 35, the mice vaccinated with monovalent mRNA-1273, bivalent mRNA-1273.214, or mRNA-1273.222 achieved higher S-2P IgG GMTs than mice vaccinated with monovalent mRNA-1273.529 or mRNA-1273.045.

On Day 21 (3 weeks after the first dose), S-2P.529 IgG GMT values ranged from 147 to 527 and increased by approximately 61- to 90-fold on Day 35 (2 weeks after the second dose), with GMT values ranging from 9372 to 34494 across the treatment groups (FIG. 11, middle graph). There were no notable differences in S-2P.529 IgG GMTs across treatment groups on Day 35, except mice vaccinated with monovalent mRNA-1273.045 had lower S-2P.529 IgG GMTs when compared with other treatment groups.

On Day 21 (3 weeks after the first dose), S-2P.045 IgG GMT values ranged from 1545 to 3421 and increased by approximately 17- to 52-fold on Day 35 (2 weeks after the second dose), with GMT values ranging from 45495 to 83142 across mRNA groups (FIG. 11, bottom graph). On Day 35, robust S-2P.045 IgG GMTs were observed in all mRNA groups and no notable difference was observed.

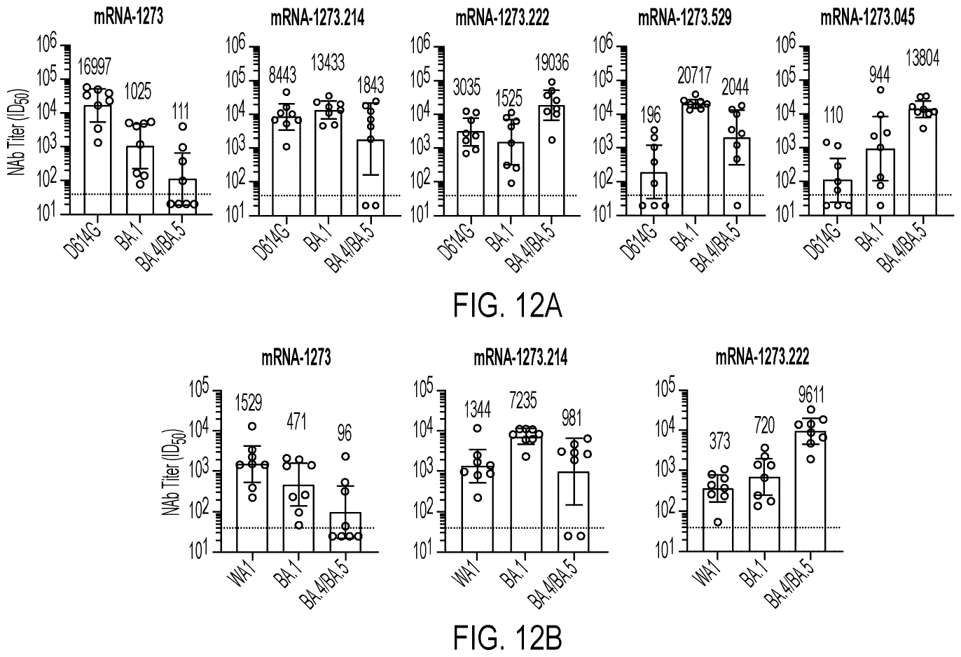

Based on the VSV-based PSVNA (FIG. 12A), on Day 35 (2 weeks after the second dose), robust nAb response against BA.4/BA.5 was observed in mice vaccinated with the bivalent mRNA-273.222 (19036) or monovalent mRNA-1273.045 (13804). When compared with the nAb response against WA.1+D614G elicited by the monovalent mRNA-1273 vaccine (16997), the nAb response against BA.4/BA.5 titers elicited by the bivalent mRNA-1273.222 vaccine (19036) was numerically higher, but similar. Furthermore, the nAb response against WA.1+D614G elicited by the bivalent mRNA-1273.222 vaccine was higher compared to that elicited by the monovalent mRNA-1273.045 vaccine (3035 versus 110, respectively). The bivalent mRNA-1273.214 and monovalent mRNA-1273.529 vaccines showed robust neutralization against BA.1, as expected, with a slightly higher GMT elicited by the mRNA-1273.529 vaccine (13433 and 20717, respectively). However, the bivalent mRNA-1273.214 vaccine conferred much higher neutralization against WA.1+D614G (8443) compared to the monovalent mRNA-1273.529 vaccine (196). The monovalent mRNA-1273 vaccine showed a robust response against WA.1+D614G (16997), but a lower response against BA.1 (1025) and BA.4/BA.5 (111).

Results of nAb response assessed using the lentivirus-based PSVNA against WA.1, BA.1, and BA.4/BA.5 following 2-dose primary vaccination series of the monovalent mRNA-1273 and bivalent mRNA-1273.214 and mRNA-1273.222 vaccines trended similarly to those observed using the VSV-based PSVNA. On Day 35 (2 weeks after the second dose), mice vaccinated with the bivalent mRNA-1273.222 had a robust nAb response against BA.4/BA.5, while the response against WA.1 and BA.1 was lower (FIG. 12B). The monovalent mRNA-1273 vaccine showed robust neutralization against WA.1 but response against BA.1 and BA.4/BA.5 was lower than that seen against WA.1. The bivalent mRNA-1273.214 vaccine showed robust nAb response against BA.1 and a lesser response against WA.1 and BA.4/BA.5.

Overall, both the bivalent mRNA-1273.222 and mRNA-1273.214 vaccines offered the best neutralization breadth.

### Example 3. Phase 2/3 Study to Evaluate the Immunogenicity and Safety of mRNA Vaccine Boosters for SARS-CoV-2 Variants

This was an open-label, Phase 2/3 study designed to evaluate the immunogenicity, safety, and reactogenicity of mRNA-1273 (SEQ ID NO: 1 [ORF SEQ ID NO: 3, encoding SEQ ID NO: 5]) and mRNA-1273.222 (SEQ ID NO: 1 and SEQ ID NO: 9 [ORF SEQ ID NO: 10, encoding SEQ ID NO: 12] at a 1:1 ratio) formulated in lipid nanoparticles and administered as second booster doses. The lipid nanoparticles comprised Compound 1, cholesterol, DSPC, and PEG-2000 DMG. Each vaccine was formulated as a sterile liquid for injection at a concentration of 0.1 mg/mL in 20 mM Tris buffer containing 87 mg/mL sucrose and 2.1 mM acetate at pH 7.5. Subjects (adults, at least 18 years of age) were administered the vaccine candidate intramuscularly as a second booster dose to subjects who have previously received 2 doses of 100 μg mRNA-1273 as a primary series and a single booster dose of 50 μg mRNA-1273. On Day 1, the candidate vaccines were administered as a single dose. The immunogenicity of the mRNA 1273.222 candidate administered as the second booster dose was compared to the immunogenicity of the second booster dose of mRNA 1273 (50 μg).

### Subject Population

Participants (males and non-pregnant females 18 years of age or older at time of consent) were included in the study if they were in good health according to the assessment of the investigator, met all eligibility criteria, and could comply with study procedures.

TABLE 2
Treatment Cohorts

| Vaccine | | Median Years | Median Days Since |
|---|---|---|---|
| Candidate | Sample Size | of Age | First Booster |
| mRNA-1273 | 376 | 60.5 (20-96) | 289 (258-312) |
| mRNA-1273.222 | 511 | 50 (19-89) | 134 (118-150) |

### Objectives

The primary objectives were to demonstrate the non-inferiority of the antibody response of a second booster dose of mRNA-1273.222 (50 μg) compared to mRNA-1273 (50 μg) when administered as a second booster dose against BA.4/5 based on the GMT ratio and seroresponse rate (SRR) at Day 29 and to demonstrate superiority of the antibody response of a second booster of mRNA-1273.222 compared to mRNA-1273 administered as a second booster dose against the Omicron BA.4/5 based on the GMT ratio at day 29. The secondary objective was to evaluate the immunogenicity of mRNA-1273.222 (50 μg) as a second booster dose against the ancestral SARS-CoV-2 (and other variants) compared to a second booster dose of mRNA-1273 (50 μg) at all timepoints post-booster. This objective was measured by examining the GMT ratio of the two vaccines against ancestral SARS-CoV-2 (and other variants) at all timepoints post-booster and the SRR difference between the two vaccines against ancestral SARS-CoV-2 and variants of concern at all timepoints post-booster. An exploratory objective was to assess symptomatic and asymptomatic SARS-CoV-2 injection by looking at laboratory-confirmed diagnoses. A second exploratory endpoint was the evaluation of genetic and/or phenotypic relationships of isolated SARS-CoV-2 strains to the vaccine sequence by characterizing the SARS-CoV-2 genomic sequence of viral isolates and comparing that with the vaccine sequence and by characterizing immune response to vaccine breakthrough isolates.

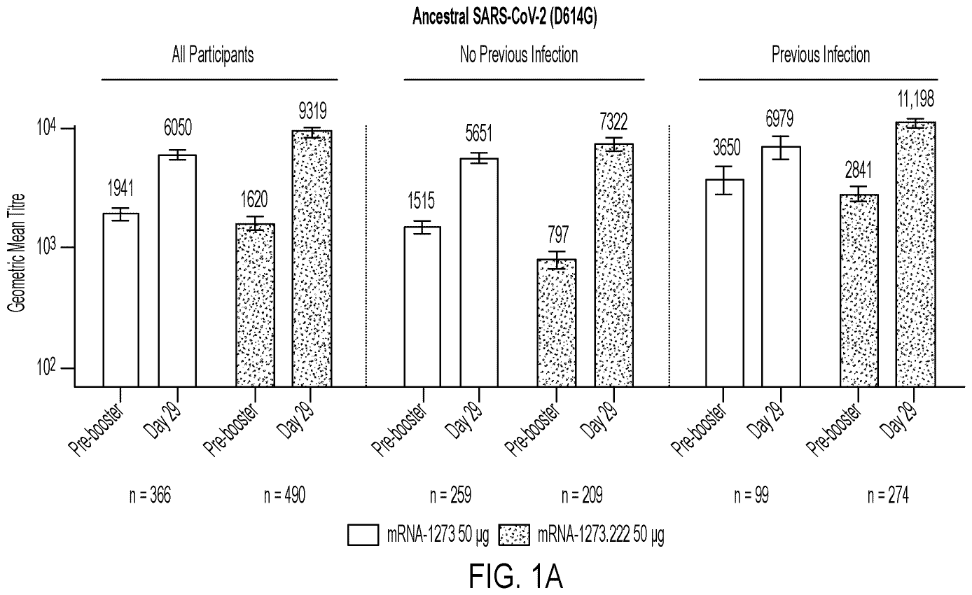

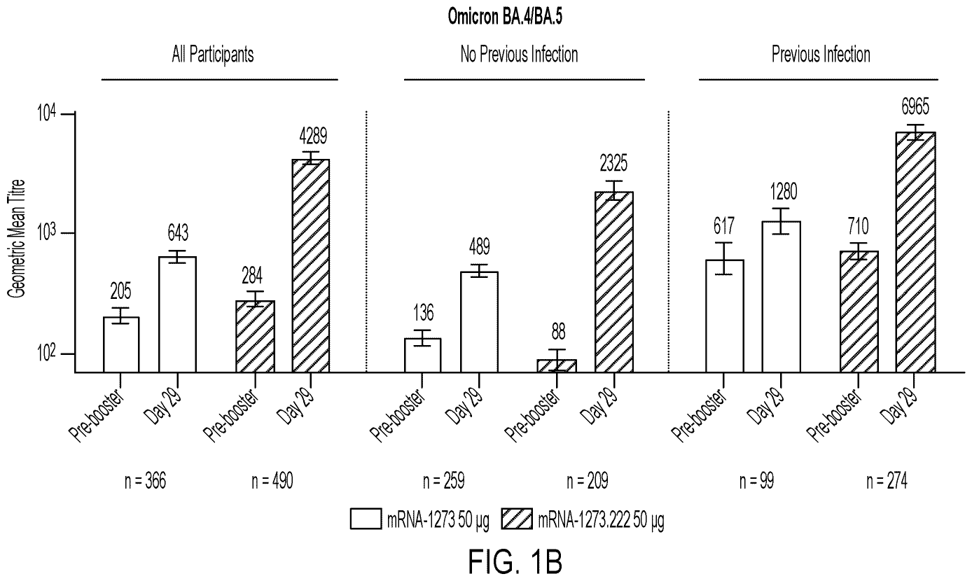

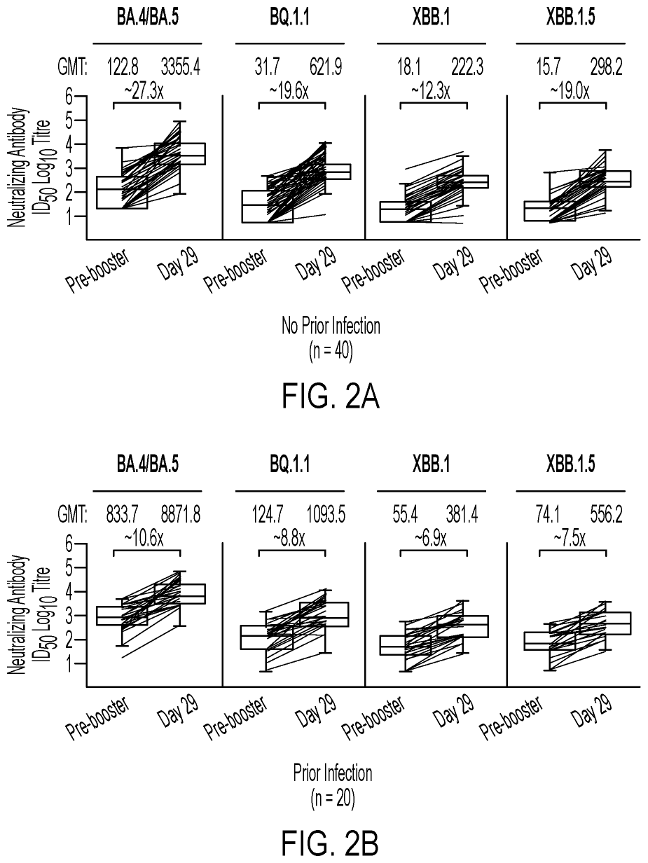

Results are shown in FIGS. 1A, 1B, 2A, and 2B. FIGS. 1A-1B show the GMT of subjects against ancestral SARS-CoV-2 (614G) (FIG. A) or Omicron BA.4/BA.5 (FIG. 13), with or without previous SARS-CoV-2 infection, before and after receiving a second booster of vaccine candidate mRNA-1273.222 and mRNA-1273. Increases in the GMT of nAb against both SARS-CoV-2 and Omicron BA.4/BA.5 were observed for both vaccine candidates, regardless of previous infection status. Also, the omicron BA.4/5-containing booster dose elicited higher nAb responses as compared to the original vaccine. The results are presented in Table 3 below.

TABLE 3
GMT and SRR for Ancestral SARS-CoV-2 (D614G) in
Participants with No Prior SARS-CoV-2 Infection

| | Ancestral SARS-CoV-2 (D614G) | | Omicron BA.4/BA.5 | |
|---|---|---|---|---|
| | mRNA-1273.222 | mRNA-1273 | mRNA-1273.222 | mRNA-1273 |
| | (n = 209) | (n = 259) | n = 209 | n = 259 |
| Pre-booster, n | 209 | 259 | 209 | 259 |
| Observed GMT | 796.9 | 1515.4 | 87.9 | 136.1 |
| (95% CI) | (678.7-935.8) | (1347.5-1704.2) | (72.2-107.1) | (116.3-159.3) |
| Day 29, n | 209 | 259 | 209 | 259 |
| Observed GMT | 7322.4 | 5651.4 | 2324.6 | 488.5 |
| (95% CI) | (6386.2-8395.7) | (5055.7-6317.3) | (1921.2-2812.7) | (427.4-558.4) |
| Estimated GMT | 9555.8 | 4882.2 | 2747.3 | 436.7 |
| (95% CI) | (8593.6-10625.7) | (4457.7-5347.1) | (2399.2-3145.9) | (389.1-490.0) |
| GMR | 1.96 | | 6.29 | |
| (95% CI) | (1.70-2.25) | | (5.27-7.51) | |
| Day 29 SRR, n/N | 209/209 | 259/259 | 205/209 | 222/257 |
| (%) | (100) | (100) | (98.1) | (86.4) |
| (95% CI) | (98.3-100.0) | (98.6-100.0) | (95.2-99.5) | (81.6-90.3) |

| | | |
|---|---|---|
| Difference | 0 | 12.1 |
| (95% CI) | | (6.9-17.3) |

Prespecified non-inferiority and superiority criteria were met in subjects with no prior SARS-CoV-2 infection. In addition, both vaccine candidates were effective in soliciting increases in nAb against ancestral SARS-CoV-2 in previously uninfected subjects, as measured by GMT and SRR. However, mRNA-1273.222 was demonstrably superior to mRNA-1273 in the same population, soliciting a heightened nAb response to Omicron BA.4/BA.5 variants. Overall, these findings indicate that mRNA-1273.222 is non-inferior against ancestral SARS-CoV-2 and superior against BA.4/BA.5 when compared to mRNA-12733 alone.

FIGS. 2A and 2B show nAb titers against different variants (Omicron BA.4/BA.5, BQ.1.1, XBB.1, and XBB.1.5) as measured by IgG ELISA. Titers were calculated at baseline (Pre-booster) and Day 29. nAb titer was presented with its corresponding 95% confidence interval (CI) estimates, GMT, and fold change at each timepoint for each variant, for subjects with and without prior infection (FIGS. 2A-2B). Neutralizing antibodies against all variants were increased on Day 29 for vaccinated subjects, regardless of infection history, possibly indicative of broadly protective effects of mRNA-1273. Notably, mRNA-1273.222 increased nAb responses to other variants (e.g., BQ.1.1, XBB.1, and XBB.1.5) compared to pre-booster titers against other variants not contained in the vaccine.

In addition, laboratory-confirmed infections by SARS-CoV-2 were reported in 3.3% of all participants (regardless of previous infection status) who received mRNA-1273.222. Asymptomatic infections were identified in 1.8% of all subjects (52.9% of infected participants), and the remaining infected subjects (47.1% of infected participants) experienced COVID-19 events, as defined by the CDC. No emergency department visits or hospitalizations occurred as a result of these events. These findings suggest a protective role for mRNA-1273.222 in patients infected by SARS-CoV-2 after vaccination.

### Example 4. Immunostimulatory/Immunodynamic Model of mRNA-1273 to Guide Pediatric Vaccine Dose Selection

The coronavirus disease 2019 (COVID-19) pandemic caused by the severe acute respiratory syndrome coronavirus 2 (SARS-CoV-2) has resulted in >6 million deaths globally (The World Health Organization. WHO Coronavirus (COVID-19) Dashboard. covid19.who.int/). Since its identification in 2019 (Centers for Disease Control and Prevention. Basics of COVID-19. cdc.gov/coronavirus/2019-ncov/your-health/about-covid-19/basics-covid-19.html (2021)), several vaccines against COVID-19 have been rapidly developed and deployed. Among those approved in the United States is the mRNA-based vaccine, mRNA-1273 (COVID-19 Vaccine, SPIKEVAX; Moderna Inc., Cambridge, MA, USA), a two-dose primary series vaccine authorized for individuals >6 months of age (Centers for Disease Control and Prevention. Stay Up to Date with Your COVID-19 Vaccines. cdc.gov/coronavirus/2019-ncov/covid-data/covidview/past-reports/082622.html (2022)).

The mRNA-based vaccine platform offers several advantages for vaccine development because it can be easily adapted and allows for scalable manufacturing (John, S., et al. “Multi-antigenic human cytomegalovirus mRNA vaccines that elicit potent humoral and cell-mediated immunity.” Vaccine 36.12 (2018):1689-1699; Pardi, N., Hogan, M. J., Porter, F. W. & Weissman, D. mRNA vaccines—a new era in vaccinology. Nature reviews drug discovery 17.4 (2018): 261-279). Conventionally, determining the optimal dose for registrational trials relies on clinical safety and efficacy data obtained from phase 1 and 2 clinical trials. However, despite being used extensively in therapeutic dose selection, mathematical modeling and pharmacometrics have not yet been commonly employed in vaccine development. Through this work, it is demonstrated that pharmacometric modeling and simulation approaches can be employed to integrate immunological principles, and to target biological and vaccine candidate-specific responses to guide clinical dose selection for vaccines in future studies. This approach enables informed predictions of vaccine responses at various stages of development and thus can significantly enhance vaccine development overall.

Immunostimulatory/immunodynamic (IS/ID) models are semi-mechanistic mathematical models that describe the immune response stimulated by vaccine exposure (IS) and the resulting antibody response dynamics (ID). Analogous to pharmacokinetic/pharmacodynamic models, IS/ID models can be used as a practical tool to help guide dose selection for clinical vaccine studies and accelerate vaccine development (Rhodes, S. J., et al. “Using vaccine Immunostimulation/Immunodynamic modelling methods to inform vaccine dose decision-making.” NPJ vaccines 3.1 (2018): 36). Previous studies have used models representing the immune response to infection and vaccination that may be considered IS/ID models (Le, D., Miller, J. D. & Ganusov, V. V. “Mathematical modeling provides kinetic details of the human immune response to vaccination.” Frontiers in cell and infection microbiology 4 (2015): 177; Chen, X., Hickling, T. P. & Vicini, P. “A mechanistic, multiscale mathematical model of immunogenicity for therapeutic proteins: part 1-theoretical model.” CPT Pharmacometrics & systems pharmacology 3.9 (2014): e133); however, to date, such models have not been used to inform vaccine dose prediction during clinical development (Rhodes, S. J., et al. “Using vaccine Immunostimulation/Immunodynamic modelling methods to inform vaccine dose decision-making.” NPJ vaccines 3.1 (2018): 36). This report describes the development of an IS/ID model used to quantitatively capture the antibody titers elicited by mRNA-1273 (10-, 25-, 50-, and 100-μg dose levels) and the utility of this model to guide optimal vaccine dose selection in pediatric clinical trials.

### Methods

### Clinical Data.

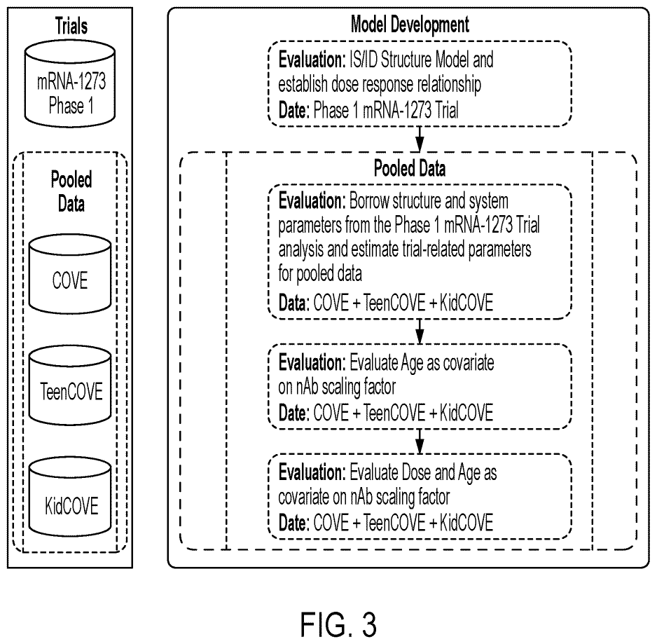

Log 10 transformed pseudovirus neutralizing antibody (nAb) ID50 titer (Log PSVN50) data were obtained from multiple phase 1 studies and two phase 2/3 and one phase 3 clinical study (TeenCOVE, NCT04649151; KidCOVE, NCT04796896; and COVE, NCT04470427; FIG. 3 and Table 4) (Ali, K., et al. “Evaluation of mRNA-1273 SARS-CoV-2 vaccine in adolescents.” New England journal of medicine 385.24 (2021): 2241-2251; Creech, C. B., et al. “Evaluation of mRNA-1273 Covid-19 vaccine in children 6 to 11 years of age.” New England journal of medicine 386.21 (2022): 2011-2023; Baden, L. R, et al. “Efficacy and safety of the mRNA-1273 SARS-CoV-2 vaccine.” New England journal of medicine 384 (2021), 403-416; Anderson, E. J., et al. “Evaluation of mRNA-1273 vaccine in children 6 months to 5 years of age.” New England journal of medicine 387.18 (2022): 1673-1687).

TABLE 4
Distribution of participants, across studies, dose, and age groups used in IS/ID analysis.

| | | Clinical study | | | | | |
|---|---|---|---|---|---|---|---|
| | | TeenCOVE | KidCOVE | | | COVE | |
| | | (n = 379) | (n = 307) | | | (n = 1126) | |
| Dose | Overall | ≥12 y & <18 y | ≥2 y & <6 y | ≥6 m & <2 y | ≥6 y & <12 y | ≥18 y & <65 y | ≥65 y |
| (μg) | (n = 1812) | (n = 379) | (n = 66) | (n = 102) | (n = 139) | (n = 758) | (n = 368) |
| 100 | 1567 | 379 | 0 | 0 | 62 | 758 | 368 |
| | (86.5%) | (100.0%) | (0.0%) | (0.0%) | (44.6%) | (100.0%) | (100.0%) |
| 50 | 143 | 0 | 66 | 0 | 77 | 0 | 0 |
| | (7.9%) | (0.0%) | (100.0%) | (0.0%) | (55.4%) | (0.0%) | (0.0%) |
| 25 | 102 | 0 | 0 | 102 | 0 | 0 | 0 |
| | (5.6%) | (0.0%) | (0.0%) | (100.0%) | (0.0%) | (0.0%) | (0.0%) |

IS/ID, immunostimulatory/immunodynamic; m, months; y, years.

For the initial parameterization of the model, the mean of each dose level was used at each time point. For the phase 2/3 and 3 studies, all per protocol participants with an available dosing history of primary series and ≥1 measurable nAb titer were included in the analysis dataset for individual participants. In the source datasets, missing nAb titers were not included in the modeling analysis. Similarly, if the dose amount was missing for a dose associated with a recorded nAb titer and the dose amount could not be imputed, the affected nAb titer was deleted. nAb titers reported as below the lower limit of quantification were excluded from the analysis irrespective of whether these were pre-dose or post-dose time points. Any observations that were excluded were documented, along with the reason for their exclusion. Distribution plots (i.e., Log PSVN50 time plots) were generated by age and dose cohorts.

### IS/ID Model.

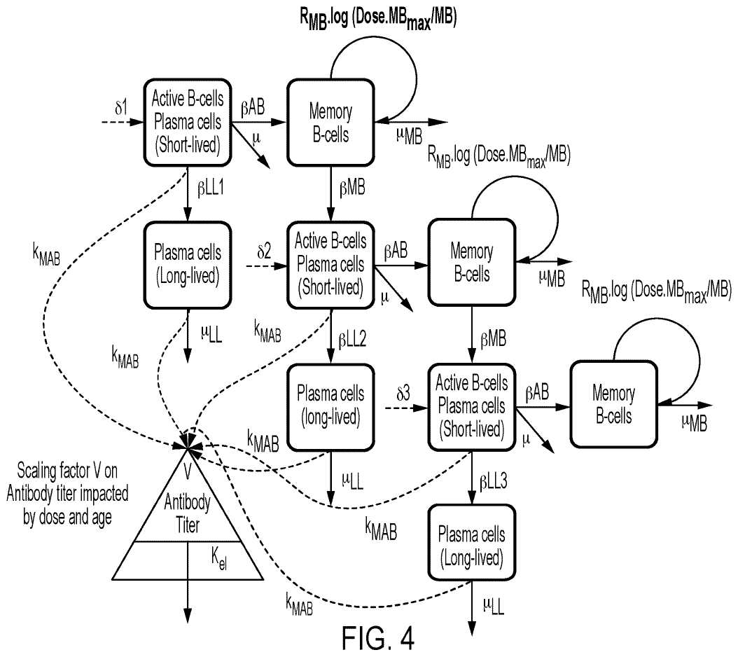

The mechanisms of immune stimulation and dynamics post-vaccination were modeled using ordinary differential equations by adapting the modeling framework used by Rhodes et al (FIG. 4) (Rhodes, S. J., et al. “Using vaccine Immunostimulation/Immunodynamic modelling methods to inform vaccine dose decision-making.” NPJ vaccines 3.1 (2018): 36). The unexplained inter-individual variability in model parameters was described using a mixed-effects model. Individual values of structural model parameters that were constrained to positive values were assumed to follow a log-normal distribution. The log-normal inter-individual variability model for structural model parameter P is given by the following equation:

$${P}_{i} = {P}_{TV} \cdot exp ( {η}_{P , i} )$$

where: Pᵢ is the value of parameter P for the ith individual; P_{TV} is the “typical value” of parameter P; and η_{P,i} is a random effect, a realization of a normally distributed random variable with 0 mean and variance ω_{P}² that describes the inter-individual variability in the parameter P.

The correlations between distributions of different structural model parameters were evaluated using the joint distribution of all random effects with a multivariate normal distribution, with mean vector 0 and variance-covariance matrix Ω. The following structures of the Ω matrix were evaluated: diagonal, where all off-diagonal elements were fixed to 0; full, where all elements of the matrix were estimated; and block-diagonal.

To explain the differences between observed values and the corresponding model predictions within an individual, a residual error model was included. A log-additive error model was employed as below, where Y is the observed nAb in log domain, μ is the model predicted nAb, and σ is the additive error:

$$log ( Y ) = Normal ( log ( μ ) , σ )$$

### Covariate Analysis.

The effects of covariates such as study, age, and dose on individual random effects were evaluated graphically. To assess whether the addition of covariate effects removed any residual trends, individual random effects versus covariates included in the final model were compared in the base and final models. Correlation plot matrices of individual random effects were explored to assess possible correlations between parameters. Individual fitted plots overlaid on observed data were examined for the final model to determine if the model was able to capture the trends in the observed data on an individual level and to identify any unexplained or potentially erroneous data.

### Stability and Predictive Performance of the Final Model.

The following criteria were considered in performing model selection: goodness-of-fit diagnostic plots and other graphical assessments, minimum value of the objective function, shrinkage of the deviation of an individual-specific parameter estimate from the population typical value, visual predictive checks, and graphical assessment.

Nonparametric bootstrap was used to quantify the uncertainty of the parameter estimates from the final model. The dataset was resampled 100 times with replacement to obtain 100 replicate datasets. Resampling was performed at the individual level so that each replicate dataset contained the same number of individuals as the original dataset. The final model was refitted to each resampled dataset, and the distribution of the parameter estimates across replicates was used to compute nonparametric 95% confidence intervals (CI).

For evaluating the predictive performance of the model, the final model was used to simulate 200 replicates of the combined dataset at a very rich time grid of every 3 days. Summary statistics of interest were determined by time point across simulation replicates. These summary statistics were compared with observed data to assess concordance between the model-based simulations and the observed data.

### Non-Inferiority Criteria.

The non-inferiority of nAb geometric mean ratios (GMRs) at day 57 in young children (aged 2-5 years) and infants (aged 6-23 months), as compared with a random subset of young adults (aged 18-25 years in a random subset of participants from the COVE study), was indicated if the lower boundary of the 95% CI for the GMR was above 0.80.

### Pediatric Dose Predictions.

Data used for fitting the model were from 98 participants aged ≥6 months to <2 years and 50 participants aged ≥2 to <6 years in the phase 2/3 KidCOVE (NCT04796896) trial (Creech, C. B., et al. “Evaluation of mRNA-1273 Covid-19 vaccine in children 6 to 11 years of age.” New England journal of medicine 386.21 (2022): 2011-2023; Anderson, E. J., et al. “Evaluation of mRNA-1273 vaccine in children 6 months to 5 years of age.” New England journal of medicine 387.18 (2022): 1673-1687). Part 1 of KidCOVE is designed to test different doses of the mRNA-1273 vaccine, and was ongoing at the time of this analysis. To gain reassurance and assess the need for dose changes in the ongoing trial, a dose projection exercise was conducted.

The optimized model based on the limited pediatric data was used to predict the day 57 geometric mean titer (GMT) in the pediatric age groups (≥6 months to <2 years, ≥2 to <6 years, ≥6 to <12 years, and ≥12 to <18 years) at doses of 10, 25, 50, and 100 μg. Subsequently, the GMR was computed using the day 57 GMT observed in participants aged 18 to 25 years.

Simulations were conducted with the final model to characterize the dose-response relationship in the pediatric age groups (≥6 months to <2 years, ≥2 to <6 years, ≥6 to <12 years, and ≥12 to <18 years). The GMR of the simulated nAb GMT was used as a metric to characterize the dose-response relationship. Specifically, the IS/ID model was used to simulate doses of 10, 25, 50, and 100 μg. The simulations were conducted using the Empirical Bayes estimates of IS/ID fit of the phase 2 and 3 data. Each of the four age cohorts (≥6 months to <2 years, ≥2 to <6 years, ≥6 to <12 years, and ≥12 to <18 years) received all four dose levels of mRNA-1273. nAb titer was simulated at day 57 for all participants. GMR was computed for each dose and age group as the ratio of the day 57 GMT to the day 57 GMT of the phase 3 COVE (NCT04470427) trial (Baden, L. R. et al. “Efficacy and safety of the mRNA-1273 SARS-CoV-2 vaccine.” New England journal of medicine 384.5 (2021): 403-416) participants aged 18 to 25 years.

### Software.

The population IS/ID model was developed using nonlinear mixed effects modeling software Pumas (version 2.0.3; Pumas-AI, Inc., Baltimore, MD, USA). Estimation was performed by first-order conditional estimation with interaction. Nonparametric bootstrap was used to quantify parameter uncertainty. Pumas and Julia language (version 1.6.2) used on JuliaHub (version 5.4.3; Julia Computing, Inc.) was employed for data preparation, graphical analysis, model diagnostics, statistical summaries, and simulation of the pediatric dose projections.

### Results

### IS/ID Model Development.

Using data from the phase 1 mRNA-1273-P101 trial (NCT04283461; FIG. 3) (Jackson, L. A., et al. “An mRNA vaccine against SARS-CoV-2—preliminary report.” New England journal of medicine 383.20 (2020): 1920-1931), an IS/ID model to describe the antibody titer dynamics was developed by adapting the modeling framework used by Rhodes et al (FIG. 4) (Rhodes, Sophie J., et al. “Using vaccine Immunostimulation/Immunodynamic modelling methods to inform vaccine dose decision-making.” Npj Vaccines 3.1 (2018): 36). Dynamics of nAb titers generated following vaccination were attributed to several populations of B cells. B-cell response is delayed following vaccination due to immune processes such as vaccine antigen trafficking and presentation (Abbas, A., Lichtman, A. & Pillai, S. Cellular and Molecular Immunology [Internet], 2015; Urdahl, K. B., S. Shafiani, and J. D. Ernst. “Initiation and regulation of T-cell responses in tuberculosis.” Mucosal immunology 4.3 (2011): 288-293) and does not last indefinitely (Urdahl, K. B., S. Shafiani, and J. D. Ernst. “Initiation and regulation of T-cell responses in tuberculosis.” Mucosal immunology 4.3 (2011): 288-293). It was assumed that the B-cell activation rate (δ) was nonlinear. δ was initiated at the time of the first and the second vaccine dose and was assumed to be the same at all vaccination points. The rate at which B cells were activated and entered active B-cell pools was assumed to follow a Gaussian equation:

$$δ = a \cdot ( {e}^{\frac{- {( t - b )}^{2}}{2 {c}^{2}}} + {e}^{\frac{- {( t - ( b + revac \_ time ) )}^{2}}{2 {c}^{2}}} ) \qquad ( 1 )$$

Here, a scales the shape δ; an increase in a is expected to increase the magnitude of the activated B-cell population and therefore the overall response. a does not regulate the timing of the recruitment, only the absolute number of activated B cells. b is the Gaussian equation mean and affects the activated B-cell recruitment time. c is the variance in the Gaussian equation that describes δ and acts on the overall magnitude of response. t is time, measured in days, and revac_time is revaccination time, measured in days. The effect of mRNA-1273 dose on δ was best described by a linear model. The Gaussian equation time of peak (b) was fixed to 5 days, based on observation of the available data.

The structural model included activation of B cells following the first vaccine dose. The activated B cells transition into memory B cells and differentiate into long-lived plasma cells (LLPCs). The first vaccine dose has an impact only on the formation of memory B cells. Upon revaccination due to the second dose, in addition to the primary response, a recall response driven by previously created memory B cells with high-affinity receptors is initiated. The primed memory B cells proliferate and differentiate into antibody-secreting plasma cells. A fraction of memory B cells move back into the germinal center for further maturity affinity training; this is known as the anamnestic response (Weisel, F. J., et al. “Unique requirements for reactivation of virus-specific memory B lymphocytes.” The Journal of Immunology 185 (2010): 4011-4021). As the memory B cells are primed to the antigen during the first vaccine dose, the response is now rapid due to a lower activation threshold. Thus, memory B cells quickly generate antibody-secreting plasma cells of higher quality, leading to a greater amplitude of secreted antibodies (Ademokun, Alexander A., and Deborah Dunn-Walters. “Immune responses: primary and secondary.” eLS (2010); Tangye, Stuart G., et al. “Intrinsic differences in the proliferation of naive and memory human B cells as a mechanism for enhanced secondary immune responses.” The Journal of Immunology 170.2 (2003): 686-694)).

Following revaccination (second vaccine dose), two processes were initiated simultaneously in the model: first, memory B cells replicate at a rate R_{MB} for τ days. Following replication, memory B cells differentiate to antibody-secreting plasma cells. These cells enter the “activated B-cell” population, at rate β_{MB}. The number of memory B cells that could be generated after vaccination was dependent on the vaccine dose and was limited to a maximum by controlling the proliferation rate of memory B cells (R_{MB}) using the following equations:

$${R}_{MB} ( t ) = {R}_{MB} \cdot ln ( \frac{{MB}_{max}}{MB ( t )} )$$

$${MB}_{max} = Dose \cdot SLOPE$$

where: R_{MB}(t) is the proliferation rate of memory B cells at time t, MBₘₐₓ is the maximum number of memory B cells, MB(t) is the number of memory B cells at time t, Dose is mRNA-1273 dose in μg; and SLOPE is the number of memory B cells that are generated per each 1-μg dose of mRNA-1273. Thus, the second vaccine dose has an impact on both formation and proliferation (R_{MB}) of memory B cells. As with primary vaccination, B cells were again activated and entered the activated B-cell compartment at rate δ.

The observed antibody titer was assumed to be produced by short-lived plasma cells (SLPCs) and LLPCs at rate k_{MAB}. The transition rate from active B cells to memory B cells (β_{AB}), the death rate of LLPCs (μ_{LL}), the proliferation rate of memory B cells (R_{MB}), the death rate of memory B cells (μ_{MB}), the memory B-cell replication time (τ), the secretion rate of antibody by plasma cells (k_{MAB}), and the elimination rate for antibodies (kₑₗ) were fixed to values found in the literature (Chen, X., Hickling, T. P. & Vicini, P. “A mechanistic, multiscale mathematical model of immunogenicity for therapeutic proteins: part 1-theoretical model.” CPT Pharmacometrics & systems pharmacology 3.9 (2014): e133). All other parameters were free to be estimated. The volume scaling factor (V) was used to link the number of antibodies in the body to the measured antibody titer. The expectation is that this parameter will be a function of dose and age because the measured titers will be a function of the amount of vaccine in the body and of the age at which the vaccine was received. Clinically, most vaccines are dosed based on age, and hence, we used V with this parameter (Centers for Disease Control and Prevention. Vaccines and Preventable Diseases: Recommended Vaccines by Age. cdc.gov/vaccines/vpd/vaccines-age.html (2023)).

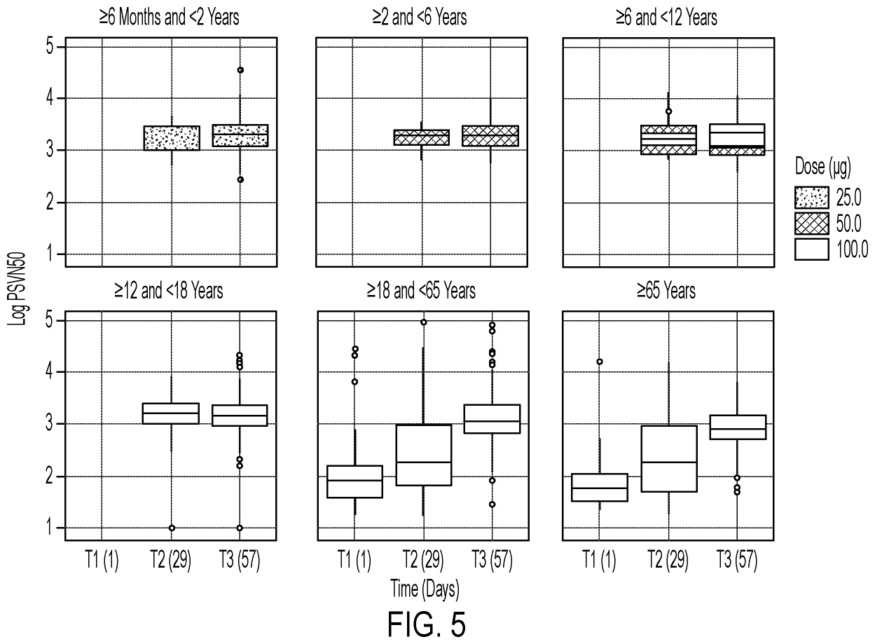

nAb responses (GMT) to mRNA-1273 by dose and age group at each available time point (days 29 and 57 for participants aged ≥18 years; days 1, 29, and 57 for participants aged <18 years) are shown in FIG. 5. In adult participants, nAb titers increased from day 29 to day 57. On average, the day 57 titers in adults matched the day 57 titers observed with lower doses in other age groups.

### Fitting the IS/ID Model to Clinical Data and Parametrization.

The IS/ID model was used to fit data from the phase 1 mRNA-1273 trial (NCT04283461) (Jackson, L. A., et al. “An mRNA vaccine against SARS-CoV-2—preliminary report.” New England journal of medicine 383.20 (2020): 1920-1931). Basic goodness-of-fit plots from this stage of the analysis indicated the structural model was able to capture both the magnitude of antibody response to antigen and the durability of the response when data was collected up to 6 months in certain patients. The results and structure of the model developed using the phase 1 mRNA-1273 data were then borrowed to estimate the final model parameters (FIG. 3, Table 5) for the pooled data from phases 2 and 3.

TABLE 5
Population IS/ID model parameters describing mRNA-1273 phase 2 and 3 data.

| Parameter | Description | Estimate | 95% CI |
|---|---|---|---|
| tva^{#} | Gaussian equation curve multiplier (number of cells) | 8.4569 | [7.401, 9.230] |
| tvb* | Gaussian equation time of peak (days) | 5.0 | — |
| tvc^{#} | Gaussian equation variance (days) | 3.9045 | — |
| tvβAB* | Transition rate to memory cells (day⁻¹) | 0.05 | — |
| tvμ | Death rate of activated B cells and SLPCs (day⁻¹) | 0.068974 | [0.036196, 0.069431] |
| tvβMB | Activation rate of memory B cells (day⁻¹) | 0.053657 | [0.043517, 0.07406] |
| tvRcm* | Proliferation rate of memory B cells (day⁻¹) | 0.727 | — |
| tvμMB* | Death rate of memory B cells (day⁻¹) | 7.83e−5 | — |
| tvτ* | Replication time of memory B-cells (days) | 5.0 | — |
| tvKmab* | Secretion rate of antibody by plasma cells (day⁻¹) | 8.64e8 | — |
| tvKel* | Elimination rate for antibody (day⁻¹) | 0.033 | — |
| tvV | Scaling factor | 6.1672e10 | [6.0561e10, 7.9371e10] |
| tvSLOPE | Number of memory B cells generated per each 1-μg | 1489.7 | [1260.0, 1621.6] |
| | dose of mRNA-1273 (cells/μg) | | |
| tvβLL1^{#} | Transition rate to LLPCs after first vaccination (day⁻¹) | 0.00044661 | — |
| tvβLL2^{#} | Transition rate to LLPCs after second vaccination | 0.0031606 | — |
| | (day⁻¹) | | |
| tvμLL* | Death rate of LLPCs (day⁻¹) | 0.005 | — |
| tvAGEonV | Age factor on V | 1.1404e9 | [8.4006e8, 1.1611e9] |
| tvDoseonV | Dose factor on V | 0.84875 | [0.83147, 1.0781] |
| Ω_{1, 1} | Between-subject variability on scaling factor | 0.447 | [0.37391, 0.53352] |
| σ₁ | Additive error on log scale | 0.31461 | [0.29632, 0.33066] |
| tvθ₁* | Proportion of antibody secretion from SLPCs | 0.55 | — |
| tvθ₂* | Proportion of antibody secretion from LLPCs | 0.44 | — |

CI, confidence interval; IS/ID, immunostimulatory/immunodynamic; LLPC, long-lived plasma cell; SLPC, short-lived plasma cell.

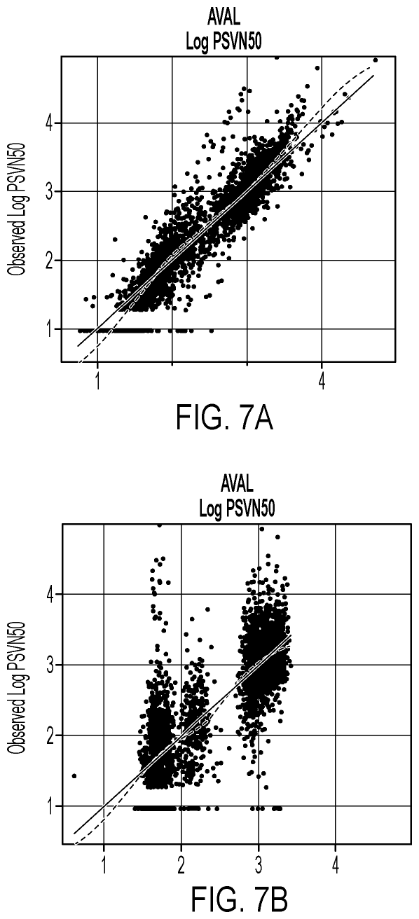

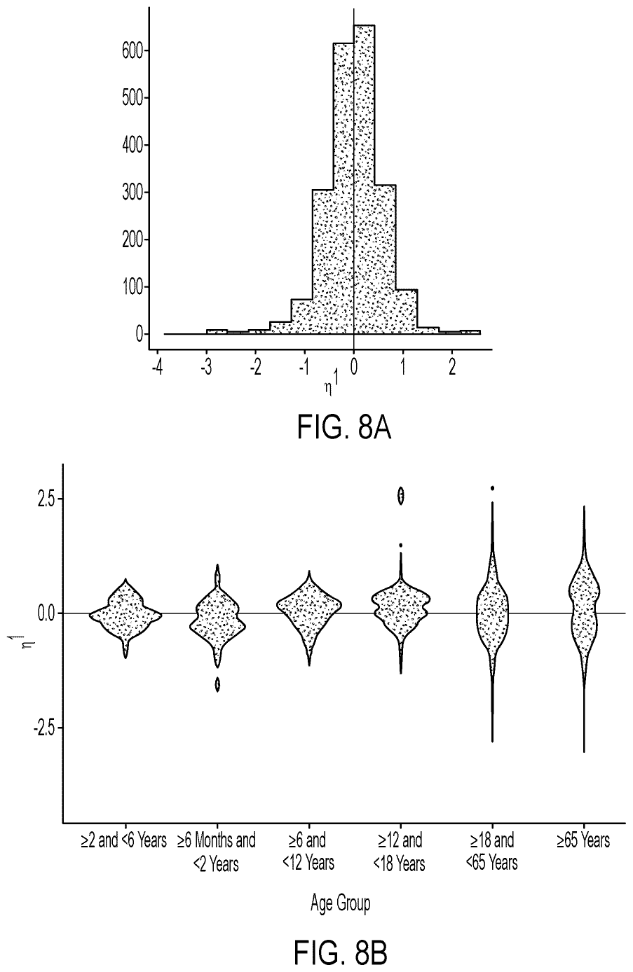

At this stage of the analysis, using the pooled data, three models were evaluated. These include the base model, a model with age as a covariate on nAb scaling factor V. and a model with dose and age as a covariate on the nAb scaling factor V. The full parameter table for the final population IS/ID model is presented in Table 5. The bootstrap medians were close to the final estimates, suggesting minimal bias, and were relatively well centered within the bootstrap 95% CI. Shrinkage on inter-individual variability random effects (q shrinkage) was acceptable for these data at 40%. Standard goodness-of-fit diagnostic plots indicated that the IS/ID model captures the observed data well (FIG. 7A, 7B, 8A, 8B).

### Pediatric Dose Projections.

The data in this analysis included 3 dose levels: 25 μg for <2 years of age, 50 μg for 2 to 12 years of age, and 100 μg for 12 to 18 and >18 years of age. Thus, the IS/ID model was developed using clinical data pooled across different dose levels and age groups and allowed for interpolating nAb titers at dose levels not evaluated in clinical studies at specific age groups. Table 6 shows the model predicted mean GMTs across dose and age groups.

TABLE 6
Observed and model-predicted nAb GMTs by clinical
study across mRNA-1273 dose levels and age groups.

| | | | | | | % Difference |
|---|---|---|---|---|---|---|
| Clinical | Dose | Age | | Observed nAb | Predicted nAb | (observed vs |
| study | (μg) | group | N | GMT | GMT | predicted GMT) |
| TeenCOVE | 100 | ≥12 y to | 340 | 1402 (1280, 1535) | 1681 (1621, 1741) | 19.9 |
| | | <18 y | | | | |
| | 50 | ≥12 y to | — | — | 1190 (1148, 1232) | |
| | | <18 y | | | | |
| KidCOVE | 100 | ≥6 y to | 57 | 1888 (1520, 2344) | 2407 (2308, 2506) | 27.5 |
| | 50 | <12 y | 320 | 1610 (1457, 1780) | 1704 (1635, 1774) | 6.17 |
| | 25 | ≥2 y to | 50 | 1014 (846, 1215) | 1124 (1069, 1180) | 10.9 |
| | 50 | <6 y | 68 | 1847 (1602, 2130) | 1694 (1610, 1777) | −8.2 |
| | 25 | ≥6 m to | 97 | 1783 (1542, 2061) | 1503 (1389.0, 618.0) | −15.7 |
| | | <2 y | | | | |
| COVE | 100 | ≥18 y | 1055 | 1081 (1020, 1146) | | |
| | 100 | ≥18 y to | 700 | 1207 (−11,261, 293) | 1300 (1193, 1407) | 7.71 |
| | | <65 y | | | | |

GMT, geometric mean titer; IS/ID, immunostimulatory/immunodynamic; m, months; nAb, neutralizing antibody; y, years.

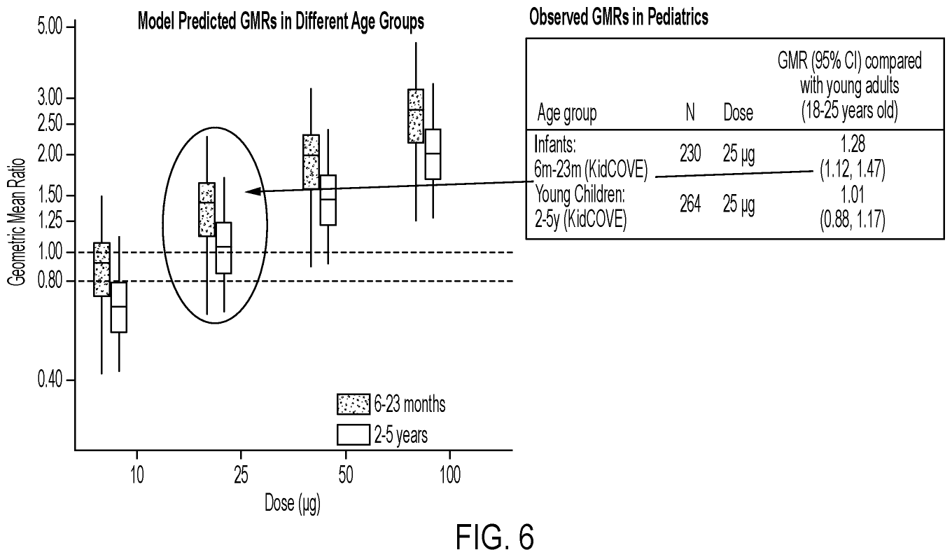

The model was used to predict the dose that met the GMT reference threshold (aged 18-25 years) for the pediatric cohort of young children (aged 2-5 years) and infants (aged 6-23 months). FIG. 6 shows the model predicted distribution of the GMRs across doses in young children (aged 2-5 years) and infants (aged 6-23 months) and shows a clear dose-response relationship within each age group. A 25-μg primary mRNA-1273 vaccine series was predicted to meet non-inferiority criteria in young children (aged 2-5 years) and infants (aged 6-23 months).

Validation of dose projections using real clinical data. The model predicted GMR of young children (aged 2-5 years) and infants (aged 6-23 months) matched well with the observed data obtained at a later point on completion of the trials (FIG. 6).

### Discussion

The study represents a first instance in which an IS/ID model was used to support vaccine clinical development and project the outcome of a pediatric dose. This modeling activity successfully predicted the immunogenic response of young children (aged 2-5 years) and infants (aged 6-23 months) to different dose levels of the COVID-19 vaccine, mRNA-1273, which was confirmed by the phase 2 pediatric KidCOVE clinical study (FIG. 6) (Creech, C. B., et al. “Evaluation of mRNA-1273 Covid-19 vaccine in children 6 to 11 years of age.” New England journal of medicine 386.21 (2022): 2011-2023; Anderson, E. J., et al. “Evaluation of mRNA-1273 vaccine in children 6 months to 5 years of age.” New England journal of medicine 387.18 (2022): 1673-1687).

The modified vaccine IS/ID modeling framework, adapted from Rhodes et al (Rhodes, S. J., et al. “Using vaccine Immunostimulation/Immunodynamic modelling methods to inform vaccine dose decision-making.” NPJ vaccines 3.1 (2018): 36), describes the immune response stimulation (IS) that produces the measured immune response dynamics following vaccination (ID) with mRNA-1273. The model has strong underpinnings of the relevant dynamics of B cells and was initially able to capture the magnitude and durability from the richer phase 1 mRNA-1273 trial data. The parameters characterizing the durability were then assumed to be the same while characterizing the magnitude of response at day 57 for the pooled phase 2 and 3 datasets. Such a model allows one to make informed dose projections characterizing the mRNA-1273 COVID-19 vaccine response and projected outcomes in untested scenarios.

As data from the phase 2/3 TeenCOVE and KidCOVE trials and the phase 3 COVE trial were generated, the IS/ID model was updated accordingly to provide support for pediatric dose selection. This validation of the IS/ID humoral response model predictions by subsequently observed data is the first of its kind.

A study strength includes the application of a semi-mechanistic mathematical model to vaccine data that are rarely explored quantitatively. A feature of this work is its direct impact on dose justification and confirmation as results accumulated over time. The model captured the essential B-cell dynamics that can be adapted toward other vaccine data.

In summary, these findings demonstrate that modeling and simulation using an IS/ID model is a novel approach that can help guide data-driven decision-making for clinical dose selection of vaccines and may accelerate vaccine development in the future.

### Additional Sequences

It should be understood that mRNA sequences may include a 5′ UTR and/or a 3′ UTR. The UTR sequences may be selected from the following sequences, or other known UTR sequences may be used. It should also be understood that any of the mRNA constructs may further comprise a poly(A) tail and/or cap (e.g., 7mG(5′)ppp(5′)NlmpNp). Further, while many of the mRNAs and encoded antigen sequences include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 13)
GGGAAAUAAGAGAGAAAAGAAGAGUAAGAAGAAAUAUAAGAGCCACC
5′ UTR:
(SEQ ID NO: 2)
GGGAAAUAAGAGAGAAAAGAAGAGUAAGAAGAAAUAUAAGACCCCGGCG
CCGCCACC
3′ UTR:
(SEQ ID NO: 14)
UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC
3′ UTR:
(SEQ ID NO: 4)
UGAUAAUAGGCUGGAGCCUCGGUGGCCUAGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC

TABLE 6
Sequence Listing
mRNA-1273

| SEQ ID NO: 1 consists of from 5′ end to 3′ end: 5′ UTR SEQ ID | | 1 |
|---|---|---|
| NO: 2, mRNA ORF SEQ ID NO: 3, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 3 |
| Construct | CAGUGCGUGAACCUGACCACCCGGACCCAGCUGCCACCA | |
| (excluding the stop | GCCUACACCAACAGCUUCACCCGGGGCGUCUACUACCCC | |
| codon) | GACAAGGUGUUCCGGAGCAGCGUCCUGCACAGCACCCAG | |
| | GACCUGUUCCUGCCCUUCUUCAGCAACGUGACCUGGUUC | |
| | CACGCCAUCCACGUGAGCGGCACCAACGGCACCAAGCGG | |
| | UUCGACAACCCCGUGCUGCCCUUCAACGACGGCGUGUAC | |
| | UUCGCCAGCACCGAGAAGAGCAACAUCAUCCGGGGCUGG | |
| | AUCUUCGGCACCACCCUGGACAGCAAGACCCAGAGCCUG | |
| | CUGAUCGUGAAUAACGCCACCAACGUGGUGAUCAAGGUG | |
| | UGCGAGUUCCAGUUCUGCAACGACCCCUUCCUGGGCGUG | |
| | UACUACCACAAGAACAACAAGAGCUGGAUGGAGAGCGAG | |
| | UUCCGGGUGUACAGCAGCGCCAACAACUGCACCUUCGAG | |
| | UACGUGAGCCAGCCCUUCCUGAUGGACCUGGAGGGCAAG | |
| | CAGGGCAACUUCAAGAACCUGCGGGAGUUCGUGUUCAAG | |
| | AACAUCGACGGCUACUUCAAGAUCUACAGCAAGCACACC | |
| | CCAAUCAACCUGGUGCGGGAUCUGCCCCAGGGCUUCUCA | |
| | GCCCUGGAGCCCCUGGUGGACCUGCCCAUCGGCAUCAAC | |
| | AUCACCCGGUUCCAGACCCUGCUGGCCCUGCACCGGAGC | |
| | UACCUGACCCCAGGCGACAGCAGCAGCGGGUGGACAGCA | |
| | GGCGCGGCUGCUUACUACGUGGGCUACCUGCAGCCCCGG | |
| | ACCUUCCUGCUGAAGUACAACGAGAACGGCACCAUCACC | |
| | GACGCCGUGGACUGCGCCCUGGACCCUCUGAGCGAGACC | |
| | AAGUGCACCCUGAAGAGCUUCACCGUGGAGAAGGGCAUC | |
| | UACCAGACCAGCAACUUCCGGGUGCAGCCCACCGAGAGC | |
| | AUCGUGCGGUUCCCCAACAUCACCAACCUGUGCCCCUUC | |
| | GGCGAGGUGUUCAACGCCACCCGGUUCGCCAGCGUGUAC | |
| | GCCUGGAACCGGAAGCGGAUCAGCAACUGCGUGGCCGAC | |
| | UACAGCGUGCUGUACAACAGCGCCAGCUUCAGCACCUUC | |
| | AAGUGCUACGGCGUGAGCCCCACCAAGCUGAACGACCUG | |
| | UGCUUCACCAACGUGUACGCCGACAGCUUCGUGAUCCGU | |
| | GGCGACGAGGUGCGGCAGAUCGCACCCGGCCAGACAGGC | |
| | AAGAUCGCCGACUACAACUACAAGCUGCCCGACGACUUC | |
| | ACCGGCUGCGUGAUCGCCUGGAACAGCAACAACCUCGAC | |
| | AGCAAGGUGGGCGGCAACUACAACUACCUGUACCGGCUG | |
| | UUCCGGAAGAGCAACCUGAAGCCCUUCGAGCGGGACAUC | |
| | AGCACCGAGAUCUACCAAGCCGGCUCCACCCCUUGCAAC | |
| | GGCGUGGAGGGCUUCAACUGCUACUUCCCUCUGCAGAGC | |
| | UACGGCUUCCAGCCCACCAACGGCGUGGGCUACCAGCCC | |
| | UACCGGGUGGUGGUGCUGAGCUUCGAGCUGCUGCACGCC | |
| | CCAGCCACCGUGUGUGGCCCCAAGAAGAGCACCAACCUG | |
| | GUGAAGAACAAGUGCGUGAACUUCAACUUCAACGGCCUU | |
| | ACCGGCACCGGCGUGCUGACCGAGAGCAACAAGAAAUUC | |
| | CUGCCCUUUCAGCAGUUCGGCCGGGACAUCGCCGACACC | |
| | ACCGACGCUGUGCGGGAUCCCCAGACCCUGGAGAUCCUG | |
| | GACAUCACCCCUUGCAGCUUCGGCGGCGUGAGCGUGAUC | |
| | ACCCCAGGCACCAACACCAGCAACCAGGUGGCCGUGCUG | |
| | UACCAGGACGUGAACUGCACCGAGGUGCCCGUGGCCAUC | |
| | CACGCCGACCAGCUGACACCCACCUGGCGGGUCUACAGC | |
| | ACCGGCAGCAACGUGUUCCAGACCCGGGCCGGUUGCCUG | |
| | AUCGGCGCCGAGCACGUGAACAACAGCUACGAGUGCGAC | |
| | AUCCCCAUCGGCGCCGGCAUCUGUGCCAGCUACCAGACC | |
| | CAGACCAAUUCACCCCGGAGGGCAAGGAGCGUGGCCAGC | |
| | CAGAGCAUCAUCGCCUACACCAUGAGCCUGGGCGCCGAG | |
| | AACAGCGUGGCCUACAGCAACAACAGCAUCGCCAUCCCC | |
| | ACCAACUUCACCAUCAGCGUGACCACCGAGAUUCUGCCC | |
| | GUGAGCAUGACCAAGACCAGCGUGGACUGCACCAUGUAC | |
| | AUCUGCGGCGACAGCACCGAGUGCAGCAACCUGCUGCUG | |
| | CAGUACGGCAGCUUCUGCACCCAGCUGAACCGGGCCCUG | |
| | ACCGGCAUCGCCGUGGAGCAGGACAAGAACACCCAGGAG | |
| | GUGUUCGCCCAGGUGAAGCAGAUCUACAAGACCCCUCCC | |
| | AUCAAGGACUUCGGCGGCUUCAACUUCAGCCAGAUCCUG | |
| | CCCGACCCCAGCAAGCCCAGCAAGCGGAGCUUCAUCGAG | |
| | GACCUGCUGUUCAACAAGGUGACCCUAGCCGACGCCGGC | |
| | UUCAUCAAGCAGUACGGCGACUGCCUCGGCGACAUAGCC | |
| | GCCCGGGACCUGAUCUGCGCCCAGAAGUUCAACGGCCUG | |
| | ACCGUGCUGCCUCCCCUGCUGACCGACGAGAUGAUCGCC | |
| | CAGUACACCAGCGCCCUGUUAGCCGGAACCAUCACCAGC | |
| | GGCUGGACUUUCGGCGCUGGAGCCGCUCUGCAGAUCCCC | |
| | UUCGCCAUGCAGAUGGCCUACCGGUUCAACGGCAUCGGC | |
| | GUGACCCAGAACGUGCUGUACGAGAACCAGAAGCUGAUC | |
| | GCCAACCAGUUCAACAGCGCCAUCGGCAAGAUCCAGGAC | |
| | AGCCUGAGCAGCACCGCUAGCGCCCUGGGCAAGCUGCAG | |
| | GACGUGGUGAACCAGAACGCCCAGGCCCUGAACACCCUG | |
| | GUGAAGCAGCUGAGCAGCAACUUCGGCGCCAUCAGCAGC | |
| | GUGCUGAACGACAUCCUGAGCCGGCUGGACCCUCCCGAG | |
| | GCCGAGGUGCAGAUCGACCGGCUGAUCACUGGCCGGCUG | |
| | CAGAGCCUGCAGACCUACGUGACCCAGCAGCUGAUCCGG | |
| | GCCGCCGAGAUUCGGGCCAGCGCCAACCUGGCCGCCACC | |
| | AAGAUGAGCGAGUGCGUGCUGGGCCAGAGCAAGCGGGUG | |
| | GACUUCUGCGGCAAGGGCUACCACCUGAUGAGCUUUCCC | |
| | CAGAGCGCACCCCACGGAGUGGUGUUCCUGCACGUGACC | |
| | UACGUGCCCGCCCAGGAGAAGAACUUCACCACCGCCCCA | |
| | GCCAUCUGCCACGACGGCAAGGCCCACUUUCCCCGGGAG | |
| | GGCGUGUUCGUGAGCAACGGCACCCACUGGUUCGUGACC | |
| | CAGCGGAACUUCUACGAGCCCCAGAUCAUCACCACCGAC | |
| | AACACCUUCGUGAGCGGCAACUGCGACGUGGUGAUCGGC | |
| | AUCGUGAACAACACCGUGUACGAUCCCCUGCAGCCCGAG | |
| | CUGGACAGCUUCAAGGAGGAGCUGGACAAGUACUUCAAG | |
| | AAUCACACCAGCCCCGACGUGGACCUGGGCGACAUCAGC | |
| | GGCAUCAACGCCAGCGUGGUGAACAUCCAGAAGGAGAUC | |
| | GAUCGGCUGAACGAGGUGGCCAAGAACCUGAACGAGAGC | |
| | CUGAUCGACCUGCAGGAGCUGGGCAAGUACGAGCAGUAC | |
| | AUCAAGUGGCCCUGGUACAUCUGGCUGGGCUUCAUCGCC | |
| | GGCCUGAUCGCCAUCGUGAUGGUGACCAUCAUGCUGUGC | |
| | UGCAUGACCAGCUGCUGCAGCUGCCUGAAGGGCUGUUGC | |
| | AGCUGCGGCAGCUGCUGCAAGUUCGACGAGGACGACAGC | |
| | GAGCCCGUGCUGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGGG | |
| | C | |
| Corresponding amino | MFVFLVLLPLVSSQCVNLTTRTQLPPAYTNSFTRGVYYPDKV | 5 |
| acid sequence | FRSSVLHSTQDLFLPFFSNVTWFHAIHVSGTNGTKRFDNPVLP | |
| | FNDGVYFASTEKSNIIRGWIFGTTLDSKTQSLLIVNNATNVVI | |
| | KVCEFQFCNDPFLGVYYHKNNKSWMESEFRVYSSANNCTFE | |
| | YVSQPFLMDLEGKQGNFKNLREFVFKNIDGYFKIYSKHTPINL | |
| | VRDLPQGFSALEPLVDLPIGINITRFQTLLALHRSYLTPGDSSS | |
| | GWTAGAAAYYVGYLQPRTFLLKYNENGTITDAVDCALDPLS | |
| | ETKCTLKSFTVEKGIYQTSNFRVQPTESIVRFPNITNLCPFGEV | |
| | FNATRFASVYAWNRKRISNCVADYSVLYNSASFSTFKCYGVS | |
| | PTKLNDLCFTNVYADSFVIRGDEVRQIAPGQTGKIADYNYKL | |
| | PDDFTGCVIAWNSNNLDSKVGGNYNYLYRLFRKSNLKPFER | |
| | DISTEIYQAGSTPCNGVEGFNCYFPLQSYGFQPTNGVGYQPYR | |
| | VVVLSFELLHAPATVCGPKKSTNLVKNKCVNFNFNGLTGTG | |
| | VLTESNKKFLPFQQFGRDIADTTDAVRDPQTLEILDITPCSFGG | |
| | VSVITPGTNTSNQVAVLYQDVNCTEVPVAIHADQLTPTWRV | |
| | YSTGSNVFQTRAGCLIGAEHVNNSYECDIPIGAGICASYQTQT | |
| | NSPRRARSVASQSIIAYTMSLGAENSVAYSNNSIAIPTNFTISV | |
| | TTEILPVSMTKTSVDCTMYICGDSTECSNLLLQYGSFCTQLNR | |
| | ALTGIAVEQDKNTQEVFAQVKQIYKTPPIKDFGGFNFSQILPD | |
| | PSKPSKRSFIEDLLFNKVTLADAGFIKQYGDCLGDIAARDLIC | |
| | AQKFNGLTVLPPLLTDEMIAQYTSALLAGTITSGWTFGAGAA | |
| | LQIPFAMQMAYRFNGIGVTQNVLYENQKLIANQFNSAIGKIQ | |
| | DSLSSTASALGKLQDVVNQNAQALNTLVKQLSSNFGAISSVL | |
| | NDILSRLDPPEAEVQIDRLITGRLQSLQTYVTQQLIRAAEIRAS | |
| | ANLAATKMSECVLGQSKRVDFCGKGYHLMSFPQSAPHGVVF | |
| | LHVTYVPAQEKNFTTAPAICHDGKAHFPREGVFVSNGTHWF | |
| | VTQRNFYEPQIITTDNTFVSGNCDVVIGIVNNTVYDPLQPELD | |
| | SFKEELDKYFKNHTSPDVDLGDISGINASVVNIQKEIDRLNEV | |
| | AKNLNESLIDLQELGKYEQYIKWPWYIWLGFIAGLIAIVMVTI | |
| | MLCCMTSCCSCLKGCCSCGSCCKFDEDDSEPVLKGVKLHYT | |
| Poly A tail | 100 nt | |

B.1.1.529-1273_m_mu

| SEQ ID NO: 6 consists of from 5′ end to 3′ end: 5′ UTR SEQ ID NO: 2, | | 6 |
|---|---|---|
| mRNA ORF SEQ ID NO: 7, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 7 |
| Construct | CAGUGCGUGAACCUGACCACCCGGACCCAGCUGCCACCA | |
| (excluding the stop | GCCUACACCAACAGCUUCACCCGGGGCGUGUACUACCCC | |
| codon) | GACAAGGUGUUCCGGAGCAGCGUGCUGCACAGCACCCAG | |
| | GACCUGUUCCUGCCCUUCUUCAGCAACGUGACCUGGUUC | |
| | CACGUGAUCAGCGGCACCAACGGCACCAAGCGGUUCGAC | |
| | AACCCCGUGCUGCCCUUCAACGACGGCGUGUACUUCGCC | |
| | AGCAUCGAGAAGAGCAACAUCAUCCGGGGCUGGAUCUUC | |
| | GGCACCACCCUGGACAGCAAGACCCAGAGCCUGCUGAUC | |
| | GUGAACAACGCCACCAACGUGGUGAUCAAGGUGUGCGAG | |
| | UUCCAGUUCUGCAACGACCCCUUCCUGGACCACAAGAAC | |
| | AACAAGAGCUGGAUGGAGAGCGAGUUCCGGGUGUACAGC | |
| | AGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAUCGUGCGG | |
| | GAGCCCGAGGACCUGCCCCAGGGCUUCAGCGCCCUGGAG | |
| | CCCCUGGUGGACCUGCCCAUCGGCAUCAACAUCACCCGG | |
| | UUCCAGACCCUGCUGGCCCUGCACCGGAGCUACCUGACC | |
| | CCAGGCGACAGCAGCAGCGGCUGGACCGCCGGCGCCGCC | |
| | GCCUACUACGUGGGCUACCUGCAGCCCCGGACCUUCCUG | |
| | CUGAAGUACAACGAGAACGGCACCAUCACCGACGCCGUG | |
| | GACUGCGCCCUGGACCCUCUGAGCGAGACCAAGUGCACC | |
| | CUGAAGAGCUUCACCGUGGAGAAGGGCAUCUACCAGACC | |
| | AGCAACUUCCGGGUGCAGCCCACCGAGAGCAUCGUGCGG | |
| | UUCCCCAACAUCACCAACCUGUGCCCCUUCGACGAGGUG | |
| | UUCAACGCCACCCGGUUCGCCAGCGUGUACGCCUGGAAC | |
| | CGGAAGCGGAUCAGCAACUGCGUGGCCGACUACAGCGUG | |
| | CUGUACAACCUGGCCCCUUUCUUCACCUUCAAGUGCUAC | |
| | GGCGUGAGCCCCACCAAGCUGAACGACCUGUGCUUCACC | |
| | AACGUGUACGCCGACAGCUUCGUGAUCCGGGGCGACGAG | |
| | GUGCGGCAGAUCGCCCCAGGCCAGACCGGCAACAUCGCC | |
| | GACUACAACUACAAGCUGCCCGACGACUUCACCGGCUGC | |
| | GUGAUCGCCUGGAACAGCAACAAGCUGGACAGCAAGGUG | |
| | AGCGGCAACUACAACUACCUGUACCGGCUGUUCCGGAAG | |
| | AGCAACCUGAAGCCCUUCGAGCGGGACAUCAGCACCGAG | |
| | AUCUACCAGGCCGGCAACAAGCCCUGCAACGGCGUGGCC | |
| | GGCUUCAACUGCUACUUCCCUCUGCGGAGCUACAGCUUC | |
| | CGGCCCACCUACGGCGUGGGCCACCAGCCCUACCGGGUG | |
| | GUGGUGCUGAGCUUCGAGCUGCUGCACGCCCCAGCCACC | |
| | GUGUGCGGCCCCAAGAAGAGCACCAACCUGGUGAAGAAC | |
| | AAGUGCGUGAACUUCAACUUCAACGGCCUGAAGGGCACC | |
| | GGCGUGCUGACCGAGAGCAACAAGAAGUUCCUGCCCUUC | |
| | CAGCAGUUCGGCCGGGACAUCGCCGACACCACCGACGCC | |
| | GUGCGGGAUCCCCAGACCCUGGAGAUCCUGGACAUCACC | |
| | CCUUGCAGCUUCGGCGGCGUGAGCGUGAUCACCCCAGGC | |
| | ACCAACACCAGCAACCAGGUGGCCGUGCUGUACCAGGGC | |
| | GUGAACUGCACCGAGGUGCCCGUGGCCAUCCACGCCGAC | |
| | CAGCUGACCCCAACCUGGCGGGUGUACAGCACCGGCAGC | |
| | AACGUGUUCCAGACCCGGGCCGGCUGCCUGAUCGGCGCC | |
| | GAGUACGUGAACAACAGCUACGAGUGCGACAUCCCCAUC | |
| | GGCGCCGGCAUCUGCGCCAGCUACCAGACCCAGACCAAG | |
| | AGCCACCGGCGGGCCCGGAGCGUGGCCAGCCAGAGCAUC | |
| | AUCGCCUACACCAUGAGCCUGGGCGCCGAGAACAGCGUG | |
| | GCCUACAGCAACAACAGCAUCGCCAUCCCCACCAACUUC | |
| | ACCAUCAGCGUGACCACCGAGAUCCUGCCCGUGAGCAUG | |
| | ACCAAGACCAGCGUGGACUGCACCAUGUACAUCUGCGGC | |
| | GACAGCACCGAGUGCAGCAACCUGCUGCUGCAGUACGGC | |
| | AGCUUCUGCACCCAGCUGAAGCGGGCCCUGACCGGCAUC | |
| | GCCGUGGAGCAGGACAAGAACACCCAGGAGGUGUUCGCC | |
| | CAGGUGAAGCAGAUCUACAAGACCCCUCCCAUCAAGUAC | |
| | UUCGGCGGCUUCAACUUCAGCCAGAUCCUGCCCGACCCC | |
| | AGCAAGCCCAGCAAGCGGAGCUUCAUCGAGGACCUGCUG | |
| | UUCAACAAGGUGACCCUGGCCGACGCCGGCUUCAUCAAG | |
| | CAGUACGGCGACUGCCUGGGCGACAUCGCCGCCCGGGAC | |
| | CUGAUCUGCGCCCAGAAGUUCAAGGGCCUGACCGUGCUG | |
| | CCUCCUCUGCUGACCGACGAGAUGAUCGCCCAGUACACC | |
| | AGCGCCCUGCUGGCCGGCACCAUCACCAGCGGCUGGACC | |
| | UUCGGCGCCGGCGCCGCCCUGCAGAUCCCCUUCGCCAUGC | |
| | AGAUGGCCUACCGGUUCAACGGCAUCGGCGUGACCCAGA | |
| | ACGUGCUGUACGAGAACCAGAAGCUGAUCGCCAACCAGU | |
| | UCAACAGCGCCAUCGGCAAGAUCCAGGACAGCCUGAGCA | |
| | GCACCGCCAGCGCCCUGGGCAAGCUGCAGGACGUGGUGA | |
| | ACCACAACGCCCAGGCCCUGAACACCCUGGUGAAGCAGC | |
| | UGAGCAGCAAGUUCGGCGCCAUCAGCAGCGUGCUGAACG | |
| | ACAUCUUCAGCCGGCUGGACCCUCCCGAGGCCGAGGUGC | |
| | AGAUCGACCGGCUGAUCACCGGCCGGCUGCAGAGCCUGC | |
| | AGACCUACGUGACCCAGCAGCUGAUCCGGGCCGCCGAGA | |
| | UCCGGGCCAGCGCCAACCUGGCCGCCACCAAGAUGAGCG | |
| | AGUGCGUGCUGGGCCAGAGCAAGCGGGUGGACUUCUGCG | |
| | GCAAGGGCUACCACCUGAUGAGCUUUCCCCAGAGCGCAC | |
| | CCCACGGCGUGGUGUUCCUGCACGUGACCUACGUGCCCG | |
| | CCCAGGAGAAGAACUUCACCACCGCCCCAGCCAUCUGCC | |
| | ACGACGGCAAGGCCCACUUUCCCCGGGAGGGCGUGUUCG | |
| | UGAGCAACGGCACCCACUGGUUCGUGACCCAGCGGAACU | |
| | UCUACGAGCCCCAGAUCAUCACCACCGACAACACCUUCG | |
| | UGAGCGGCAACUGCGACGUGGUGAUCGGCAUCGUGAACA | |
| | ACACCGUGUACGAUCCCCUGCAGCCCGAGCUGGACAGCU | |
| | UCAAGGAGGAGCUGGACAAGUACUUCAAGAACCACACCA | |
| | GCCCCGACGUGGACCUGGGCGACAUCAGCGGCAUCAACG | |
| | CCAGCGUGGUGAACAUCCAGAAGGAGAUCGACAGACUGA | |
| | ACGAGGUGGCCAAGAACCUGAACGAGAGCCUGAUCGACC | |
| | UGCAGGAGCUGGGCAAGUACGAGCAGUACAUCAAGUGGC | |
| | CCUGGUACAUCUGGCUGGGCUUCAUCGCCGGCCUGAUCG | |
| | CCAUCGUGAUGGUGACCAUCAUGCUGUGCUGCAUGACCA | |
| | GCUGCUGCAGCUGCCUGAAGGGCUGCUGCAGCUGCGGCA | |
| | GCUGCUGCAAGUUCGACGAGGACGACAGCGAGCCCGUGC | |
| | UGAAGGGCGUGAAGCUGCACUACACC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNLTTRTQLPPAYTNSFTRGVYYPDKV | 8 |
| acid sequence | FRSSVLHSTQDLFLPFFSNVTWFHVISGTNGTKRFDNPVLPFN | |
| | DGVYFASIEKSNIIRGWIFGTTLDSKTQSLLIVNNATNVVIKVC | |
| | EFQFCNDPFLDHKNNKSWMESEFRVYSSANNCTFEYVSQPFL | |
| | MDLEGKQGNFKNLREFVFKNIDGYFKIYSKHTPIIVREPEDLP | |
| | QGFSALEPLVDLPIGINITRFQTLLALHRSYLTPGDSSSGWTAG | |
| | AAAYYVGYLQPRTFLLKYNENGTITDAVDCALDPLSETKCTL | |
| | KSFTVEKGIYQTSNFRVQPTESIVRFPNITNLCPFDEVFNATRF | |
| | ASVYAWNRKRISNCVADYSVLYNLAPFFTFKCYGVSPTKLN | |
| | DLCFTNVYADSFVIRGDEVRQIAPGQTGNIADYNYKLPDDFT | |
| | GCVIAWNSNKLDSKVSGNYNYLYRLFRKSNLKPFERDISTEIY | |
| | QAGNKPCNGVAGFNCYFPLRSYSFRPTYGVGHQPYRVVVLS | |
| | FELLHAPATVCGPKKSTNLVKNKCVNFNFNGLKGTGVLTESN | |
| | KKFLPFQQFGRDIADTTDAVRDPQTLEILDITPCSFGGVSVITP | |
| | GTNTSNQVAVLYQGVNCTEVPVAIHADQLTPTWRVYSTGSN | |
| | VFQTRAGCLIGAEYVNNSYECDIPIGAGICASYQTQTKSHRRA | |
| | RSVASQSIIAYTMSLGAENSVAYSNNSIAIPTNFTISVTTEILPV | |
| | SMTKTSVDCTMYICGDSTECSNLLLQYGSFCTQLKRALTGIA | |
| | VEQDKNTQEVFAQVKQIYKTPPIKYFGGFNFSQILPDPSKPSK | |
| | RSFIEDLLFNKVTLADAGFIKQYGDCLGDIAARDLICAQKFKG | |
| | LTVLPPLLTDEMIAQYTSALLAGTITSGWTFGAGAALQIPFAM | |
| | QMAYRFNGIGVTQNVLYENQKLIANQFNSAIGKIQDSLSSTAS | |
| | ALGKLQDVVNHNAQALNTLVKQLSSKFGAISSVLNDIFSRLD | |
| | PPEAEVQIDRLITGRLQSLQTYVTQQLIRAAEIRASANLAATK | |
| | MSECVLGQSKRVDFCGKGYHLMSFPQSAPHGVVFLHVTYVP | |
| | AQEKNFTTAPAICHDGKAHFPREGVFVSNGTHWFVTQRNFY | |
| | EPQIITTDNTFVSGNCDVVIGIVNNTVYDPLQPELDSFKEELDK | |
| | YFKNHTSPDVDLGDISGINASVVNIQKEIDRLNEVAKNLNESL | |
| | IDLQELGKYEQYIKWPWYIWLGFIAGLIAIVMVTIMLCCMTSC | |
| | CSCLKGCCSCGSCCKFDEDDSEPVLKGVKLHYT | |
| PolyA tail | 100 nt | |

BA.4/5

| SEQ ID NO: 6 consists of from 5′ end to 3′ end: 5′ UTR SEQ ID NO: 2, | | 9 |
|---|---|---|
| mRNA ORF SEQ ID NO: 10, and 3′ UTR SEQ ID NO: 11. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 10 |
| Construct | CAGUGCGUGAACCUGAUCACCCGGACCCAGAGCUACACCA | |
| (excluding the stop | ACAGCUUCACCCGGGGCGUCUACUACCCCGACAAGGUGU | |
| codon) | UCCGGAGCAGCGUCCUGCACAGCACCCAGGACCUGUUCCU | |
| | GCCCUUCUUCAGCAACGUGACCUGGUUCCACGCCAUCAGC | |
| | GGCACCAACGGCACCAAGCGGUUCGACAACCCCGUGCUGC | |
| | CCUUCAACGACGGCGUGUACUUCGCCAGCACCGAGAAGA | |
| | GCAACAUCAUCCGGGGCUGGAUCUUCGGCACCACCCUGG | |
| | ACAGCAAGACCCAGAGCCUGCUGAUCGUGAAUAACGCCA | |
| | CCAACGUGGUGAUCAAGGUGUGCGAGUUCCAGUUCUGCA | |
| | ACGACCCCUUCCUGGACGUGUACUACCACAAGAACAACA | |
| | AGAGCUGGAUGGAGAGCGAGUUCCGGGUGUACAGCAGCG | |
| | CCAACAACUGCACCUUCGAGUACGUGAGCCAGCCCUUCCU | |
| | GAUGGACCUGGAGGGCAAGCAGGGCAACUUCAAGAACCU | |
| | GCGGGAGUUCGUGUUCAAGAACAUCGACGGCUACUUCAA | |
| | GAUCUACAGCAAGCACACCCCAAUCAACCUGGGCCGGGA | |
| | UCUGCCCCAGGGCUUCUCAGCCCUGGAGCCCCUGGUGGAC | |
| | CUGCCCAUCGGCAUCAACAUCACCCGGUUCCAGACCCUGC | |
| | UGGCCCUGCACCGGAGCUACCUGACCCCAGGCGACAGCAG | |
| | CAGCGGGUGGACAGCAGGCGCGGCUGCUUACUACGUGGG | |
| | CUACCUGCAGCCCCGGACCUUCCUGCUGAAGUACAACGAG | |
| | AACGGCACCAUCACCGACGCCGUGGACUGCGCCCUGGACC | |
| | CUCUGAGCGAGACCAAGUGCACCCUGAAGAGCUUCACCG | |
| | UGGAGAAGGGCAUCUACCAGACCAGCAACUUCCGGGUGC | |
| | AGCCCACCGAGAGCAUCGUGCGGUUCCCCAACAUCACCAA | |
| | CCUGUGCCCCUUCGACGAGGUGUUCAACGCCACCCGGUUC | |
| | GCCAGCGUGUACGCCUGGAACCGGAAGCGGAUCAGCAAC | |
| | UGCGUGGCCGACUACAGCGUGCUGUACAACUUCGCCCCA | |
| | UUCUUCGCCUUCAAGUGCUACGGCGUGAGCCCCACCAAGC | |
| | UGAACGACCUGUGCUUCACCAACGUGUACGCCGACAGCU | |
| | UCGUGAUCCGUGGCAACGAGGUGAGCCAGAUCGCACCCG | |
| | GCCAGACAGGCAACAUCGCCGACUACAACUACAAGCUGCC | |
| | CGACGACUUCACCGGCUGCGUGAUCGCCUGGAACAGCAA | |
| | CAAGCUCGACAGCAAGGUGGGCGGCAACUACAACUACCG | |
| | GUACCGGCUGUUCCGGAAGAGCAACCUGAAGCCCUUCGA | |
| | GCGGGACAUCAGCACCGAGAUCUACCAAGCCGGCAACAA | |
| | GCCUUGCAACGGCGUGGCCGGCGUGAACUGCUACUUCCC | |
| | UCUGCAGAGCUACGGCUUCCGGCCCACCUACGGCGUGGGC | |
| | CACCAGCCCUACCGGGUGGUGGUGCUGAGCUUCGAGCUG | |
| | CUGCACGCCCCAGCCACCGUGUGUGGCCCCAAGAAGAGCA | |
| | CCAACCUGGUGAAGAACAAGUGCGUGAACUUCAACUUCA | |
| | ACGGCCUUACCGGCACCGGCGUGCUGACCGAGAGCAACA | |
| | AGAAAUUCCUGCCCUUUCAGCAGUUCGGCCGGGACAUCG | |
| | CCGACACCACCGACGCUGUGCGGGAUCCCCAGACCCUGGA | |
| | GAUCCUGGACAUCACCCCUUGCAGCUUCGGCGGCGUGAG | |
| | CGUGAUCACCCCAGGCACCAACACCAGCAACCAGGUGGCC | |
| | GUGCUGUACCAGGGUGUGAACUGCACCGAGGUGCCCGUG | |
| | GCCAUCCACGCCGACCAGCUGACACCCACCUGGCGGGUCU | |
| | ACAGCACCGGCAGCAACGUGUUCCAGACCCGGGCCGGUU | |
| | GCCUGAUCGGCGCCGAGUACGUGAACAACAGCUACGAGU | |
| | GCGACAUCCCCAUCGGCGCCGGCAUCUGUGCCAGCUACCA | |
| | GACCCAGACCAAGUCACACCGGAGGGCAAGGAGCGUGGC | |
| | CAGCCAGAGCAUCAUCGCCUACACCAUGAGCCUGGGCGCC | |
| | GAGAACAGCGUGGCCUACAGCAACAACAGCAUCGCCAUC | |
| | CCCACCAACUUCACCAUCAGCGUGACCACCGAGAUUCUGC | |
| | CCGUGAGCAUGACCAAGACCAGCGUGGACUGCACCAUGU | |
| | ACAUCUGCGGCGACAGCACCGAGUGCAGCAACCUGCUGC | |
| | UGCAGUACGGCAGCUUCUGCACCCAGCUGAAGCGGGCCC | |
| | UGACCGGCAUCGCCGUGGAGCAGGACAAGAACACCCAGG | |
| | AGGUGUUCGCCCAGGUGAAGCAGAUCUACAAGACCCCUC | |
| | CCAUCAAGUACUUCGGCGGCUUCAACUUCAGCCAGAUCC | |
| | UGCCCGACCCCAGCAAGCCCAGCAAGCGGAGCUUCAUCGA | |
| | GGACCUGCUGUUCAACAAGGUGACCCUAGCCGACGCCGG | |
| | CUUCAUCAAGCAGUACGGCGACUGCCUCGGCGACAUAGC | |
| | CGCCCGGGACCUGAUCUGCGCCCAGAAGUUCAACGGCCUG | |
| | ACCGUGCUGCCUCCCCUGCUGACCGACGAGAUGAUCGCCC | |
| | AGUACACCAGCGCCCUGUUAGCCGGAACCAUCACCAGCGG | |
| | CUGGACUUUCGGCGCUGGAGCCGCUCUGCAGAUCCCCUUC | |
| | GCCAUGCAGAUGGCCUACCGGUUCAACGGCAUCGGCGUG | |
| | ACCCAGAACGUGCUGUACGAGAACCAGAAGCUGAUCGCC | |
| | AACCAGUUCAACAGCGCCAUCGGCAAGAUCCAGGACAGC | |
| | CUGAGCAGCACCGCUAGCGCCCUGGGCAAGCUGCAGGAC | |
| | GUGGUGAACCACAACGCCCAGGCCCUGAACACCCUGGUG | |
| | AAGCAGCUGAGCAGCAAGUUCGGCGCCAUCAGCAGCGUG | |
| | CUGAACGACAUCCUGAGCCGGCUGGACCCUCCCGAGGCCG | |
| | AGGUGCAGAUCGACCGGCUGAUCACUGGCCGGCUGCAGA | |
| | GCCUGCAGACCUACGUGACCCAGCAGCUGAUCCGGGCCGC | |
| | CGAGAUUCGGGCCAGCGCCAACCUGGCCGCCACCAAGAUG | |
| | AGCGAGUGCGUGCUGGGCCAGAGCAAGCGGGUGGACUUC | |
| | UGCGGCAAGGGCUACCACCUGAUGAGCUUUCCCCAGAGC | |
| | GCACCCCACGGAGUGGUGUUCCUGCACGUGACCUACGUG | |
| | CCCGCCCAGGAGAAGAACUUCACCACCGCCCCAGCCAUCU | |
| | GCCACGACGGCAAGGCCCACUUUCCCCGGGAGGGCGUGU | |
| | UCGUGAGCAACGGCACCCACUGGUUCGUGACCCAGCGGA | |
| | ACUUCUACGAGCCCCAGAUCAUCACCACCGACAACACCUU | |
| | CGUGAGCGGCAACUGCGACGUGGUGAUCGGCAUCGUGAA | |
| | CAACACCGUGUACGAUCCCCUGCAGCCCGAGCUGGACAGC | |
| | UUCAAGGAGGAGCUGGACAAGUACUUCAAGAAUCACACC | |
| | AGCCCCGACGUGGACCUGGGCGACAUCAGCGGCAUCAAC | |
| | GCCAGCGUGGUGAACAUCCAGAAGGAGAUCGAUCGGCUG | |
| | AACGAGGUGGCCAAGAACCUGAACGAGAGCCUGAUCGAC | |
| | CUGCAGGAGCUGGGCAAGUACGAGCAGUACAUCAAGUGG | |
| | CCCUGGUACAUCUGGCUGGGCUUCAUCGCCGGCCUGAUC | |
| | GCCAUCGUGAUGGUGACCAUCAUGCUGUGCUGCAUGACC | |
| | AGCUGCUGCAGCUGCCUGAAGGGCUGUUGCAGCUGCGGC | |
| | AGCUGCUGCAAGUUCGACGAGGACGACAGCGAGCCCGUG | |
| | CUGAAGGGCGUGAAGCUGCACUACACC | |
| 3′ UTR | UGAUAAUAGGCUGGAGCCUCGGUGGCCUAGCUUCUUGCC | 11 |
| | CCUUGGGCCUCCCCCCAGCCCCUCCUCCCCUUCCUGCAGG | |
| | AGAGAUAGUGGAGUGGUCUUUGAAUAAAGUCUGAGUGGG | |
| | CGGC | |
| Corresponding amino | MFVFLVLLPLVSSQCVNLITRTQSYTNSFTRGVYYPDKVFRSS | 12 |
| acid sequence | VLHSTQDLFLPFFSNVTWFHAISGTNGTKRFDNPVLPFNDGVY | |
| | FASTEKSNIIRGWIFGTTLDSKTQSLLIVNNATNVVIKVCEFQF | |
| | CNDPFLDVYYHKNNKSWMESEFRVYSSANNCTFEYVSQPFL | |
| | MDLEGKQGNFKNLREFVFKNIDGYFKIYSKHTPINLGRDLPQG | |
| | FSALEPLVDLPIGINITRFQTLLALHRSYLTPGDSSSGWTAGAA | |
| | AYYVGYLQPRTFLLKYNENGTITDAVDCALDPLSETKCTLKSF | |
| | TVEKGIYQTSNFRVQPTESIVRFPNITNLCPFDEVFNATRFASV | |
| | YAWNRKRISNCVADYSVLYNFAPFFAFKCYGVSPTKLNDLCF | |
| | TNVYADSFVIRGNEVSQIAPGQTGNIADYNYKLPDDFTGCVIA | |
| | WNSNKLDSKVGGNYNYRYRLFRKSNLKPFERDISTEIYQAGN | |
| | KPCNGVAGVNCYFPLQSYGFRPTYGVGHQPYRVVVLSFELLH | |
| | APATVCGPKKSTNLVKNKCVNFNFNGLTGTGVLTESNKKFLP | |
| | FQQFGRDIADTTDAVRDPQTLEILDITPCSFGGVSVITPGTNTS | |
| | NQVAVLYQGVNCTEVPVAIHADQLTPTWRVYSTGSNVFQTR | |
| | AGCLIGAEYVNNSYECDIPIGAGICASYQTQTKSHRRARSVAS | |
| | QSIIAYTMSLGAENSVAYSNNSIAIPTNFTISVTTEILPVSMTKT | |
| | SVDCTMYICGDSTECSNLLLQYGSFCTQLKRALTGIAVEQDK | |
| | NTQEVFAQVKQIYKTPPIKYFGGFNFSQILPDPSKPSKRSFIEDL | |
| | LFNKVTLADAGFIKQYGDCLGDIAARDLICAQKFNGLTVLPPL | |
| | LTDEMIAQYTSALLAGTITSGWTFGAGAALQIPFAMQMAYRF | |
| | NGIGVTQNVLYENQKLIANQFNSAIGKIQDSLSSTASALGKLQ | |
| | DVVNHNAQALNTLVKQLSSKFGAISSVLNDILSRLDPPEAEVQ | |
| | IDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNT | |
| | FVSGNCDVVIGIVNNTVYDPLQPELDSFKEELDKYFKNHTSPD | |
| | VDLGDISGINASVVNIQKEIDRLNEVAKNLNESLIDLQELGKYE | |
| | QYIKWPWYIWLGFIAGLIAIVMVTIMLCCMTSCCSCLKGCCSC | |
| | GSCCKFDEDDSEPVLKGVKLHYT | |
| Poly A tail | 100 nt | |

TABLE 7
5′ UTR sequences

| SEQ ID | |
|---|---|
| NO: | Sequence |
| 18 | GGAAAUCGCAAAAUUUGCUCUUCGCGUUAGAUUUCUUUUAGUUUUCUCGCAACUAGCAAGCUU |
| | UUUGUUCUCGCC |
| 19 | GGAAAUCCCCACAACCGCCUCAUAUCCAGGCUCAAGAAUAGAGCUCAGUGUUUUGUUGUUUAA |
| | UCAUUCCGACGUGUUUUGCGAUAUUCGCGCAAAGCAGCCAGUCGCGCGCUUGCUUUUAAGUAG |
| | AGUUGUUUUUCCACCCGUUUGCCAGGCAUCUUUAAUUUAACAUAUUUUUAUUUUUCAGGCUAA |
| | CCUACGCCGCCACC |
| 20 | GGAAAUAAGAGAGAAAAGAAGAGUAAGAAGAAAUAUAAGAUCUCCCUGAGCUUCAGGGAGCCC |
| | CGGCGCCGCCACC |
| 21 | GGAAACCCCCCACCCCCGUAAGAGAGAAAAGAAGAGUAAGAAGAAAUAUAAGAUCUCCCUGAG |
| | CUUCAGGGAGCCCCGGCGCCGCCACC |
| 22 | GGAGAACUUCCGCUUCCGUUGGCGCAAGCGCUUUCAUUUUUUCUGCUACCGUGACUAAG |
| 23 | GGAAAUAAGAGAGAAAAGAAGAGUAAGAAGAAAUAUAAGAGCCACC |
| 24 | GGAAAUAAGAGAGAAAAGAAGAGUAAGAAGAAAUAUAAGACCCCGGCGCCGCCACC |
| 25 | GGAAAUCGCAAAAUUUGCUCUUCGCGUUAGAUUUCUUUUAGUUUUCUCGCAACUAGCAAGCUU |
| | UUUGUUCUCGCCGCCGCC |
| 26 | GGAAAUCGCAAAAUUUUCUUUUCGCGUUAGAUUUCUUUUAGUUUUCUUUCAACUAGCAAGCUU |
| | UUUGUUCUCGCCGCCGCC |
| 27 | G G A A A U C G C A A A A (N₂)ₓ (N₃)ₓ C U (N₄)ₓ (N₅)ₓ C G C G U U A |
| | G A U U U C U U U U A G U U U U C U N₆ N₇ C A A C U A G C A A G C |
| | U U U U U G U U C U C G C C (N₈ C C)x |
| | (N₂)ₓ is a uracil and x is an integer from 0 to 5, e.g., wherein x = 3 or 4; |
| | (N₃)ₓ is a guanine and x is an integer from 0 to 1; |
| | (N₄)ₓ is a cytosine and x is an integer from 0 to 1; |
| | (N₅)ₓ is a uracil and x is an integer from 0 to 5, e.g., wherein x = 2 or 3; |
| | N₆ is a uracil or cytosine; |
| | N₇ is a uracil or guanine; |
| | N₈ is adenine or guanine and x is an integer from 0 to 1. |
| 28 | GGAAAAUUUUAGCCUGGAACGUUAGAUAACUGUCCUGUUGUCUUUAUAUACUUGGUCCCCAAG |
| | UAGUUUGUCUUCCAAA |
| 29 | GGAAACUUUAUUUAGUGUUACUUUAUUUUCUGUUUAUUUGUGUUUCUUCAGUGGGUUUGUUCU |
| | AAUUUCCUUGGCCGCC |
| 30 | GGAAAAUCUGUAUUAGGUUGGCGUGUUCUUUGGUCGGUUGUUAGUAUUGUUGUUGAUUCGUUU |
| | GUGGUCGGUUGCCGCC |
| 31 | GGAAAAUUAUUAACAUCUUGGUAUUCUCGAUAACCAUUCGUUGGAUUUUAUUGUAUUCGUAGU |
| | UUGGGUUCCUGCCGCC |
| 32 | GGAAAUUAUUAUUAUUUCUAGCUACAAUUUAUCAUUGUAUUAUUUUAGCUAUUCAUCAUUAUU |
| | UACUUGGUGAUCAACA |
| 33 | GGAAAUAGGUUGUUAACCAAGUUCAAGCCUAAUAAGCUUGGAUUCUGGUGACUUGCUUCACCG |
| | UUGGCGGGCACCGAUC |
| 34 | GGAAAUCGUAGAGAGUCGUACUUAGUACAUAUCGACUAUCGGUGGACACCAUCAAGAUUAUAA |
| | ACCAGGCCAGA |
| 35 | GGAAACCCGCCCAAGCGACCCCAACAUAUCAGCAGUUGCCCAAUCCCAACUCCCAACACAAUC |
| | CCCAAGCAACGCCGCC |
| 36 | GGAAAGCGAUUGAAGGCGUCUUUUCAACUACUCGAUUAAGGUUGGGUAUCGUCGUGGGACUUG |
| | GAAAUUUGUUGUUUCC |
| 37 | GGAAACUAAUCGAAAUAAAAGAGCCCCGUACUCUUUUAUUUCUAUUAGGUUAGGAGCCUUAGC |
| | AUUUGUAUCUUAGGUA |
| 38 | GGAAAUGUGAUUUCCAGCAACUUCUUUUGAAUAUAUUGAAUUCCUAAUUCAAAGCGAACAAAU |
| | CUACAAGCCAUAUACC |
| 39 | GGAAAUCGUAGAGAGUCGUACUUACGUGGUCGCCAUUGCAUAGCGCGCGAAAGCAACAGGAAC |
| | AAGAACGCGCC |
| 40 | GGAAAUCGUAGAGAGUCGUACUUAGAAUAAACAGAGUCGGGUCGACUUGUCUCUGAUACUACG |
| | ACGUCACAAUC |
| 41 | GGAAAAUUUGCCUUCGGAGUUGCGUAUCCUGAACUGCCCAGCCUCCUGAUAUACAACUGUUCC |
| | GCUUAUUCGGGCCGCC |
| 42 | GGAAAUCUGAGCAGGAAUCCUUUGUGCAUUGAAGACUUUAGAUUCCUCUCUGCGGUAGACGUG |
| | CACUUAUAAGUAUUUG |
| 43 | GGAAAGCGAUUGAAGGCGUCUUUUCAACUACUCGAUUAAGGUUGGGUAUCGUCGUGGGACUUG |
| | GAAAUUUGUUGCCACC |
| 44 | GGAAAUUUUUUUUUGAUAUUAUAAGAGUUUUUUUUUGAUAUUAAGAAAAUUUUUUUUUGAUAU |
| | UAGAAGAGUAAGAAGAAAUAUAAGACCCCGGCGCCGCCACC |
| 45 | GGAAAUAAGAGAGAAAAGAAGAGUAAGAAGAAAUAUAAGAGCCAAAAAAAAAAAACC |
| 46 | GGAAAUCUCCCUGAGCUUCAGGGAGUAAGAGAGAAAAGAAGAGUAAGAAGAAAUAUAAGACCC |
| | CGGCGCCGCCACC |
| | GCCRCC, wherein R = A or G |
| 47 | GGACUCACUAUUUGUUUUCGCGCCCAGUUGCAAAAA |

TABLE 8
3′ UTR sequences

| SEQ ID | |
|---|---|
| NO: | Sequence |
| 48 | UAAAGCUCCCCGGGGGCCUCGGUGGCCUAGCUUCUUGCCCCUUGGGCCUCCCCCCAGCCCCUCC |
| | UCCCCUUCCUGCACCCGUACCCCCGUGGUCUUUGAAUAAAGUCUGAGUGGGCGGC |
| 49 | UAAGUCUAAGCUGGAGCCUCCUGAGAGACCUGUGUGAACUAUUGAGAAGAUCGGAACAGCUCCU |
| | UACUCUGAGGAAGUUGGUACCCCCGUGGUCUUUGAAUAAAGUCUGAGUGGGCGGC |
| 50 | UAAAGCUCCCCGGGGCAAACACCAUUGUCACACUCCAGCCUCGGUGGCCUAGCUUCUUGCCCCU |
| | UGGGCCCAAACACCAUUGUCACACUCCAUCCCCCCAGCCCCUCCUCCCCUUCCUGCACCCGUAC |
| | CCCCCAAACACCAUUGUCACACUCCAGUGGUCUUUGAAUAAAGUCUGAGUGGGCGGC |
| | (miR122 binding sites boldened) |
| 51 | UAAAGCUCCCCGGGGUCCAUAAAGUAGGAAACACUACAGCUGGAGCCUCGGUGGCCUAGCUUCU |
| | UGCCCCUUGGGCCCAAACACCAUUGUCACACUCCAUCCCCCCAGCCCCUCCUCCCCUUCCUGCA |
| | CCCGUACCCCCGUGGUCUUUGAAUAAAGUCUGAGUGGGCGGC |
| | (miR-142-3p and miR122 binding sites boldened) |
| 52 | UAAAGCUCCCCGGGGGCCUCGGUGGCCUAGCUUCUUGCCCCUUGGGCCUCCCCCCAGCCCCUCC |
| | UCCCCUUCCUGCACCCGUACCCCCCAAACACCAUUGUCACACUCCAGUGGUCUUUGAAUAAAGU |
| | CUGAGUGGGCGGC |
| | (miR122 binding site boldened) |
| 53 | UAAGCCCCUCCGGGGCAAACACCAUUGUCACACUCCAGCCUCGGUGGCCUAGCUUCUUGCCCCU |
| | UGGGCCCAAACACCAUUGUCACACUCCAUCCCCCCAGCCCCUCCUCCCCUUCCUGCACCCGUAC |
| | CCCCCAAACACCAUUGUCACACUCCAGUGGUCUUUGAAUAAAGUCUGAGUGGGCGGC |
| | (miR122 binding sites boldened) |
| 54 | UAAGCCCCUCCGGGGUCCAUAAAGUAGGAAACACUACAGCCUCGGUGGCCUAGCUUCUUGCCCC |
| | UUGGGCCUCCAUAAAGUAGGAAACACUACAUCCCCCCAGCCCCUCCUCCCCUUCCUGCACCCGU |
| | ACCCCCCGCAUUAUUACUCACGGUACGAGUGGUCUUUGAAUAAAGUCUGAGUGGGCGGC |
| | (miR-142-3p and miR-126-3p binding sites boldened) |
| 55 | UAAGCCCCUCCGGGGUCCAUAAAGUAGGAAACACUACAGCUGGAGCCUCGGUGGCCUAGCUUCU |
| | UGCCCCUUGGGCCCAAACACCAUUGUCACACUCCAUCCCCCCAGCCCCUCCUCCCCUUCCUGCA |
| | CCCGUACCCCCGUGGUCUUUGAAUAAAGUCUGAGUGGGCGGC |
| | (miR-142-3p and miR122 binding sites boldened) |
| 56 | UAAGCCCCUCCGGGGGCCUCGGUGGCCUAGCUUCUUGCCCCUUGGGCCUCCCCCCAGCCCCUCC |
| | UCCCCUUCCUGCACCCGUACCCCCCAAACACCAUUGUCACACUCCAGUGGUCUUUGAAUAAAGU |
| | CUGAGUGGGCGGC |
| | (miR122 binding site boldened) |

(stop cassette is italicized; miR binding sites are boldened)

### EQUIVALENTS

All references, patents and patent applications cited herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A messenger ribonucleic acid (mRNA) vaccine comprising:
a first mRNA comprising a first open reading frame (ORF) encoding a first SARS-CoV-2 spike protein, wherein the SARS-CoV-2 spike protein comprises at least one of the following mutations relative to SEQ ID NO: 5: L24(del) or A27S.

2. The mRNA vaccine of claim 1, wherein the first SARS-CoV-2 spike protein comprises a L24(del) mutation relative to SEQ ID NO: 5.

3. The mRNA vaccine of claim 1, wherein the first SARS-CoV-2 spike protein comprises an A27S mutation relative to SEQ ID NO: 5.

4. The mRNA vaccine of claim 1, wherein the first SARS-CoV-2 spike protein comprises L24(del) and A27S mutations relative to SEQ ID NO: 5.

5. The mRNA vaccine of claim 1, wherein the first SARS-CoV-2 spike protein further comprises at least one of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K.

6. The mRNA vaccine of claim 5, wherein the first SARS-CoV-2 spike protein further comprises the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K.

7. The mRNA vaccine of claim 1, wherein the first SARS-CoV-2 spike protein comprises an amino acid sequence having at least 98% identity to SEQ ID NO: 12.

8. The mRNA vaccine of claim 1, wherein the first ORF comprises a nucleotide sequence that has at least 89% identity to the nucleotide sequence of SEQ ID NO: 10.

9. (canceled)

10. The mRNA vaccine of claim 1, further comprising a second mRNA comprising a second ORF encoding a second SARS-CoV-2 spike protein.

11. The mRNA vaccine of claim 10, wherein the second SARS-CoV-2 spike protein is different from the first SARS-CoV-2 spike protein.

12. The mRNA vaccine of claim 10, wherein the amino acid sequence of the second SARS-CoV-2 spike protein is at least 95% identical to the amino acid sequence of the first SARS-CoV-2 spike protein.

13. The mRNA vaccine of claim 10, wherein the first mRNA and the second mRNA are present in the mRNA vaccine at a ratio of 1:1.

14. The mRNA vaccine of claim 1, wherein the mRNA vaccine comprises 25 μg-75 μg of mRNA in total.

15. (canceled)

16. The mRNA vaccine of claim 1, wherein the first mRNA and, optionally, the second mRNA, comprises a chemical modification.

17. (canceled)

18. The mRNA vaccine of claim 16, wherein the chemical modification is 1-methylpseudouridine.

19. The mRNA vaccine of claim 1, further comprising a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid.

20.-25. (canceled)

26. A method comprising:
administering to a subject a mRNA vaccine comprising a first mRNA comprising a first open reading frame (ORF) encoding a first SARS-CoV-2 spike protein, wherein the SARS-CoV-2 spike protein comprises at least one of the following mutations relative to SEQ ID NO: 5: L24(del) or A27S.

27. The method of claim 26, wherein the first SARS-CoV-2 spike protein further comprises at least one of the following mutations relative to SEQ ID NO: 5: T19I, P25(del), P26(del), G142D, S371F, S373P, S375F, T376A, D405N, R408S, K417N, N440K, S477N, T478K, E484A, Q498R, N501Y, Y505H, D614G, H655Y, N679K, P681H, N764K, D796Y, Q954H, and N969K.

28. The method of claim 26, wherein the first mRNA is fully chemically modified.

29. The method of claim 26, wherein the mRNA vaccine further comprises a lipid nanoparticle, optionally wherein the lipid nanoparticle comprises 40-55 mol % ionizable amino lipid, 30-45 mol % sterol, 5-15 mol % neutral lipid, and 1-5 mol % PEG-modified lipid.

30. (canceled)
